SEQUENCE LISTING (6 sequences)

SEQ ID NO: 1 - protein, 127 aa, Mus musculus
MKTALLVLLVLAVATSPAWALRCHVCTNSANCKNPQVCPSNFYFCKTVTSVEPLNGNLVRKECANSCTSDYSQQGHVSSGSEVTQCCQTDLCNERLVSAAPGHALLSSVTLGLATSLSLLTVMALCL

SEQ ID NO: 2 - protein, 780 aa, Mus musculus
MDKFWWHTAWGLCLLQLSLAHPHQQIDLNVTCRYAGVFHVEKNGRYSISRTEAADLCQAFNSTLPTMDQMKLALSKGFETCRYGFIEGNVVIPRIHPNAICAANHTGVYILVTSNTSHYDTYCFNASAPPEEDCTSVTDLPNSFDGPVTITIVNRDGTRYSKKGEYRTHQEDIDASNIIDDDVSSGSTIEKSTPESYILHTYLPTEQPTGDQDDSFFIRSTLATIASTVHSKSHAAAQKQNNWIWSWFGNSQSTTQTQEPTTSATTALMTTPETPPKRQEAQNWFSWLFQPSESKSHLHTTTKMPGTESNTNPTGWEPNEENEDETDKYPSFSGSGIDDDEDFISSTIASTPRVSARTEDNQDWTQWKPNHSNPEVLLQTTTRMADIDRISTSAHGENWTPEPQPPFNNHEYQDEEETPHATSTTPNSTAEAAATQQETWFQNGWQGKNPPTPSEDSHVTEGTTASAHNNHPSQRITTQSQEDVSWTDFFDPISHPMGQGHQTESKDTDSSHSTTLQPTAAPNTHLVEDLNRTGPLSVTTPQSHSQNFSTLHGEPEEDENHPTTSILPSSTKSGAKDARRGGSLPTDTTTSVEGYTFQYPDTMENGTLFPVTPAKTEVFGETEVTLATDSNVNVDGSLPGDRDSSKDSRGSSRTVTHGSELAGHSSANQDSGVTTTSGPMRRPQIPEWLIILASLLALALILAVCIAVNSRRRCGQKKKLVINGGNGTVEDRKPSELNGEASKSQEMVHLVNKEPSETPDQCMTADETRNLQSVDMKIGV

SEQ ID NO: 3 - DNA, 19 bp, Artificial Sequence
  Synthetic
agagaaggtaggacattct

SEQ ID NO: 4 - DNA, 19 bp, Artificial Sequence
  Synthetic
agagaaggtaggacattct

SEQ ID NO: 5 - DNA, 19 bp, Artificial Sequence
  Synthetic
ggtataacatgctgaccaa

SEQ ID NO: 6 - DNA, 19 bp, Artificial Sequence
  Synthetic
gagaatagctaaggagaaa

(19) United States
(12) Patent Application Publication
(10) Pub. No.: US 2014/0086939 A1
(43) Pub. Date: Mar. 27, 2014

(54) COMPOSITIONS AND METHODS FOR DETECTING AND TREATING CANCER

(71) Applicants: Michael Karin, La Jolla, CA (US); Guobin He, San Diego, CA (US)

(72) Inventors: Michael Karin, La Jolla, CA (US); Guobin He, San Diego, CA (US)

(21) Appl. No.: 14/002,083

(22) PCT Filed: Feb. 28, 2012
(86) PCT No.: PCT/US12/26943

§ 371 Date: Nov. 12, 2013

Related U.S. Application Data
(60) Provisional application No. 61/447,248, filed on Feb. 28, 2011.

(51) Int. Cl. A61K 39/395; C12Q 1/68; G01N 33/50; A61K 31/7088

(52) U.S. Cl. CPC A61K 39/39558; A61K 31/7088; C12Q 1/6886; G01N 33/5011
USPC 424/174.1; 435/325; 435/354; 435/370; 506/9; 514/44 A; 435/6.12; 435/7.1

(57) ABSTRACT

The invention provides methods for detecting the presence of cancer initiating cells in a tissue, methods for identifying test agent for reducing cancer, and methods for reducing cancer in a subject. The invention's methods are applicable to any cancer, and in particular to liver cancer. The invention also provides the isolation and characterization of pre-malignant hepatocellular carcinoma initiating cells (HICs). The invention further provides methods for isolating hepatocellular carcinoma initiating cells (HICs), methods for using the isolated cells for screening anti-cancer drugs, methods for using HIC markers for the early diagnosis of hepatocellular carcinoma, and methods for the prevention and/or delay of hepatocellular carcinoma by using agents that selectively deplete the number and/or malignant properties of HICs.

### CROSS-REFERENCE TO RELATED APPLICATIONS

This application claims priority under 35 U.S.C. §119(e) to co-pending U.S. Provisional Application Ser. No. 61/447,248, filed on Feb. 28, 2011, herein incorporated by reference in its entirety.

### GOVERNMENT INTEREST

This invention was made, in part, with government support under grant numbers CA113602, CA118165, ES004151, ES006376, awarded by the National Cancer Institute of the National Institutes of Health. The government has certain rights in the invention.

### FIELD OF THE INVENTION

The invention provides the isolation and characterization of pre-malignant hepatocellular carcinoma initiating cells (HICs). The invention further provides methods for isolating hepatocellular carcinoma initiating cells (HICs), methods for using the isolated cells for screening anti-cancer drugs, methods for using HIC markers for the early diagnosis of hepatocellular carcinoma, and methods for the prevention and/or delay of hepatocellular carcinoma by using agents that selectively deplete the number and/or malignant properties of HICs.

### BACKGROUND

Hepatocellular carcinoma (HCC), the most common form of liver cancer, is the fifth most ubiquitous cancer worldwide and third leading cause of cancer deaths. Even in the US the 5-year survival rate is 8.9%, making HCC the second most lethal cancer after pancreatic cancer. This lethality stands in striking contrast to the slow growth and progression of HCC and is mainly due to resistance to all existing anticancer agents, including ionizing radiation, lack of biomarkers for early detection of surgically resectable tumors and associated liver disease that renders HCC patients intolerant of toxic chemotherapeutics. Therefore, there remains a need for compositions and methods for the early diagnosis, prevention and delay of hepatocellular carcinoma.

### SUMMARY OF THE INVENTION

The invention provides an isolated population of mammalian hepatocellular carcinoma initiating cells (HICs). The invention also provides a composition comprising the isolated population of mammalian hepatocellular carcinoma initiating cells (HICs) described herein.

The invention also provides a method for producing an isolated population of mammalian hepatocellular carcinoma initiating cells (HICs), comprising a) treating liver tissue from a mammalian subject with collagenase to produce a composition comprising a population of aggregated hepatocellular cells and a population of non-aggregated hepatocellular cells, and b) isolating the population of aggregated hepatocellular cells from the composition, thereby producing an isolated population of mammalian hepatocellular carcinoma initiating cells (HICs). In one embodiment, the mammalian subject is a mouse, such as a mouse selected from the group of a DEN-treated mouse, a mouse that lacks expression of TAK1, and a mouse that lacks expression of TAK1 and p38. In another embodiment, the mammalian subject is human.

The invention also provides an isolated population of mammalian hepatocellular carcinoma initiating cells (HICs) produced by the methods disclosed herein.

The invention additionally provides a method for identifying a HIC marker gene, comprising determining the level of expression of a gene in a) an isolated population of mammalian hepatocellular carcinoma initiating cells (HICs), and b) control non-cancerous cells, wherein an altered level of gene expression in the HICs compared to the control cells identifies the gene as a HIC marker gene. In one embodiment, the HIC marker gene encodes an HIC cell surface marker antigen. In a further embodiment, the control cells are selected from hepatic oval cells and hepatic normal cells.

Also provided herein is a method for detecting the presence of hepatocellular carcinoma initiating cells (HICs) in a sample, comprising a) introducing an isolated population of mammalian hepatocellular carcinoma initiating cells (HICs) into a mammalian host mammalian subject to produce a treated subject, and b) detecting hepatocellular cancer (HCC) in the treated subject, thereby detecting the presence of hepatocellular carcinoma initiating cells (HICs) in the sample. In one embodiment, the sample comprises liver tissue.

The invention also provides a method for detecting the presence of hepatocellular carcinoma initiating cells (HICs) in a sample, comprising detecting in the sample an HIC marker gene. In one embodiment, the detecting step comprises determining an altered level of expression of the HIC marker gene in the sample compared to the level of expression of the HIC marker gene in a control sample. In a particular embodiment, the control sample is selected from hepatic oval cell sample and hepatic normal cell sample. In a further embodiment, the HIC marker gene encodes an HIC cell surface marker antigen, and wherein the detecting step comprises determining an altered level of expression of the HIC cell surface marker antigen in the sample compared to the level of expression of the HIC cell surface marker antigen in a control sample. In another embodiment, the control sample is selected from hepatic oval cell sample and hepatic normal cell sample. In a preferred embodiment, the sample comprises liver tissue.

The invention also provides a method for identifying a test agent as reducing hepatocellular carcinoma initiating cells (HICs), comprising a) contacting i) an isolated population of mammalian hepatocellular carcinoma initiating cells (HICs), with the test agent, and b) detecting at least one of i) reduced number of the HICs, and reduced malignancy of the HICs, wherein the detecting identifies the test agent as reducing hepatocellular carcinoma initiating cells (HICs). In one embodiment, the test agent is selected from the group of anti-cancer cytotoxin, antibody that specifically binds to a HIC cell surface marker antigen, RNA interference sequence that specifically binds to mRNA that encodes a HIC marker protein, and antisense sequence that encodes a HIC marker protein. In a further embodiment, the anti-cancer cytotoxin comprises a nucleotide sequence encoding herpes simplex virus thymidine kinase (HSVtk). In an alternative embodiment, the antibody that specifically binds to a HIC cell surface marker antigen is selected from the group of antibody that specifically binds to CD44, and antibody that specifically binds to CD44v6. In a particular embodiment, the test agent is covalently linked to an antibody that specifically binds to a HIC cell surface marker antigen. In yet another embodiment, the test agent further comprises a liposome. In one preferred embodiment, the liposome further comprises an antibody that specifically binds to a HIC cell surface marker antigen. In a particular embodiment, the HIC cell surface marker antigen is encoded by the HIC marker gene that is identified by one or more of the methods described herein.

The invention also provides a method for reducing hepatocellular carcinoma initiating cells (HICs) in a mammalian subject comprising administering to a subject in need thereof a therapeutic amount of an agent that reduces hepatocellular carcinoma initiating cells (HICs). In one embodiment, the method further comprises detecting at least one of a) reduced number of the HICs, and b) reduced malignancy of the HICs. In a particular embodiment, the method further comprises detecting reduced hepatocellular carcinoma (HCC) in the subject.

The invention also provides a method for determining progression of hepatocellular carcinoma initiating cells (HICs) into hepatocellular carcinoma (HCC) cells, comprising a) administering diethyl nitrosamine (DEN) to a C57BL/6 mouse to produce a donor mouse, b) isolating a population of hepatocyte cells from the donor mouse, c) introducing the isolated hepatocyte cell population into the liver of a MUP-uPA transgenic mouse to produce a treated mouse host, and determining the presence of HCC in the liver of the treated mouse host, wherein detection of HCC determines progression of HICs in the isolated hepatocyte cell population into HCC cells. In one embodiment, the HCC cells in the liver of the treated mouse host express increased levels of albumin compared to control non-tumor cells. In another embodiment, the HCC cells in the liver of the treated mouse host express increased levels of α-fetoprotein compared to control non-tumor cells. In a particular embodiment, the treated mouse host is male. In a more preferred embodiment, the male treated mouse host comprises a higher number of HCC tumors than the number in a control female treated mouse host. In yet another embodiment, the male treated mouse host comprises a higher number of tumors per liver than the number in a control female treated mouse host. In a further embodiment, the C57BL/6 donor mouse is female.

The invention provides a method for detecting the presence of cancer initiating cells in a tissue, comprising a) isolating aggregated cells from a tissue, b) introducing the aggregated cells into a mammalian host animal to produce a treated tissue, wherein the introducing is under conditions for producing cancer in the tissue, and c) detecting cancer in the treated tissue, thereby identifying the presence of cancer initiating cells in the aggregated cells. In one embodiment, the tissue is normal tissue. In another embodiment, the method further comprises purifying the cancer initiating cells from the aggregated cells. In yet a further embodiment, the method further comprises detecting in the aggregated cells an increased expression of one or more protein selected from the group consisting of Ly6D protein and CD44 protein compared to a normal cell. In another embodiment, the tissue is liver tissue.

The invention further provides a method for detecting the presence of cancer initiating cells in a tissue, comprising detecting increased expression of one or more protein selected from the group consisting of Ly6D protein and CD44 protein compared to a normal cell. In one embodiment, the cancer initiating cells is a liver cancer initiating cell. In a further embodiment, the cancer initiating cells is from a mammalian subject, such as a human. In a further embodiment, the detecting increased expression comprises detecting increased levels of one or more of the Ly6D protein and the CD44 protein. In another embodiment, the detecting comprises contacting the cell with antibody that specifically binds to one or more of the proteins selected from the group consisting of Ly6D protein and CD44 protein. In yet another embodiment, the detecting increased expression comprises detecting increased levels of mRNA encoding one or more of the Ly6D protein and the CD44 protein.

The invention also provides a method for identifying a test agent as reducing cancer, comprising a) providing i) a target cell that expresses one or more proteins selected from the group consisting of Ly6D protein and CD44 protein, and ii) a test agent, b) contacting the test agent with the target cell to produce a contacted cell, and c) detecting reduced expression of the one or more proteins selected from the group consisting of Ly6D protein and CD44 protein compared to a control cell, thereby identifying the test agent as reducing cancer. In one embodiment, the test agent comprises an antibody that specifically binds to one or more proteins selected from the group consisting of Ly6D protein and CD44 protein. In another embodiment, the test agent comprises an RNA interference sequence that specifically binds to mRNA that encodes one or more proteins selected from the group consisting of Ly6D protein and CD44 protein. In a further embodiment, the test agent comprises one or more antisense sequences selected from the group consisting of Ly6D antisense sequence and CD44 antisense sequence.

The invention also provides a method for reducing cancer in a subject, comprising a) providing i) a mammalian subject, and ii) a composition comprising an agent that reduces one or both of 1) expression of a protein selected from the group consisting of Ly6D protein and CD44 protein, and 2) biological activity of a protein selected from the group consisting of Ly6D protein and CD44 protein, and b) administering a therapeutic amount of the composition to the subject. In one embodiment, the mammalian subject does not comprise cancer in a target tissue. In another embodiment, the mammalian subject comprises cancer in a target tissue.

### DESCRIPTION OF THE DRAWINGS

FIG. 1. Aggregated cells isolated from DEN-treated mouse livers are enriched for liver cancer initiating cells.

FIG. 2. Expression of Ly6D, a cell surface protein, is elevated in aggregated relative to single cells.

FIG. 3. Ly6D antibody specifically targets liver cancer in vivo.

FIG. 4. Expression of CD44, a cancer stem cell marker, is elevated in aggregated cells relative to single cells from DEN-treated mice.

FIG. 5. Identification and isolation of liver cancer initiating cells.

FIG. 6. (A) Amino acid sequence of mouse Ly6D (SEQ ID NO:1) (GenBank NM_{—}010742.1). (B) Amino acid sequence of mouse CD44 (SEQ ID NO:2) (GenBank No. NM_{—}009851.2).

FIG. 7. shows the roles of NF-κB signaling in hepatocarcinogenesis.

FIG. 8: HIC identification and isolation from livers of DEN treated mice. Male mice were DEN-treated at 2 weeks of age and after 3 or 5 months, their livers were excised and collagenase digested. a. Hepatocyte suspensions and numbers of aggregates from livers of DEN-treated or control mice. b. Aggregated and non-aggregated hepatocytes were separated, photographed (upper panels) and introduced into MUP-uPA mice whose livers and spleens were examined for HCC after 5 months.

FIG. 9: HIC can give rise to HCC in normal BL6 mice treated with retrorsine followed by CCl4 to induce compensatory proliferation. HIC isolated from livers of DEN treated mice were transplanted via the spleen to BL6 mice pretreated with retrorsine. After transplantation, mice were repetitively treated or not with CCl4 to induce compensatory proliferation. After 5 months, HCC nodules appeared in CCl4-treated mice.

FIG. 10: Gene expression patterns in HIC, HCC and normal hepatocytes. Expression of indicated mRNAs was quantified by Q-RT-PCR in: 1) normal hepatocytes, 2) non-aggregated and 3) aggregated hepatocytes from DEN-initiated liver, 4) HCC cells. Results are averages ±s.d. (n=3).

FIG. 11: Expression plot comparing undifferentiated oval cells with primary hepatocytes (Shin, S. 2011). In blue, genes that are significantly upregulated in HIC compared to normal hepatocytes are shown. The overlap between oval cell-specific and HIC-specific genes is highly significant (p<3.5E-34).

FIG. 12: HIC from livers of Tak1^{Δhep} mice also form aggregates. Livers of one month old Tak1^{F/F} and Tak1^{Δhep} mice were subjected to collagenase digestion. Collagenase-resistant aggregates were detected only in Tak1^{Δhep} livers. These cells gave rise to HCC in transplanted MUP-uPA mice while non-aggregated cells did not generate tumors.

FIG. 13: CD44+ aggregated hepatocytes are responsible for HCC formation. a. Hepatocyte aggregates were examined for CD44 expression by IF. b. Aggregated hepatocytes were separated into CD44+ and CD44− cells that were analyzed for their ability to generate HCC in MUP-uPA mice.

FIG. 14: Pre-malignant lesions precede DEN-induced HCC. Male and female mice were injected with PBS or DEN at 15 days of age. At indicated time points, BrdU was administrated and 2 hrs later, mice were sacrificed. Livers were collected and processed for (A) H&E staining and (B) BrdU antibody staining. Arrows indicate borders of FAH and the bargraphs on the right are the quantitation of A and B. * p<0.05.

FIG. 15: Immunochemical analysis of FAH and HIC-containing aggregates. Paraffin-embedded sections of livers from PBS- or DEN-injected male mice were stained with antibodies for the indicated antigens.

FIG. 16: A. The experimental design. B. Stereo image of a representative liver lobe from MUP-uPA mouse transplanted with HIC that were cultured briefly and infected with GFP lentivirus (left) and a non-transplanted control (right). Images were taken 5 weeks after HIC transplantation. Insets show GFP⁺ nodules.

FIG. 17: PCNA⁺, Sox9⁺ and EpCAM⁺ cells are present in cirrhotic nodules. Mag:400×.

FIG. 18. A transplant system for studying HCC progression. (A) A diagram of experimental protocol. C57BL/6 pups were given a single i.p. injection of DEN (25 mg/kg) when 15 days old. Hepatocytes were isolated 2-3 months later and transplanted into 3 weeks old MUP-uPA mice via intra-splenic injection. Recipients were sacrificed 5 months later for liver tumor analysis. (B) Representative livers of male MUP-uPA mice 5 months after transplantation with hepatocytes from vehicle- or DEN-treated male mice. Liver sections were stained with H&E and albumin antibody. Magnification bar=100 μm. (C) Relative expression of α-fetoprotein (AFP) mRNA was determined by real-time PCR in liver tumors (tumor) and surrounding non-tumor liver (Non-T). n=4; *: p<0.01 by t test. (D, E) DEN-treated male or (F, G) female mice were used as hepatocyte donors to MUP-uPA recipients of the indicated gender. Tumor incidence (D, F) and multiplicity (E, G) were determined at 5 months post-transplantation. n=8-10 for each group; *: p<0.01 by t test (E, G) or *: p<0.01 by chi-square test (D, F). Please also see FIG. 25.

FIG. 19. IKKβ deletion after initiation enhances HCC formation and growth. (A-C) Ikkβ^{f/f} male pups were DEN initiated and used as hepatocyte donors into MUP-uPA mice. IKKβ in transplanted hepatocytes was deleted 1 month later by injection of Adv-Cre. Adv-GFP was used as a control. Mice were sacrificed 4 months later and whole cell lysates were prepared from dissected HCCs and surrounding non-tumor livers (NT). (A) Lysates were gel separated and immunoblotted with the indicated antibodies or used for measurement of JNK kinase activity by immunecomplex kinase assay. Relative JNK kinase activity (KA) and ERK phosphorylation in the different samples were determined by densitometry and the average relative activities (RA) for each group of samples are indicated. (B) HCCs per liver were counted and (C) maximal tumor size was measured. n=7˜10 for each group; *: p<0.05. (D, E) Tumor cell proliferation and apoptosis were determined by PCNA (D) and TUNEL (E) staining, respectively, of paraffin embedded liver sections (n=10 each group; p<0.05). (F, G) Ikkβ^{f/f} (Cre⁻) and Ikkβ^{f/f}/Mx1-Cre (Cre⁺) DEN-initiated males were used as hepatocyte donors to male MUP-uPA recipients. IKKβ deletion was accomplished by poly(IC) injection 1 month after transplantation. HCC multiplicity (F) and maximal sizes (G) were determined 4 months later (n=10 each group; *: p<0.01). Please also see FIG. 26.

FIG. 20. IKKβ deletion in initiated cells enhances proliferation and self-renewal of HCC progenitors. (A) DEN-induced HCC (dih) cell strains from Ikkβ^{f/f} mice were cultured and infected with Adv-GFP or Adv-Cre. Whole cell lysates of infected dih10 cells were immunoblotted for IKKβ (bottom panel). The cells were further cultured without serum on Petri dishes to form hepatospheres (upper panels). Numbers of 1⁰ and 2⁰ hepatospheres formed per 100 plated cells were determined (n=3; *: p<0.05). Magnification bar=100 μm. (B) Ikkβ^{f/f} dih10 cells were infected with an empty retrovirus (vector) or a retrovirus expressing IκBα super-repressor (SR). Stably transfected cells were selected and cultured as above and hepatosphere formation was analyzed (n=3; *: p<0.05). (C, D) IKKβ expressing (Ikkβ^{f/f}) and deficient (Ikkβ^{Δ}) dih10 cells (2.5×10⁶ each) were s.c. injected into 8 weeks old C57BL/6 mice. (C) Allograft volume was measured weekly (n=5; *: p<0.01). Dissected tumors are shown on the right. (D) BrdU incorporation into tumors was determined (n=5; *: p<0.05). (E) Ikkβ^{f/f} and Ikkβ^{Δ} dih12 cells were labeled with dsRed and 4×10⁵ cells were seeded into MUP-uPA mouse livers. Relative amounts of dsRed DNA in transplanted MUP-uPA livers were determined by real-time PCR 3 weeks post-inoculation and normalized to actin DNA as a measurement of cell growth (n=5; *: p<0.05). (F) Ikkβ^{Δ} dih10 cells were reconstituted with a control vector or WT IKKβ expression vector and were s.c. injected into 8 weeks old C57BL/6 mice. Allograft volume was measured weekly (n=5; *: p<0.01). (G) Ikkβ^{f/f} and Ikkβ_{Δ} dih10 cells were s.c. injected as above. Starting from day 2, mice were treated daily with MLN120B or vehicle by oral gavage. Tumor volume was measured weekly (n=5; *: p<0.01, f/f/control vs. f/f/MLN by one-way ANOVA). Please also see FIG. 27.

FIG. 21. STAT3 is activated in the absence of IKKβ independently of JNK. (A) Tumors derived from Ikkβ^{f/f} (f/f) and Ikkβ^{Δ} (Δ) dih10 cells were collected and lysed. Tumor lysates were examined for JNK kinase activity (KA) and STAT3 phosphorylation. (B, C) Ikkβ^{f/f} and Ikkβ^{Δ} dih10 cells were infected with lentiviruses expressing either a control shRNA (control) or shRNAs against mouse Jnk1/2 (shJnk1/2) and implanted s.c. (B) Tumor growth was measured (n=5; p<0.05, Δ/control vs. Δ/shJnk1/2 by one-way ANOVA). (C) Tumors were collected and lysed. Lysates were examined for expression and phosphorylation of the indicated proteins. Please also see FIG. 28.

FIG. 22. ROS-mediated SHP1/2 inhibition in IKKβ-deficient HCCs correlates with STAT3 activation and accelerated tumor growth. (A) Tumors derived from Ikkβ^{f/f} (f/f) and Ikkβ^{Δ} (Δ) dih10 cells were collected and lysed. Tumor lysates were immunoprecipitated with an anti-JAK2 antibody and examined for JAK2 tyrosine phosphorylation with PY20 antibody. Tumor lysates were also used for immunoblot analyses with the indicated antibodies. (B) SHP1 and SHP2 were immunoprecipitated from above tumor lysates and their phosphatase activities were measured (n=4; *: p<0.05). (C) Fresh frozen sections of HCCs from transplanted MUP-uPA mice were analyzed for superoxide accumulation by DHE staining. Fluorescence intensity in several fields was quantitated by ImageJ and relative average increases in fluorescent intensity are shown (n=3; *: p<0.01). Magnification bar=100 μm. (D-F) Ikkβ^{f/5} (f/f) and Ikkβ^{Δ} (Δ) dih10 cells 2.5×10⁸ each were s.c. implanted into 8 weeks old C57BL/6 mice. Starting on day 2, the mice were switched to a diet containing vehicle or BHA (0.7%) for 6 weeks. (D) Tumors were collected from BHA treated and untreated mice, lysed and SHP1/2 phosphatase activities were measured. The data were plotted as SHP1/2 phosphatase activities in Ikkβ^{Δ} tumors relative to activities in Ikkβ^{f/f} tumors (n=4; *: p<0.05). (E) Tumor lysates were used to determine JNK kinase activity and STAT3 phosphorylation. (F) Tumor volume was measured weekly (n=5; p<0.01, Δ/ctrl vs. Δ/BHA by one way ANOVA). Please also see FIG. 29.

FIG. 23. STAT3 is required for HCC formation and growth. (A) Ikkβ^{f/f} (f/f) and Ikkβ^{Δ} (Δ) dih10 cells were infected with lentiviruses expressing either scrambled shRNA (ctrl) or an shRNA against mouse Stat3 (shStat3). Stably transfected cells were selected and 2.5×10⁶ cells were s.c. implanted. Tumor growth was measured (n=5; p<0.01, f/f/ctrl vs. f/f/shSTAT3; p<0.01, Δ/ctrl vs. Δ/shSTAT3 by one-way ANOVA). (B-D) Stat3^{f/f} and Stat3^{Δhep} male mice were injected with 25 mg/kg DEN when 15 days old. Mice were sacrificed 8 months later and HCC induction was evaluated. (B) HCCs and surrounding non-tumor tissues were collected, lysed and STAT3 expression and phosphorylation were examined. (C) Tumor multiplicity (n=10; *: p<0.01) and (D) maximal tumor sizes (n=10; *: p<0.05) were determined. (E) STAT3 and NF-κB activation in human HCC. Upper panels: representative samples of non-tumor liver tissue and liver tissue containing HCC were stained with a phospho-STAT3 antibody. Lower panels: adjacent parallel sections of the same samples shown in the upper panels were stained with a phospho-p65 antibody. The bar graphs present the frequency of phospho-STAT3 positive HCC specimens amongst all HCCs or amongst p65-positive and p65-negative HCCs (* p<0.05 by chi-square analysis). Please also see FIG. 30 and FIG. 31 (Table S1).

FIG. 24. A central role for IKKβ and ROS-controlled STAT3 signaling in HCC development. Inactivation of IKKβ or other anti-oxidant defenses in hepatocytes favors ROS accumulation and leads to oxidative inhibition of PTPs, including SHP1 and SHP2. This results in activation of JNK and STAT3 which stimulate the proliferation of initiated pre-neoplastic hepatocytes. This contributes both to early tumor promotion and HCC progression. In addition, STAT3 activation suppresses apoptosis in progressing HCCs.

FIG. 25. MUP-uPA transgenic mouse liver exhibits mild fibrosis, elevated IL-6 mRNA and is permissive to growth of transplanted hepatocytes. (A) Livers from 8-month-old WT and MUP-uPA transgenic mice were paraffin embedded, sectioned and stained with H&E and Sirius red (magnification: 100×). Magnification bar=100 μm. (B) Total RNA was extracted from livers of WT and MUP-uPA (1 month old) and the relative amount of IL-6 mRNA was determined by RT-PCR (n=4; *: p=0.011). (C) ROS accumulation in WT and MUP-uPA livers. Fresh frozen liver sections from 1 month old WT and MUP-uPA mice were stained with DHE to determine ROS accumulation. Images were quantified with ImageJ software (n=3; *: p=0.007). (D) Hepatocytes were isolated from DEN-treated 3 months old actin-GFP transgenic mice and transplanted into MUP-uPA mice via intra-splenic injection. Recipient livers were collected 1 month later and presence of transplanted GFP-expressing hepatocytes was examined by H&E staining and fluorescent microscopy (magnification: 200×). Magnification bar=100 μm.

FIG. 26. Adenovirus administration to hepatocyte-transplanted MUP-uPA mice results in mild liver injury. Hepatocytes from DEN-initiated Ikkβ^{f/f} males were transplanted into 3 weeks old MUP-uPA male transgenic mice. One month later, 1×10⁹ pfu of Adv-GFP or Adv-Cre were given to each transplanted mouse via the tail vein. Serum was collected before and 48 hrs after virus infection and ALT activity was measured. Results are averages ±s.d. (n=5).

FIG. 27. Culturing and characterization of HCC-derived hepatoma cells. (A) Three HCC-derived strains (dih 10-12) were analyzed by immunoblotting for expression of albumin and AFP. Normal primary hepatocytes were used as a control. (B) dih10 and dih12 cells at passage 3 were stained with albumin antibody and DAPI (magnification: 100×). Magnification bar=100 μm. (C) Normal primary hepatocytes (N) and HCC-derived dih10 cells (T) were cultured. Cell lysates were immunoblotted with indicated antibodies. (D) Ikkβ^{f/f} and Ikkβ^{Δ} dih10 cells were plated as single cell suspensions. Cell colonies were photographed 3 days later (magnification: 100×). Magnification bar=100 μm.

FIG. 28. IKKβ ablation enhances STAT3 activation. (A) Ikkβ^{f/f} and Ikkβ^{Δ} dih10 cells were cultured under hepatosphere-forming conditions for 3 days and treated with 10 ng/ml of IL-6 for the indicated times. Whole cell lysates were analyzed by immunoblotting for STAT3 phosphorylation. (B) Ikkβ^{f/f} and Ikkβ^{Δ} dih10 cells were cultured under hepatosphere-forming conditions for 3 days and treated with 10 ng/ml of IL-22 for 30 min. STAT3 phosphorylation was analyzed as above. (C) Ikkβ^{Δ} dih10 cells were cultured under hepatosphere-forming conditions and infected with a control Adv or an Adv vector expressing constitutively active IKKβ^{EE}. The cells were treated with 10 ng/ml of IL-6 for 15 min and STAT3 phosphorylation was analyzed. (D) Ikkβ^{f/f} dih10 cells and HepG2 cells were serum starved for 48 hrs and treated with 20 μM MLN120B for the indicated times. The cells were then stimulated with 10 ng/ml of IL-6 for 30 min and STAT3 phosphorylation was analyzed by immunoblotting. Relative activities (RA) of STAT3 phosphorylation were determined by densitometry. (E) Ikkβ^{f/f} (f/f) and Ikkβ^{Δ} (Δ) HCCs from transplanted MUP-uPA mice were collected and total RNA was extracted. Expression of IL-6 and IL-22 mRNAs was measured by RT PCR.

FIG. 29. Elevated STAT3 activity in IKKβ-deficient HCCs is due to enhanced JAK2 activation and reduced SHP1/2 activity and correlates with ROS accumulation. (A) Ikkβ^{f/f} (f/f) and Ikkβ^{Δ} (Δ) dih10 cells were infected with lentiviruses expressing either a scrambled shRNA (−) or an shRNA against mouse Jak2 (+). After 48 hrs, the cells were treated with vehicle or 10 ng/ml of IL-6 for 30 min. The cells were lysed and lysates were analyzed by immunoblotting with the indicated antibodies. (B) Ikkβ^{f/f}(f/f) and Ikkβ^{Δ} (Δ) HCCs from transplanted MUP-uPA mice were collected and total RNA was extracted. Expression of SOCS3 mRNAs was measured by RT PCR (n=4; *: p<0.05). (C) HCCs derived from vector or Ikkβ-reconstituted Ikkβ^{Δ} dih10 cells (from FIG. 20F) were lysed. SHP1 and SHP2 were immunoprecipitated and their phosphatase activities were measured (n=4; *: p<0.05). (D) Ikkβ^{f/f} (f/f) and Ikkβ^{Δ} (Δ) dih10 cells were infected with adenovirus expressing either GFP or SHP2. After 24 hrs, the cells were treated with vehicle or 10 ng/ml of IL-6 for 30 min. The cells were lysed and subjected to immunoblot analysis with the indicated antibodies. (E) The conserved catalytic motif of SHP1 and SHP2 compared to that of other PTPases. The position of the catalytic cysteine, which is highly susceptible to oxidation, is indicated. (F, G) Ikkβ^{f/f} and Ikkβ^{Δ} dih10 cells were cultured without serum (but with EGF). (F) ROS accumulation in response to IL-6 (20 ng/ml) or additional EGF (20 ng/ml) was measured by DCFDA staining and fluorometry at 4 hrs (n=3; *: p<0.05 compared to vehicle control). (G) Ikkβ^{Δ} dih10 cells were infected with Adv-IKKβ^{EE} or an empty Adv vector. After 24 hrs, ROS accumulation in response to IL-6 and EGF was measured as above (n=3; *: p<0.05 compared to vector-infected control).

FIG. 30. STAT3 is required for HCC formation and growth. (A-D) Ikkβ^{f/f} (f/f) and Ikkβ^{Δ} (Δ) dih10 cells were s.c. implanted into 8 weeks old C57BL/6 mice. (A) Starting on day 2, the mice were i.p. injected with vehicle (ctrl) or 0.5 mg/mouse AG490 daily. Tumor volume was measured (n=5; p<0.05, f/f/vehicle vs. f/f/AG490; p<0.01, Δ/vehicle vs. Δ/AG490 by one way ANOVA). (B) After 6 weeks, tumors were removed, lysed and analyzed by immunoblotting with the indicated antibodies. (C) Starting on day 2, the mice were i.p. injected with vehicle (ctrl) or 5 mg/kg S3I-201 daily. Tumor volume was measured (n=5; p<0.05, f/f/vehicle vs. f/f/S3I-201; p<0.01, Δ/vehicle vs. Δ/S3I-201 by one way ANOVA). (D) After 6 weeks, tumors were removed and analyzed as above. (E) Lysates of Ikkβ^{f/f} (f/f) and Ikkβ^{Δ} (Δ) dih10 cells that were infected with either control or Stat3 shRNA lentiviruses were analyzed for STAT3 expression by immunoblotting. (F, G) DEN-induced HCCs from Stat3^{f/f} mice were cultured as described in Methods. Cells were infected with Adv-GFP or Adv-Cre overnight. (F) STAT3 protein expression was examined by immunoblotting 5 days after infection and (G) the cells were photographed 10 days after infection. Magnification bar=100 μm.

FIG. 31. Table shows correlation between clinical parameters and hoph-STAT staining in human HCC.

### DEFINITIONS

To facilitate understanding of the invention, a number of terms are defined below.

As used in this specification and the appended claims, the singular forms “a,” “an” and “the” includes both singular and plural references unless the content clearly dictates otherwise.

As used herein, the term “or” when used in the expression “A or B,” where A and B refer to a composition, disease, product, etc., means one, or the other, or both.

The term “on” when in reference to the location of a first article with respect to a second article means that the first article is on top and/or into the second article, including, for example, where the first article permeates into the second article after initially being placed on it.

As used herein, the term “comprising” when placed before the recitation of steps in a method means that the method encompasses one or more steps that are additional to those expressly recited, and that the additional one or more steps may be performed before, between, and/or after the recited steps. For example, a method comprising steps a, b, and c encompasses a method of steps a, b, x, and c, a method of steps a, b, c, and x, as well as a method of steps x, a, b, and c. Furthermore, the term “comprising” when placed before the recitation of steps in a method does not (although it may) require sequential performance of the listed steps, unless the content clearly dictates otherwise. For example, a method comprising steps a, b, and c encompasses, for example, a method of performing steps in the order of steps a, c, and b, the order of steps c, b, and a, and the order of steps c, a, and b, etc.

Unless otherwise indicated, all numbers expressing quantities of ingredients, properties such as molecular weight, reaction conditions, and so forth as used in the specification and claims are to be understood as being modified in all instances by the term “about.” Accordingly, unless indicated to the contrary, the numerical parameters in the specification and claims are approximations that may vary depending upon the desired properties sought to be obtained by the present invention. At the very least, and without limiting the application of the doctrine of equivalents to the scope of the claims, each numerical parameter should at least be construed in light of the number of reported significant digits and by applying ordinary rounding techniques. Notwithstanding that the numerical ranges and parameters describing the broad scope of the invention are approximation, the numerical values in the specific examples are reported as precisely as possible. Any numerical value, however, inherently contains standard deviations that necessarily result from the errors found in the numerical value's testing measurements.

The term “not” when preceding, and made in reference to, any particularly named molecule (such as a protein, nucleotide sequence, etc.) or phenomenon (such as cell adhesion, cell migration, cell differentiation, angiogenesis, biological activity, biochemical activity, etc.) means that only the particularly named molecule or phenomenon is excluded.

A “cancer cell” refers to a cell undergoing early, intermediate or advanced stages of multi-step neoplastic progression as previously described (H. C. Pitot (1978) in “Fundamentals of Oncology,” Marcel Dekker (Ed.), New York pp 15-28). The features of early, intermediate and advanced stages of neoplastic progression have been described using microscopy. Cancer cells at each of the three stages of neoplastic progression generally have abnormal karyotypes, including translocations, inversion, deletions, isochromosomes, monosomies, and extra chromosomes. A cell in the early stages of malignant progression is referred to as “hyperplastic cell” and is characterized by dividing without control and/or at a greater rate than a normal cell of the same cell type in the same tissue. Proliferation may be slow or rapid but continues unabated. A cell in the intermediate stages of neoplastic progression is referred to as a “dysplastic cell.” A dysplastic cell resembles an immature epithelial cell, is generally spatially disorganized within the tissue and loses its specialized structures and functions. During the intermediate stages of neoplastic progression, an increasing percentage of the epithelium becomes composed of dysplastic cells. “Hyperplastic” and “dysplastic” cells are referred to as “pre-neoplastic” cells. In the advanced stages of neoplastic progression a dysplastic cell become a “neoplastic” cell. Neoplastic cells are typically invasive i.e., they either invade adjacent tissues, or are shed from the primary site and circulate through the blood and lymph to other locations in the body where they initiate one or more secondary cancers, i.e., “metastases.” Thus, the term “cancer” is used herein to refer to a malignant neoplasm, which may or may not be metastatic.

Malignant neoplasms within the scope of the invention include, for example, carcinomas such as liver cancer, lung cancer, breast cancer, prostate cancer, cervical cancer, pancreatic cancer, colon cancer, ovarian cancer; stomach cancer, esophagus cancer, mouth cancer, tongue cancer, gum cancer, skin cancer (e.g., melanoma, basal cell carcinoma, Kaposi's sarcoma, etc.), muscle cancer, heart cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, lymphoma, and leukemia. Malignant neoplasms are further exemplified by sarcomas (such as osteosarcoma and Kaposi's sarcoma).

The terms “reduce,” “inhibit,” “diminish,” “suppress,” “decrease,” and grammatical equivalents (including “lower,” “smaller,” etc.) when in reference to the level of any molecule (e.g., amino acid sequence such as α-fetoprotein, albumin, Ly6D protein and CD44 protein, anti-Ly6D antibody, anti-CD44 antibody, etc., and nucleic acid sequence such as those encoding any of the polypeptides described herein), cell (e.g., cancer cell, normal cell, metastatic cell, cancer initiating cells, stem cells, etc.) and/or phenomenon (e.g., malignancy, binding to a molecule, affinity of binding, expression of a nucleic acid sequence, transcription of a nucleic acid sequence, enzyme activity, etc.) in a first sample (or patient) relative to a second sample (or in a treated patient), mean that the quantity of molecule, cell and/or phenomenon in the first sample is lower than in the second sample by any amount that is statistically significant using any art-accepted statistical method of analysis. In one embodiment, the quantity of molecule, cell and/or phenomenon in the first sample is at least 10% lower than, at least 25% lower than, at least 50% lower than, at least 75% lower than, and/or at least 90% lower than the quantity of the same molecule, cell and/or phenomenon in a second sample. In another embodiment, the quantity of molecule, cell, and/or phenomenon in the first sample is lower by any numerical percentage from 5% to 100%, such as, but not limited to, from 10% to 100%, from 20% to 100%, from 30% to 100%, from 40% to 100%, from 50% to 100%, from 60% to 100%, from 70% to 100%, from 80% to 100%, and from 90% to 100% lower than the quantity of the same molecule, cell and/or phenomenon in a second sample.

The terms “increase,” “elevate,” “raise,” and grammatical equivalents (including “higher,” “greater,” etc.) when in reference to the level of any molecule (e.g., amino acid sequence such as α-fetoprotein, albumin, Ly6D protein and CD44 protein, anti-Ly6D antibody, anti-CD44 antibody, etc., and nucleic acid sequence such as those encoding any of the polypeptides described herein), cell (e.g., cancer cell, normal cell, metastatic cell, cancer initiating cells, stem cells, etc.) and/or phenomenon (e.g., malignancy, binding to a molecule, affinity of binding, expression of a nucleic acid sequence, transcription of a nucleic acid sequence, enzyme activity, etc.) in a first sample (or patient) relative to a second sample (or in a treated patient), mean that the quantity of the molecule, cell and/or phenomenon in the first sample is higher than in the second sample by any amount that is statistically significant using any art-accepted statistical method of analysis. In one embodiment, the quantity of the molecule, cell and/or phenomenon in the first sample is at least 10% greater than, at least 25% greater than, at least 50% greater than, at least 75% greater than, and/or at least 90% greater than the quantity of the same molecule, cell and/or phenomenon in a second sample. This includes, without limitation, a quantity of molecule, cell, and/or phenomenon in the first sample that is at least 10% greater than, at least 15% greater than, at least 20% greater than, at least 25% greater than, at least 30% greater than, at least 35% greater than, at least 40% greater than, at least 45% greater than, at least 50% greater than, at least 55% greater than, at least 60% greater than, at least 65% greater than, at least 70% greater than, at least 75% greater than, at least 80% greater than, at least 85% greater than, at least 90% greater than, and/or at least 95% greater than the quantity of the same molecule, cell and/or phenomenon in a second sample.

The term “substantially the same” mean that the difference in quantity of measurement or phenomenon in the first sample compared to the second sample is not statistically significant. In one embodiment, the difference in quantity of measurement or phenomenon between the first and second samples is less than 10%.

Reference herein to any numerical range expressly includes each numerical value (including fractional numbers and whole numbers) encompassed by that range. To illustrate, and without limitation, reference herein to a range of “at least 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc. In yet another illustration, reference herein to a range of from “5 to 10” includes each whole number of 5, 6, 7, 8, 9, and 10, and each fractional number such as 5.1, 5.2, 5.3, 5.4, 5.5, 5.6, 5.7, 5.8, 5.9, etc.

Reference herein to any specifically named protein (such as Ly6D protein, CD44 protein, etc.) refers to a polypeptide having at least one of the biological activities (such as those disclosed herein and/or known in the art) of the specifically named protein, wherein the biological activity is detectably by any method. In a preferred embodiment, the amino acid sequence of the polypeptide has at least 95% homology (i.e., identity) with the amino acid sequence of the specifically named protein. Reference herein to any specifically named protein (such as Ly6D protein, CD44 protein, etc.) also includes within its scope fragments, fusion proteins, and variants of the specifically named protein that have at least 95% homology with the amino acid sequence of the specifically named protein.

The term “fragment” when in reference to a protein refers to a portion of that protein that may range in size from four (4) contiguous amino acid residues to the entire amino acid sequence minus one amino acid residue. Thus, a polypeptide sequence comprising “at least a portion of an amino acid sequence” comprises from four (4) contiguous amino acid residues of the amino acid sequence to the entire amino acid sequence.

The term “variant” of a protein as used herein is defined as an amino acid sequence which differs by insertion, deletion, and/or conservative substitution of one or more amino acids from the protein. The term “conservative substitution” of an amino acid refers to the replacement of that amino acid with another amino acid which has a similar hydrophobicity, polarity, and/or structure. For example, the following aliphatic amino acids with neutral side chains may be conservatively substituted one for the other: glycine, alanine, valine, leucine, isoleucine, serine, and threonine. Aromatic amino acids with neutral side chains which may be conservatively substituted one for the other include phenylalanine, tyrosine, and tryptophan. Cysteine and methionine are sulphur-containing amino acids which may be conservatively substituted one for the other. Also, asparagine may be conservatively substituted for glutamine, and vice versa, since both amino acids are amides of dicarboxylic amino acids. In addition, aspartic acid (aspartate) my be conservatively substituted for glutamic acid (glutamate) as both are acidic, charged (hydrophilic) amino acids. Also, lysine, arginine, and histidine my be conservatively substituted one for the other since each is a basic, charged (hydrophilic) amino acid. Guidance in determining which and how many amino acid residues may be substituted, inserted or deleted without abolishing biological and/or immunological activity may be found using computer programs well known in the art, for example, DNAStar™ software. In one embodiment, the sequence of the variant has at least 95% identity, preferably at least 90% identity, more preferably at least 85% identity, yet more preferably at least 75% identity, even more preferably at least 70% identity, and also more preferably at least 65% identity with the sequence of the protein in issue.

Reference herein to any specifically named nucleotide sequence (such as a sequence encoding Ly6D protein, encoding CD44 protein, etc.) includes within its scope fragments, homologs, and sequences that hybridize under high and/or medium stringent conditions to the specifically named nucleotide sequence, and that have at least one of the biological activities (such as those disclosed herein and/or known in the art) of the specifically named nucleotide sequence, wherein the biological activity is detectable by any method.

The nucleotide “fragment” may range in size from an exemplary 10, 20, 50, 100 contiguous nucleotide residues to the entire nucleic acid sequence minus one nucleic acid residue. Thus, a nucleic acid sequence comprising “at least a portion of” a nucleotide sequence comprises from ten (10) contiguous nucleotide residues of the nucleotide sequence to the entire nucleotide sequence.

The term “homolog” of sequence of interest (e.g., nucleotide sequence, and/or amino acid sequence) refers to a sequence that has at least 90% identity with the sequence of interest, including at least 91% identity, at least 92% identity, at least 93% identity, at least 94% identity, at least 95% identity, at least 96% identity, at least 97% identity and at least 98% identity, with the sequence of interest. Homologs of nucleotide sequences include “orthologs,” i.e., genes in different species that evolved from a common ancestral gene by speciation. In some embodiments, orthologs retain the same function.

The terms “disease” and “pathological condition” are used interchangeably to refer to a state, signs, and/or symptoms that are associated with any impairment, interruption, cessation, or disorder of the normal state of a living animal or of any of its organs or tissues that interrupts or modifies the performance of normal functions, and may be a response to environmental factors (such as malnutrition, industrial hazards, or climate), to specific infective agents (such as worms, bacteria, or viruses), to inherent defect of the organism (such as various genetic anomalies, or to combinations of these and other factors. The term “disease” includes responses to injuries, especially if such responses are excessive, produce symptoms that excessively interfere with normal activities of an individual, and/or the tissue does not heal normally (where excessive is characterized as the degree of interference, or the length of the interference).

As used herein the terms “therapeutically effective amount,” “pharmaceutically effective amount,” and “protective amount” refer to an amount that delays, reduces, palliates, ameliorates, stabilizes, prevents and/or reverses one or more symptoms of a disease, such as inflammation, compared to in the absence of the composition of interest.

Specific “dosages” As used herein, the actual amount, i.e., “dosage,” encompassed by the terms “therapeutically effective amount,” “pharmaceutically effective amount,” and “protective amount” can be readily determined using animal models and in clinical trials and depend, for example, on the route of administration, subject weight (e.g. milligrams of drug per kg body weight), subject type (e., mammalian subject, non-mammalian subject, primate, non-primate, etc.), and the physical characteristics of the specific subject under consideration. These factors and their relationship to determining this amount are well known to skilled practitioners in the medical, veterinary, and other related arts. This amount and the method of administration can be tailored to achieve optimal efficacy but will depend on such factors as weight, diet, concurrent medication and other factors which those skilled in the art will recognize. The dosage amount and frequency are selected to create an effective level of the compound without substantially harmful effects.

“Subject” “and “animal” interchangeably refer to any multicellular animal, preferably a “mammal,” e.g., humans, non-human primates, murines, ovines, bovines, ruminants, lagomorphs, porcines, caprins, equines, canines, felines, ayes, etc.). Thus, mammalian subjects include mouse, rat, guinea pig, hamster, ferret and chinchilla. “Subject” also includes non-mammals, such as avians (e.g., chicken), amphibians (e.g. Xenopus), reptiles, and insects (e.g. Drosophila).

“Subject in need of” reducing one or more symptoms of a disease, e.g., cancer, etc., includes a subject that exhibits and/or is at risk of exhibiting one or more symptoms of the disease. For Example, subjects may be at risk based on family history, genetic factors, environmental factors, etc. This term includes animal models of the disease.

The terms “purified,” “isolated,” and grammatical equivalents thereof as used herein, refer to the reduction in the amount of at least one undesirable component (such as cell type, protein, and/or nucleic acid sequence) from a sample, including a reduction by any numerical percentage of from 5% to 100%, such as, but not limited to, from 10% to 100%, from 20% to 100%, from 30% to 100%, from 40% to 100%, from 50% to 100%, from 60% to 100%, from 70% to 100%, from 80% to 100%, and from 90% to 100%. Thus purification results in an “enrichment,” i.e., an increase in the amount of a desirable cell type, protein and/or nucleic acid sequence in the sample. Thus, an isolated component (e.g., isolated aggregated hepatocellular cells) does not necessarily mean, though it may include, a 100% reduction in the amount of one or more undesirable component (e.g., non-aggregated hepatocellular cells).

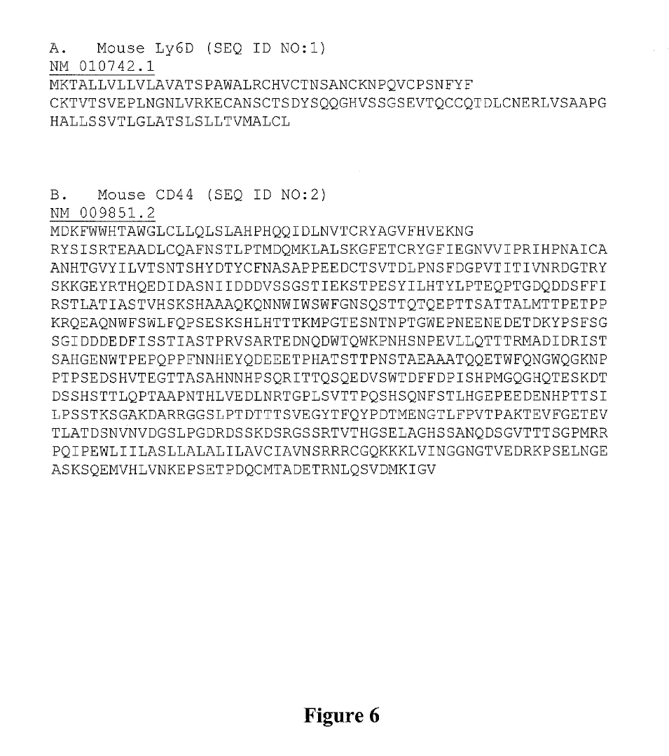

“Ly6D” refers to a glycosylphosphatidylinositol-anchored (GPI-anchored) cell membrane protein (10), and is exemplified by mouse Ly6D (SEQ ID NO:1) (GenBank NM_{—}010742.1) of FIG. 6, and its homologs.

“CD44” is a cell marker for cancer stem cells in some solid cancers (11), and is exemplified by mouse CD44 (SEQ ID NO:2) (GenBank No. NM_{—}009851.2) of FIG. 6, and its homologs. CD44 is encoded by a gene subjected to alternative splicing of at least 10 variant exons. The CD44v6 is an isoform of CD44 and is expressed in most human HCCs with poor clinical characteristics, but not in normal, and known to interact with c-Met, the HGF receptor.

“Aggregated cells” refers to a population of 2 or more cells that are in physical contact with each other, and that may be isolated from a tissue by dispersion of cells from the tissue, followed by filtering of the dispersed cells through a series of cell strainers, thereby enriching for aggregated cells and reducing the population of single cells.

The terms “cells” and “population of cells” interchangeably refer to a plurality of cells, i.e., more than one cell. The population may be a pure population comprising one cell type. Alternatively, the population may comprise more than one cell type. In the present invention, there is no limit on the number of cell types that a cell population may comprise

“HCC” refers to hepatocellular carcinoma.

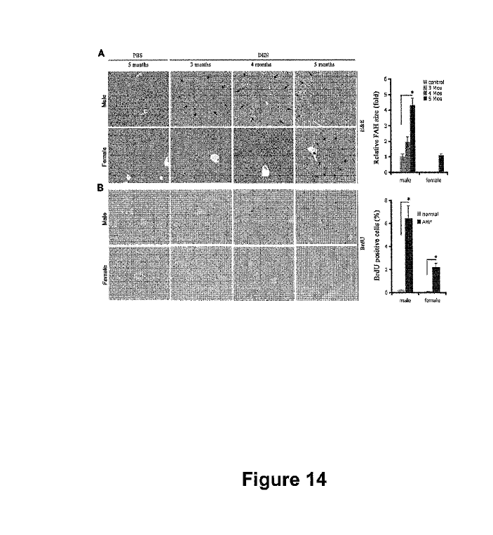

“HIC,” “HCC initiating cell,” “hepatocellular carcinoma initiating cell” and “hepatocyte initiated cells” are interchangeably used to refer to aggregated cells that express CD44 and that are able to progress into hepatocellular carcinoma (HCC). In one embodiment, HICs express CD44v6. HCC initiating cells may be isolated and characterized using methods disclosed herein in Examples 2-10. HCC initiating cells may be detected by their ability to form collagenase-resistant aggregates that give rise to HCC after transplantation into MUP-uPA or retrorsine+CCl₄-treated mice. HCC initiating cells may not be identical to HCC stem cells. First, HIC are not isolated from fully malignant tumor nodules. Second, HIC do not give rise to cancer when transplanted subcutaneously or even intrasplenically into normal BL6 mice. By contrast, HCC stem cells from established tumors give rise to subcutaneous, splenic or liver tumors when transplanted into normal BL6 mice²⁴. HIC only give rise to liver tumors when transplanted into either MUP-uPA mice or normal BL6 mice treated with retrorsine and CCl₄ and do not grow in any site other than the liver. Third, HIC do not form HCC immediately and usually require a 4-5-month latency period before HCC growth can be detected. Mouse HCC initiating cells differ from fully malignant hepatocellular carcinoma cells in that, in contrast to fully malignant mouse HCC cells, mouse HIC do not give rise to hepatocellular carcinoma when injected intrasplenically or subcutaneously into normal BL6 mice. In one embodiment, without limiting the invention to any particular mechanism, HIC are derived from “morphologically altered hepatocytes” (“FAH”), whose cells are more proliferative than the surrounding liver parenchyma and are smaller and more tightly packed than normal zone 3 hepatocytes (FIG. 14).

“Malignancy” of cells, such as of hepatocellular carcinoma initiating cells (HICs) refers to the ability of the cells to progress to carcinoma. For example, mouse HICs transferred into MUP-uPA mice, and/or into hybrid mice that are a cross of Tak1^{Δhep} and p38α^{Δhep} mice will progress to hepatocellular carcinoma.

“Non-aggregated cells” refers to a population of single cells.

“DEN” refers to diethyl nitrosamine.

A subject “at risk” for disease refers to a subject that is predisposed to contracting and/or expressing one or more symptoms of the disease. This predisposition may be genetic (e.g., a particular genetic tendency to expressing one or more symptoms of the disease, such as heritable disorders, etc.), or due to other factors (e.g., environmental conditions, exposures to detrimental compounds, including carcinogens, present in the environment, etc.). The term subject “at risk” includes subjects “suffering from disease,” i.e., a subject that is experiencing one or more symptoms of the disease. It is not intended that the present invention be limited to any particular signs or symptoms. Thus, it is intended that the present invention encompass subjects that are experiencing any range of disease, from sub-clinical symptoms to full-blown disease, wherein the subject exhibits at least one of the indicia (e.g., signs and symptoms) associated with the disease.

Cell “marker” molecule refer to a molecule (nucleotide sequence, protein sequence, antigen, etc.) that is present on, and/or is produced by, a particular type of cell (such as cancer cell, epithelial cell, fibroblast cell, muscle cell, synovial cell, stem cell, embryonic cell, etc.), at a different level (e.g., a higher level or lower lever, preferably a higher level) than other types of cells. Cell marker molecules may be used to distinguish one type of cell from other cell types. For example, “HIC marker antigen” refers to an antigen that is present on, and/or is produced by HICs at a different level (e.g., a higher level or lower lever, preferably a higher level) than other types of cells, such as normal cells and/or oval cells and/or hepatocellular carcinoma cells. “HIC cell surface marker antigen” refers to a HIC marker antigen that is expressed on the cell surface of a HIC. “HIC marker protein” refers to a protein that is present on, and/or is produced by HICs at a different level (e.g., a higher level or lower lever, preferably a higher level) than other types of cells, such as normal cells and/or oval cells and/or hepatocellular carcinoma cells.

“Sample” and “specimen” as used herein are used in their broadest sense to include any composition, such as a chemical reaction mixture, a composition from a biological and/or environmental source, as well as sampling devices (e.g., swabs) that have come into contact with these compositions. “Biological samples” include those obtained from a subject, including body fluids (such as urine, blood, plasma, fecal matter, cerebrospinal fluid (CSF), semen, sputum, and saliva), as well as solid tissue. Biological samples also include a cell (such as cell lines, cells isolated from tissue whether or not the isolated cells are cultured after isolation from tissue, fixed cells such as cells fixed for histological and/or immunohistochemical analysis), tissue (such as biopsy material), cell extract, tissue extract, and nucleic acid (e.g., DNA and RNA) isolated from a cell and/or tissue, and the like. These examples are illustrative, and are not to be construed as limiting the sample types applicable to the present invention.

A “control sample” refers to a sample used for comparing to another sample by maintaining the same conditions in the control and other samples, except in one or more particular variable in order to infer a causal significance of this varied one or more variable on a phenomenon. For example, a “positive control sample” is a control sample in which the phenomenon is expected to occur. For example, a “negative control sample” is a control sample in which the phenomenon is not expected to occur.

“Non-cancerous” cell and “non-malignant” cell interchangeably refer to a cell that does not progress into a cancer cell. Non-cancerous cell is exemplified by a normal cell and hepatic oval cell.

“Oval cell” refers to a liver cell that originates from a peri-portal location in response to injuries induced by BDL, CDE, diet supplemented with 3-diethoxycarbonyl-1,4-dihydrocollidine (DDC) and 2-acetylaminofluorene (2-AAF) (Thong et al. Science 1994; 264:95-8). Neither DEN nor CCl₄ are known to induce oval cells. Oval cells are not malignant. Procedures for oval cell isolation were previously described (Dorrell et al., Genes Dev 25, 1193-1203 (2011).

The term “liposome” as used herein refers to a lipid-containing vesicle having a lipid bilayer as well as other lipid carrier particles which can entrap antisense oligonucleotides. Liposomes may be made of one or more phospholipids, optionally including other materials such as sterols. Suitable phospholipids include phosphatidyl cholines, phosphatidyl serines, and many others that are well known in the art. Liposomes can be unilamellar, multilamellar or have an undefined lamellar structure.

“Cytotoxic” molecule refers any molecule that reduces proliferation and/or viability of a target cell, preferably, though not necessarily, killing the target cell. In a preferred embodiment, the cytotoxic molecule is an anti-cancer toxin.

“Anti-cancer toxin” and “anti-cancer cytotoxin” is a molecule that reduce proliferation and/or viability of cancer cells. In preferred embodiments, anti-cancer toxins delay the onset of development of tumor development and/or reduce the number, weight, volume, and/or growth rate of tumors. Cytotoxins are exemplified by, without limitation, second messengers such as cAMP; Bacterial toxins such as the exemplary Pertussis toxin, Cholera toxin, and C3 exoenzyme; Lectins such as Ricin A (Engert et al. Blood. 1997 Jan. 15; 89(2):403-10.). Also included are toxins exemplified by Topoisomerase inhibitors such as etoposide, Campothecin irinotecan, topotecan, anthracyclines (doxorubicine, daunorubicine); Microtubule inhibitors such as vincristine, vinblastine, vinorelbine, paclitaxel, docetaxel; Platinum containing compounds such as cisplatin, carboplatin, oxaloplatin, etc.; Alkylating agents such as cyclophosphamide, and ifosfamide; Antimetabolites such as methotrexate and mercaptopurine; Anti-estrogens such as tamoxifen and toremifene; Retinoids such as all trans-retinoic acid; and others such as Adriamycin, gemcitabine, and 5-fluoruracil.

A number of the above-mentioned toxins also have a wide variety of analogues and derivatives, including, but not limited to, cisplatin, cyclophosphamide, misonidazole, tiripazamine, nitrosourea, mercaptopurine, methotrexate, flurouracil, epirubicin, doxorubicin, vindesine and etoposide. Analogues and derivatives include (CPA).sub.2Pt(DOLYM) and (DACH)Pt(DOLYM) cisplatin, Cis-(PtCl.sub.2(4,7-H-5-methyl-7-oxo-)1,2,4(triazolo(1,5-a)pyrimidine).sub.2), (Pt(cis-1,4-DACH)(trans-Cl.sub.2)(CBDCA)).multidot.-1/2MeOH cisplatin, 4-pyridoxate diammine hydroxy platinum, Pt(II).Pt(II) (Pt.sub.2(NHCHN(C(CH.sub.2)(CH.s-ub.3))).sub.4), 254-S cisplatin analogue, O-phenylenediamine ligand bearing cisplatin analogues, trans, cis-(Pt(OAc).sub.21.sub.2(en)), estrogenic 1,2-diarylethylenediamine ligand (with sulfur-containing amino acids and glutathione) bearing cisplatin analogues, cis-1,4-diaminocyclohexane cisplatin analogues, 5′ orientational isomer of cis-(Pt(NH.sub.3)(4-aminoTEMP-O){d(GpG)}), chelating diamine-bearing cisplatin analogues, 1,2-diarylethyleneamine ligand-bearing cisplatin analogues, (ethylenediamine)platinum-(II) complexes, CI-973 cisplatin analogue, cis-diamminedichloroplatinum(II) and its analogues cis-1,1-cyclobutanedicarbosylato(2R)-2-methyl-1,4-butanediam-mineplatinum-(II) and cis-diammnine(glycolato)platinum, cis-amine-cyclohexylamine-dichloroplatinum(II), gem-diphosphonate cisplatin analogues (FR 2683529), (meso-1,2-bis(2,6-dichloro-4-hydroxyplenyl)ethylenediamine) dichloroplatinum(II), cisplatin analogues containing a tethered dansyl group, platinum(II) polyamines, cis-(3H)dichloro(ethylenediamine)platinum(II), trans-diamminedichloroplatinum(II) and cis-(Pt(NH.sub.3).sub.2(N.sub.3-cytosine)Cl), 3H-cis-1,2-diaminocyclohexanedichloroplatinum(II) and 3H-cis-1,2-diaminocyclohexane-malonatoplatinum (II), diaminocarboxylatoplatinum (EPA 296321), trans-(D,1)-1,2-diaminocyclohexa-ne carrier ligand-bearing platinum analogues, aminoalkylaminoanthraquinone-deri-ved cisplatin analogues, spiroplatin, carboplatin, iproplatin and JM40 platinum analogues, bidentate tertiary diamine-containing cisplatinum derivatives, platinum(II), platinum(IV), cis-diammine (1,1-cyclobutanedicarboxylato-) platinum(II) (carboplatin, JM8) and ethylenediammine-malonatoplatinum(II) (JM40), JM8 and JM9 cisplatin analogues, (NPr4)2((PtCL4).cis-(PtC12-(NH2Me)2)), aliphatic tricarboxylic acid platinum complexes (EPA 185225), cis-dichloro(amino acid) (tert-butylamine)platinum-(II) complexes; 4-hydroperoxycylcophosphamide, acyclouridine cyclophosphamide derivatives, 1,3,2-dioxa- and -oxazaphosphorinane cyclophosphamide analogues, C5-substituted cyclophosphamide analogues, tetrahydrooxazine cyclophosphamide analogues, phenyl ketone cyclophosphamide analogues, phenylketophosphamide cyclophosphamide analogues, ASTA Z-7557 cyclophosphamide analogues, 3-(1-oxy-2,2,6,6-tetramethyl-4-piperidinyl)cy-clophosphamide, 2-oxobis(2-β-chloroethylamino)-4-,6-dimethyl-1,3,2-oxazaphosphorinan-e cyclophosphamide, 5-fluoro- and 5-chlorocyclophosphamide, cis- and trans-4-phenylcyclophosphamide, 5-bromocyclophosphamide, 3,5-dehydrocyclophosphamide, 4-ethoxycarbonyl cyclophosphamide analogues, arylaminotetrahydro-2H-1,3,2-oxazaphosphorine 2-oxide cyclophosphamide analogues, NSC-26271 cyclophosphamide analogues, benzo annulated cyclophosphamide analogues, 6-trifluoromethylcyclophosphamide, 4-methylcyclophosphamide and 6-methycyclophosphamide analogues; FCE 23762 doxorubicin derivative, annamycin, ruboxyl, anthracycline disaccharide doxorubicin analogue, N-(trifluoroacetyl)doxorubicin and 4′-O-acetyl-N-(trifluoroacetyl)-doxorubicin, 2-pyrrolinodoxorubicin, disaccharide doxorubicin analogues, 4-demethoxy-7-O-(2,6-dideoxy-4-O-(2,3,6-trideoxy-3-amino-α-L-lyxo-h-exopyranosyl)-α-L-lyxo-hexopyranosyl) adriamicinone doxorubicin disaccharide analog, 2-pyrrolinodoxorubicin, morpholinyl doxorubicin analogues, enaminomalonyl-β-alanine doxorubicin derivatives, cephalosporin doxorubicin derivatives, hydroxyrubicin, methoxymorpholino doxorubicin derivative, (6-maleimidocaproyl)hydrazone doxorubicin derivative, N-(5,5-diacetoxypent-1-yl) doxorubicin, FCE 23762 methoxymorpholinyl doxorubicin derivative, N-hydroxysuccinimide ester doxorubicin derivatives, polydeoxynucleotide doxorubicin derivatives, morpholinyl doxorubicin derivatives (EPA 434960), mitoxantrone doxorubicin analogue, AD198 doxorubicin analogue, 4-demethoxy-3′-N-trifluoroacetyldoxorubicin, 4′-epidoxorubicin, alkylating cyanomorpholino doxorubicin derivative, deoxydihydroiodooxorubicin (EPA 275966), adriblastin, 4′-deoxydoxorubicin, 4-demethyoxy-4′-o-methyldoxorubicin, 3′-deamino-3′-hydroxydoxorubicin, 4-demethyoxy doxorubicin analogues, N-L-leucyl doxorubicin derivatives, 3′-deamino-3′-(4-methoxy-1-piperidinyl) doxorubicin derivatives (U.S. Pat. No. 4,314,054), 3′-deamino-3′-(4-mortholinyl) doxorubicin derivatives (U.S. Pat. No. 4,301,277), 4′-deoxydoxorubicin and 4′-o-methyldoxorubicin, aglycone doxorubicin derivatives, SM 5887, MX-2,4′-deoxy-13(S)-dihydro-4′-iododoxorubicin (EP 275966), morpholinyl doxorubicin derivatives (EPA 434960), 3′-deamino-3′-(4-methoxy-1-piperidi-nyl) doxorubicin derivatives (U.S. Pat. No. 4,314,054), doxorubicin-14-valerate, morpholinodoxorubicin (U.S. Pat. No. 5,004,606), 3′-deamino-3′-(3′-cyano-4″-morpholinyl doxorubicin; 3′-deamino-3′-(3″-cyano-4″-morpholinyl)-13-dihydroxorubicin; (3′-deamino-3′-(3″-cyano-4″-morpholinyl) daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl) doxorubicin derivatives (U.S. Pat. No. 4,314,054) and 3-deamino-3-(4-morpholinyl) doxorubicin derivatives (U.S. Pat. No. 4,301,277); 4,5-dimethylmisonidazole, azo and azoxy misonidazole derivatives; RB90740; 6-bromo and 6-chloro-2,3-dihydro-1,4-benzothi-azines nitrosourea derivatives, diamino acid nitrosourea derivatives, amino acid nitrosourea derivatives, 3′,4′-didemethoxy-3′,4′-dio-xo-4-deoxypodophyllotoxin nitrosourea derivatives, ACNU, tertiary phosphine oxide nitrosourea derivatives, sulfamerizine and sulfamethizole nitrosourea derivatives, thymidine nitrosourea analogues, 1,3-bis(2-chloroethyl)-1-nitrosourea, 2,2,6,6-tetramethyl-1-oxopiperidiunium nitrosourea derivatives (U.S.S.R. 1261253), 2- and 4-deoxy sugar nitrosourea derivatives (U.S. Pat. No. 4,902,791), nitroxyl nitrosourea derivatives (U.S.S.R. 1336489), fotemustine, pyrimidine (II) nitrosourea derivatives, CGP 6809, B-3839, 5-halogenocytosine nitrosourea derivatives, 1-(2-chloroethyl)-3-isobu-tyl-3-(β-maltosyl)-1-nitrosourea, sulfur-containing nitrosoureas, sucrose, 6-((((2-chloroethyl)nitrosoamino-)carbonyl)amino)-6-deoxysucrose (NS-1C) and 6′-((((2-chloroethyl)nitrosoamino)carbonyl)amino)-6′-deoxysucrose (NS-1D) nitrosourea derivatives, CNCC, RFCNU and chlorozotocin, CNUA, 1-(2-chloroethyl)-3-isobutyl-3-β-maltosyl)-1-nitrosourea, choline-like nitrosoalkylureas, sucrose nitrosourea derivatives (JP 84219300), sulfa drug nitrosourea analogues, DONU, N,N′-bis(N-(2-chloroethyl)-N-nitrosocarbamoyl)cystamine (CNCC), dimethylnitrosourea, GANU, CCNU, 5-aminomethyl-2′-deoxyuridine nitrosourea analogues, TA-077, gentianose nitrosourea derivatives (JP 82 80396), CNCC, RFCNU, RPCNU AND chlorozotocin (CZT), thiocolchicine nitrosourea analogues, 2-chloroethyl-nitrosourea, ACNU, (1-(4-amino-2-methyl-5-pyrimidinyl)methyl-3-(2-chloroethyl)-3-nitrosourea hydrochloride), N-deacetylmethyl thiocolchicine nitrosourea analogues, pyridine and piperidine nitrosourea derivatives, methyl-CCNU, phensuzimide nitrosourea derivatives, ergoline nitrosourea derivatives, glucopyranose nitrosourea derivatives (JP 78 95917), 1-(2-chloroethyl)-3-cyclohexyl-1-nitrosourea, 4-(3-(2-chloroethyl)-3-nitrosoureid-o)-1-cis-cyclohexanecarboxylic acid, RPCNU (ICIG 1163), IOB-252, 1,3-bis(2-chloroethyl)-1-nitrosourea (BCNU), 1-tetrahydroxycyclopentyl-3-nitroso-3-(2-chloroethyl)-urea (U.S. Pat. No. 4,039,578), d-1-1-(β-chloroethyl)-3-(2-oxo-3-hexahydroazepinyl)-1-nitrosourea (U.S. Pat. No. 3,859,277) and gentianose nitrosourea derivatives (JP 57080396); 6-S-aminoacyloxymethyl mercaptopurine derivatives, 6-mercaptopurine (6-MP), 7,8-polymethyleneimidazo-1,3,2-diazaph-osphorines, azathioprine, methyl-D-glucopyranoside mercaptopurine derivatives and s-alkynyl mercaptopurine derivatives; indoline ring and a modified ornithine or glutamic acid-bearing methotrexate derivatives, alkyl-substituted benzene ring C bearing methotrexate derivatives, benzoxazine or benzothiazine moiety-bearing methotrexate derivatives, 10-deazaminopterin analogues, 5-deazaminopterin and 5,10-dideazaminopterin methotrexate analogues, indoline moiety-bearing methotrexate derivatives, lipophilic amide methotrexate derivatives, L-threo-(2S,4S)-4-fluoro-glutamic acid and DL-3,3-difluoroglutamic acid-containing methotrexate analogues, methotrexate tetrahydroquinazoline analogue, N-(ac-aminoacyl) methotrexate derivatives, biotin methotrexate derivatives, D-glutamic acid or D-erythrou, threo-4-fluoroglutamic acid methotrexate analogues, β,γ-methano methotrexate analogues, 10-deazaminopterin (10-EDAM) analogue, γ-tetrazole methotrexate analogue, N-(L-α-aminoacyl) methotrexate derivatives, meta and ortho isomers of aminopterin, hydroxymethylmethotrexate (DE 267495), γ-fluoromethotrexate, polyglutamyl methotrexate derivatives, gem-diphosphonate methotrexate analogues (WO 88/06158), α- and γ-substituted methotrexate analogues, 5-methyl-5-deaza methotrexate analogues (U.S. Pat. No. 4,725,687), N.delta.-acyl-N α-(4-amino-4-deoxypteroyl)-L-ornithine derivatives, 8-deaza methotrexate analogues, acivicin methotrexate analogue, polymeric platinol methotrexate derivative, methotrexate-γ-dimyristoylphophatidylethanolamine, methotrexate polyglutamate analogues, poly-γ-glutamyl methotrexate derivatives, deoxyuridylate methotrexate derivatives, iodoacetyl lysine methotrexate analogue, 2,omega.-diaminoalkanoid acid-containing methotrexate analogues, polyglutamate methotrexate derivatives, 5-methyl-5-deaza analogues, quinazoline methotrexate analogue, pyrazine methotrexate analogue, cysteic acid and homocysteic acid methotrexate analogues (U.S. Pat. No. 4,490,529), γ-tert-butyl methotrexate esters, fluorinated methotrexate analogues, folate methotrexate analogue, phosphonoglutamic acid analogues, poly (L-lysine) methotrexate conjugates, dilysine and trilysine methotrexate derivates, 7-hydroxymethotrexate, poly-γ-glutamyl methotrexate analogues, 3′,5′-dichloromethotrexate, diazoketone and chloromethylketone methotrexate analogues, 10-propargylaminopterin and alkyl methotrexate homologs, lectin derivatives of methotrexate, polyglutamate methotrexate derivatives, halogentated methotrexate derivatives, 8-alkyl-7,8-dihydro analogues, 7-methyl methotrexate derivatives and dichloromethotrexate, lipophilic methotrexate derivatives and 3′,5′-dichloromethotrexate, deaza amethopterin analogues, MX068 and cysteic acid and homocysteic acid methotrexate analogues (EPA 0142220); N3-alkylated analogues of 5-fluorouracil, 5-fluorouracil derivatives with 1,4-oxaheteroepane moieties, 5-fluorouracil and nucleoside analogues, cis- and trans-5-fluoro-5,6-dihydro-6-alkoxyuracil, cyclopentane 5-fluorouracil analogues, A-OT-fluorouracil, N4-trimethoxybenzoyl-5′-deoxy-5-fluoro-cytidine and 5′-deoxy-5-fluorouridine, 1-hexylcarbamoyl-5-fluorouracil, B-3839, uracil-1-(2-tetrahydrofuryl)-5-fluorouracil, 1-(2′-deoxy-2′-fluoro-β-D-arabinofuranosyl)-5-fl-uorouracil, doxifluridine, 5′-deoxy-5-fluorouridine, 1-acetyl-3-O-toluoyl-5-fluorouracil, 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680); 4′-epidoxorubicin; N-substituted deacetylvinblastine amide (vindesine) sulfates; and Cu(II)-VP-16 (etoposide) complex, pyrrolecarboxamidino-bearing etoposide analogues, 40-amino etoposide analogues, γ-lactone ring-modified arylamino etoposide analogues, N-glucosyl etoposide analogue, etoposide A-ring analogues, 4′-deshydroxy-4′-methyl etoposide, pendulum ring etoposide analogues and E-ring desoxy etoposide analogues.

In one embodiment, the cytotoxic agent is a small drug molecule (Payne et al., U.S. Pat. No. 7,202,346). In another embodiment, the cytotoxic agent a maytansinoid, an analog of a maytansinoid, a prodrug of a maytansinoid, or a prodrug of an analog of a maytansinoid (U.S. Pat. Nos. 6,333,410; 5,475,092; 5,585,499; 5,846,545; 7,202,346). In another embodiment, the cytotoxic agent may be a taxane (see U.S. Pat. Nos. 6,340,701 & 6,372,738 & 7,202,346) or CC-1065 analog (see U.S. Pat. Nos. 5,846,545; 5,585,499; 5,475,092 & 7,202,346).

In another embodiment, the cytotoxic agent is exemplified by an auristatin, a DNA minor groove binding agent, a DNA minor groove alkylating agent, an enediyne, a duocarmycin, a maytansinoid, and a vinca alkaloid (U.S. Pat. No. 7,662,387).

In a further embodiment, the cytotoxic agent is an anti-tubulin agent (U.S. Pat. No. 7,662,387). In yet another embodiment, the cytotoxic agent is exemplified by dimethylvaline-valine-dolaisoleuine-dolaproine-phenylalanine-p-phenylenediamine (AFP), dovaline-valine-dolaisoleunine-dolaproine-phenylalanine (MMAF), and monomethyl auristatin E (MAE) (U.S. Pat. No. 7,662,387).

Anti-cancer toxins are further exemplified by methotrexate, 5-fluorouracil, cycloheximide, daunomycin, doxorubicin, chlorambucil, trenimon, phenylenediamine mustard, adriamycin, bleomycin, cytosine arabinoside or Cyclophosphamide (U.S. Pat. No. 5,057,13). Further representative examples of anti-cancer toxins include taxanes (e.g., paclitaxel and docetaxel). Etanidazole, Nimorazole, perfluorochemicals with hyperbaric oxygen, transfusion, erythropoietin, BW12C, nicotinamide, hydralazine, BSO, WR-2721, IudR, DUdR, etanidazole, WR-2721, BSO, mono-substituted keto-aldehyde compounds, nitroimidazole, 5-substituted-4-nitroimidazoles, SR-2508, 2H-isoindolediones (U.S. Pat. No. 4,494,547), chiral (((2-bromoethyl)-amino)methyl)-1-nitro-1H-imidazole-1-ethanol (U.S. Pat. No. 5,543,527; U.S. Pat. No. 4,797,397; U.S. Pat. No. 5,342,959), nitroaniline derivatives (U.S. Pat. No. 5,571,845), DNA-affinic hypoxia selective cytotoxins (U.S. Pat. No. 5,602,142), halogenated DNA ligand (U.S. Pat. No. 5,641,764), 1,2,4 benzotriazine oxides (U.S. Pat. No. 5,616,584; U.S. Pat. No. 5,624,925; U.S. Pat. No. 5,175,287), nitric oxide (U.S. Pat. No. 5,650,442), 2-nitroimidazole derivatives (U.S. Pat. No. 4,797,397; U.S. Pat. No. 5,270,330; U.S. Pat. No. 5,270,330; Patent EP 0 513 351 B1), fluorine-containing nitroazole derivatives (U.S. Pat. No. 4,927,941), copper (U.S. Pat. No. 5,100,885), combination modality cancer therapy (U.S. Pat. No. 4,681,091), 5-CldC or (d)H.sub.4U an/or 5-halo-2′-halo-2′-deoxy-cytidine and/or -uridine derivatives (U.S. Pat. No. 4,894,364), platinum complexes (U.S. Pat. No. 4,921,963; Patent EP 0 287 317 A3), fluorine-containing nitroazole (U.S. Pat. No. 4,927,941), benzamide, autobiotics (U.S. Pat. No. 5,147,652), benzamide and nicotinamide (U.S. Pat. No. 5,215,738), acridine-intercalator (U.S. Pat. No. 5,294,715), fluorine-containing nitroimidazole (U.S. Pat. No. 5,304,654, Apr. 19, 1994), hydroxylated texaphyrins (U.S. Pat. No. 5,457,183), hydroxylated compound derivative (Publication Number 011106775 A (Japan), Oct. 22, 1987; Publication Number 01139596 A (Japan), Nov. 25, 1987; Publication Number 63170375 A (Japan)), fluorine containing 3-nitro-1,2,4-triazole (Publication Number 02076861 A (Japan), Mar. 31, 1988), 5-thiotretrazole derivative or its salt (Publication Number 61010511 A (Japan), Jun. 26, 1984), Nitrothiazole (Publication Number 61167616 A (Japan) Jan. 22, 1985), imidazole derivatives (Publication Number 6203767 A (Japan) Aug. 1, 1985; Publication Number 62030768 A (Japan) Aug. 1, 1985; Publication Number 62030777 A (Japan) Aug. 1, 1985), 4-nitro-1,2,3-triazole (Publication Number 62039525 A (Japan), Aug. 15, 1985), 3-nitro-1,2,4-triazole (Publication Number 62138427 A (Japan), Dec. 12, 1985), Carcinostatic action regulator (Publication Number 63099017 A (Japan), Nov. 21, 1986), 4,5-dinitroimidazole derivative (Publication Number 63310873 A (Japan) Jun. 9, 1987), nitrotriazole Compound (Publication Number 07149737 A (Japan) Jun. 22, 1993), cisplatin, doxorubin, misonidazole, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide (Tannock. Journal of Clinical Oncology 14(12):3156-3174, 1996), camptothecin (Ewend et al. Cancer Research 56(22):5217-5223, 1996) and paclitaxel (Tishler et al. Journal of Radiation Oncology and Biological Physics 22(3):613-617, 1992).

In another embodiment, the molecule of interest comprises a therapeutic molecule. The term “therapeutic molecule” refers to a molecule that reduces, delays and/or eliminates undesirable pathologic effects in a cell, tissue, organ and/or animal.

Therapeutic molecules are exemplified by therapeutic sequences (e.g., therapeutic nucleotide sequences and/or the encoded therapeutic polypeptides), which may be homologous or heterologous with respect to the sequences of the target cell into which they are introduced.

Homologous therapeutic sequences are useful for expressing wild-type proteins where it is desirable to, for example, compensate for either insufficient expression of a wild-type protein product in the cell or to bring about expression of a mutant protein product whose biological activity is reduced relative to the wild-type protein.

Heterologous therapeutic sequences are useful in, for example, expressing a mutant protein which is less active, more active, and/or more stable, than the wild-type protein. Alternatively, heterologous therapeutic nucleotide sequences may be used to express a heterologous protein which is derived from a species that is different from the target cell species, such that the expressed heterologous protein complements or supplies a deficient activity in the target cell, thus allowing the latter to resist a pathological process, or else stimulate an immune response.

In one embodiment, the therapeutic nucleotide sequence is a “suicide gene,” i.e., a gene encoding “suicide protein” such as an enzyme that can metabolize a separately administered non-toxic pro-drug into a potent cytotoxin, which can diffuse to and kill neighboring cells. A herpes simplex virus, encoding a thymidine kinase suicide gene, has progressed to phase III clinical trials. The herpes simplex virus thymidine kinase (HSVtk) phosphorylates the pro-drug, gancyclovir, which is then incorporated into DNA, blocking DNA synthesis.

The terms “alter” and “modify” when in reference to the level of any molecule and/or phenomenon refer to an increase and/or decrease.

### DESCRIPTION OF THE INVENTION

The invention provides the breakthrough discovery, isolation, and characterization of pre-malignant hepatocellular carcinoma initiating cells (HICs). The invention further provides methods for isolating hepatocellular carcinoma initiating cells (HICs), methods for using the isolated cells for screening anti-cancer drugs, methods for using HIC markers for the early diagnosis of hepatocellular carcinoma, and methods for the prevention and/or delay of hepatocellular carcinoma by using agents that selectively deplete the number and/or malignant properties of HICs.

Importantly, to date, there is no report of a successful isolation of pre-malignant HIC that generate HCC in transplanted animals from livers with precancerous lesions. Thus, data herein demonstrate that HIC were detected in livers of carcinogen treated or genetically altered mice long before malignant hepatocellular carcinoma (HCC) nodules can be detected. Although foci of morphologically altered hepatocytes (FAH) were described in livers of carcinogen-treated animals and suggested to represent pre-neoplastic lesions⁴⁴⁻⁴⁶, no one has succeeded in isolating such cells and transplanting them to another animal. Indeed, it was questioned whether FAH represent true pre-malignant lesions or a regenerative response to a cytotoxic carcinogen^{9,47}. Notably, precancerous lesions were detected in cirrhotic livers⁴⁸⁻⁵⁰, but there is no conclusive evidence, in the absence of proper isolation procedures, that cells within such lesions have malignant potential.

The invention's hepatocellular carcinoma initiating cells (HIC) are useful in further definition of molecular and phenotypic changes in the progression of normal liver tissue to precancerous lesions and cancer in experimental animal models,

The invention's methods for isolating and characterizing hepatocellular carcinoma initiating cells (HIC) from mouse are useful in further applying these methods to the isolation and characterization of hepatocellular carcinoma initiating cells (HIC) in livers of human individuals, such as individuals suffering from liver diseases that greatly increase HCC risk.

The invention's hepatocellular carcinoma initiating cells (HIC) are useful for early diagnosis of hepatocellular carcinoma detection of true pre-malignant lesions in human liver, as well as early detection of malignant HCC nodules. For example, hepatocellular carcinoma initiating cells (HIC) cell surface markers can be used for development of functional imaging techniques that can reliably distinguish hepatocellular carcinoma (HCC) from benign hepatic lesions.

In addition, the invention's hepatocellular carcinoma initiating cells (HIC) are useful in the prevention and/or treatment of cancer. For example, antibodies that target HIC-specific markers can be used to generate toxic conjugates that eliminate pre-malignant hepatocytes before they progress to aggressive HCC, refractory to conventional anti-cancer agents. The destruction of such pre-malignant lesions by either antibody-toxin conjugates or liposome-mediated delivery of tumoricidal genes would provide an effective prophylactic therapy for HCC, a cancer that is refractory to all currently existing anti-cancer agents. Although HCC stem cells were described, such cells were mainly isolated from established HCC cell lines.

In one embodiment, the invention provides methods for detecting the presence of cancer initiating cells in a tissue, methods for identifying test agent for reducing cancer, and methods for reducing cancer in a subject. The invention's methods are applicable to any cancer, and in particular to liver cancer.

To date, there are no reports of the isolation of liver cancer initiating cells, and in particular, isolation of liver cancer initiating cells before a visible tumor can be detected. The invention provides the discovery of the identification and isolation of cancer initiating cells with altered morphology in livers of mice treated with a carcinogen that induces liver cancer. The inventors developed methods to isolate these cells and demonstrated that they are liver cancer (hepatocellular carcinoma or HCC) initiating cells. The cancer initiating cells were isolated from non-malignant lesions rather than from cancerous masses, and were isolated based both on their morphology and molecular signature.

The inventors have identified cell surface protein markers expressed by liver cancer initiating cells, and provide the discovery that antagonists of these protein marker inhibit liver cancer formation and prevent development of liver cancer in high risk individuals.

These cells are responsible for generation of HCC, the most common form of liver cancer in humans.

The invention also provides the discovery that early detection of these cells in individuals that are at high risk of HCC development will allow the timely administration of drugs that target these cancer initiating cells and prevent the formation of liver cancer.

The inventors also developed a transplant system to monitor the progression of liver cancer initiating cells into overt cancer. This can also be done with human liver cancer initiating cells.

The invention's methods are useful for early detection of cancer, e.g., liver cancer, the third most deadly cancer in the world, early intervention and prevention of cancer, e.g., liver cancer, and screening of therapeutic and preventive targets for cancer, e.g., liver cancer.

The invention is further described under A) Cancer Initiating Cells, B) Hepatocellular carcinoma initiating cells (HICs), C) Methods for isolating hepatocellular carcinoma initiating cells (HICs), D) Methods for identifying HIC markers, E) Use of HICs in cancer diagnosis and/or detection, F) Use of HICs to screen anti-cancer agents, G) Methods for reducing hepatocellular carcinoma initiating cells (HICs) and/or reducing hepatocellular carcinoma, H) Detecting Expression, I) Test Compounds, and J) Administering compounds, K) IKK/NF-κB signaling pathway in HCC development, L) STAT3 in liver cancer, M) Crosstalk between IKK/NF-κB and STAT3 in liver cancer, N) Discussion of results in Examples 12-16, O) Methods for determining progression of HICs into HCCs.

### A. Cancer Initiating Cells

The invention provides the discovery of cancer initiating cells, and methods for their identification, such as for liver cancer initiating cells. The inventors have isolated for the first time cancer initiating cells from pre-malignant liver. The inventors have also identified critical cell surface proteins expressed by these cells, antagonists of which should inhibit liver cancer formation.

“Cancer initiating cell” and “C-IC” interchangeably refer to a cell that is capable of producing a cancer cell when introduced into a suitable host animal such as an immunodeficient host animal. In one embodiment, the cancer initiating cell is not a cancer cell (such as a cell that lacks morphological characteristics of and/or biochemical markers of a cancer cell). Cancer initiating cell includes, without limitation, a stem cell and a progenitor cell.

A “stem cell” is an undifferentiated (unspecialized) cell that is found in a differentiated (specialized) tissue, and that has the capacity to replicate indefinitely, and to differentiate to become specialized to yield specialized cell types of the tissue from which it originated. Stem cells are distinguished from progenitor cells in that stem cells can replicate indefinitely, whereas progenitor cells can only divide a limited number of times.

A “progenitor cell,” like a stem cell, has the capacity to differentiate into a specific type of cell. In contrast to a stem cell, a progenitor cell has already begun the process of differentiating into a “target” cell, and can only divide a limited number of times.

Tumor cells are very heterogeneous. It is suggested that there exists a small subset of cancer cells, termed cancer stem cells (CSC), that gives rise to all cell types found in a particular cancer sample (1, 2). These cells are responsible for initiating and maintaining the disease through their ability to proliferate and self-renew (1, 2). In another word, CSCs are cancer cells (found within tumors) that are tumorigenic (tumor-forming), in contrast to other non-tumorigenic cancer cells.

The existence of cancer initiating cells was first demonstrated in human acute myeloid leukemia by John E. Dick's group (14, 15). These leukemic cells, which were defined by specific markers of CD34+CD38−, can reproduce the disease when transplanted into immunodeficient mice. Putative cancer initiating cells were subsequently identified in other types of human cancers, such as breast cancer (16), colon cancer (17), melanoma (18), and liver cancer (19). In contrast to the invention, all these cancer initiating cells are isolated from existing malignant tumors.

The existence of cancer stem/initiating cells was first demonstrated in human acute myeloid leukemia (3, 4). These leukemic cells, which were defined by specific markers of CD34+CD38−, can reproduce the disease when transplanted into immunodeficient mice. Putative cancer stem/initiating cells were subsequently identified in other types of human cancers, such as breast cancer (5), colon cancer (6), melanoma (7), and liver cancer (8). CSCs from different cancer types are all capable of self-renewal and forming tumors with high efficiency when transplanted to nude mice (1).

The existence of CSCs has several implications in terms of future cancer treatment and therapies. These include disease identification, selective drug targets, prevention of metastasis, and development of new intervention strategies. Normal somatic stem cells are naturally resistant to chemotherapeutic agents—they have various pumps (such as MDR) that pump out drugs, DNA repair proteins and they also have a slow rate of cell turnover (chemotherapeutic agents naturally target rapidly replicating cells). CSCs may also express proteins that would increase their resistance towards chemotherapeutic agents. If current treatments of cancer do not properly destroy enough CSCs, these surviving CSCs then repopulate the tumor, causing relapse (9). By selectively targeting CSCs, it would be possible to treat patients with aggressive, non-resectable tumors, as well as preventing the tumor from metastasizing. The hypothesis suggests that upon CSC elimination, cancer would regress due to differentiation and/or cell death (9).

Although cancer stem cells are potentially very promising targets for therapeutic drugs, little success has been documented despite near 2 decades of extensive studies in this direction. Cancer stem cells described so far in the literature are all isolated from malignant tumors and they are cancer cells themselves. One important feature shared by all cancer cells is that they are genetically instable and are prone to continuous genetic alterations spontaneously or upon environmental pressure. Therefore, current cancer therapies may kill the majority of cancer cells/cancer stem cells, at the same time they also represent a selection pressure driving the development of new drug-resistant cancer cells. Now for the first time, the inventors have isolated cancer initiating cells (C-IC) from pre-malignant liver. These cells are not cancer cells yet but will eventually give rise to malignant tumors without proper intervention. These C-ICs are likely more responsive to current or future cancer therapies than cancer stem cells found in existing malignant tumors. Most importantly, by targeting C-ICs in the early stages of cancer development, the inventors have the promise to cure cancer even before it actually appears. This is extremely important because it is estimated that more than 530 million people in the world were chronically infected with hepatitis virus B or C or both. These people are the high-risk population for liver cancer and may harbor C-ICs already in their livers, and therefore are all potential market for future therapies targeting C-ICs.

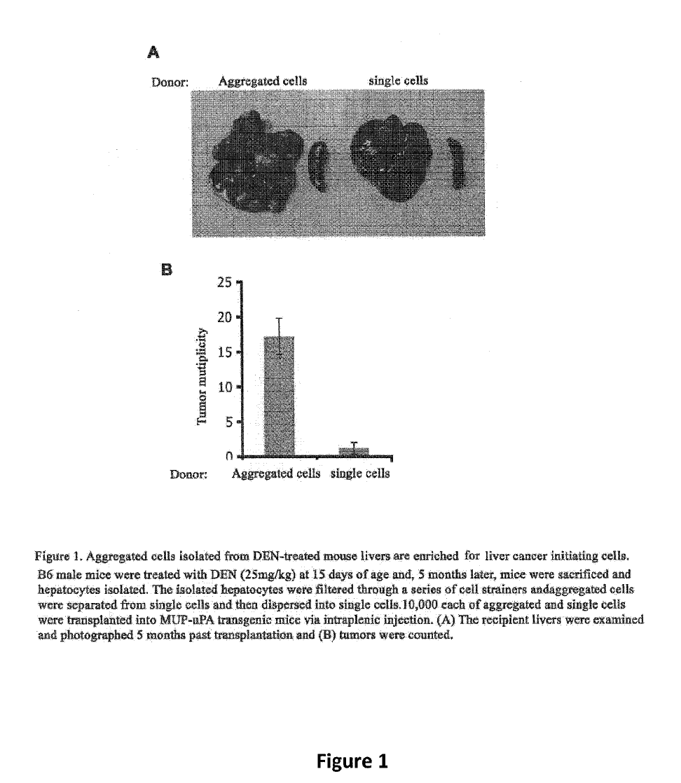

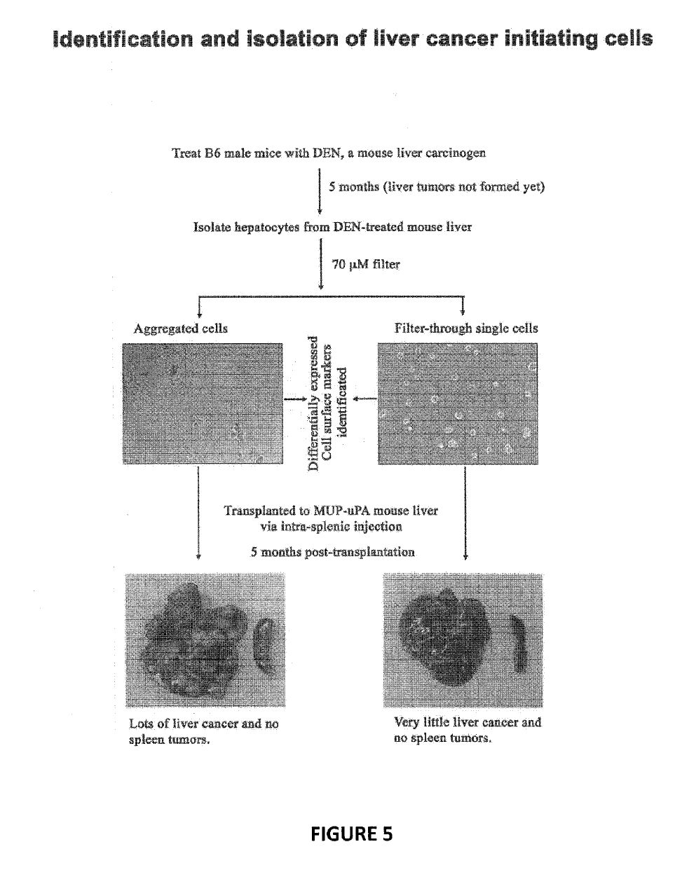

Another obstacle to effectively targeting current cancer stem cells is the prior art's inability to identify critical targetable molecules inside cancer stem cells. The way people identify and isolate cancer stem cells right now is solely relied on differentially expressed cell surface markers. Most of these cell surface markers are just a marker and not targetable. Furthermore, not a single marker is found common for all cancer stem cells. Now the inventors have developed a functional approach allowing us to isolate cancer initiating cells based on their morphology (aggregates), which is not possible before. The inventors find that cell aggregates begin to appear in DEN (carcinogen)-treated liver, but not in normal control liver, as early as 3-5 months past DEN treatment (FIG. 5). The inventors have developed a novel transplant system and, with this powerful system, the inventors have successfully showed that these aggregates contain enriched cancer initiating cells giving rise to overt liver cancer (FIG. 1). These findings provide us with much-needed clues about targetable pathways for targeting cancer stem/initiating cells, that is, cell adhesion signaling pathway. We may effectively target cancer initiating cells by targeting cell adhesion molecules that are responsible for the formation of cell aggregates.

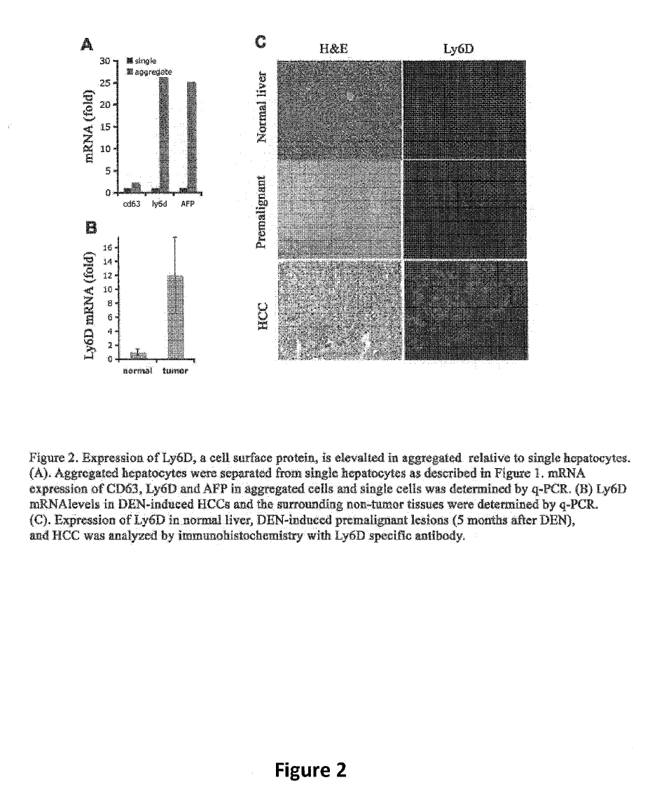

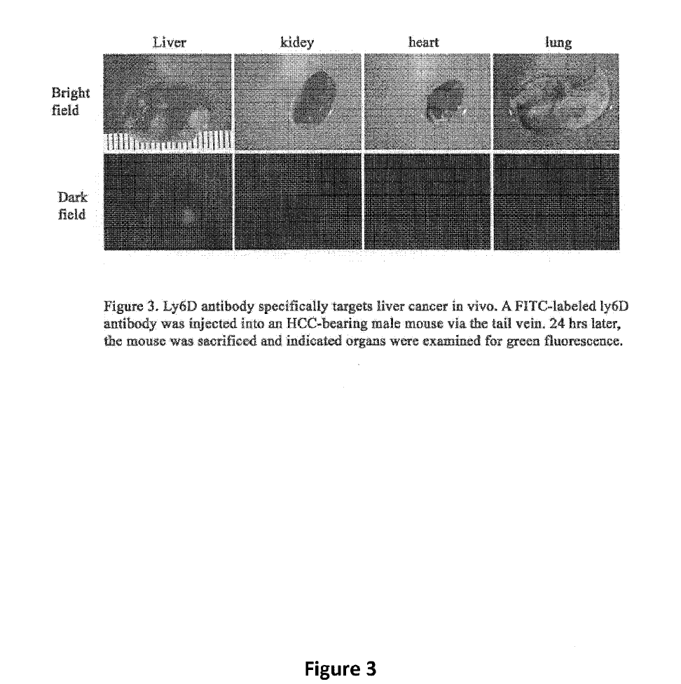

Using the above strategy, the inventors have identified a list of cell adhesion molecules that are significantly up-regulated in cell aggregates than control single cells. One of these molecules is Ly6D (FIG. 2). The inventors have also shown, by both real-time PCR and immunofluorescence staining, that Ly6D is highly upregulated in liver pre-malignant lesions and liver cancer relative to normal liver (FIG. 2). The inventors also used a commercially available Ly6D antibody that specifically homes to liver cancer when injected into liver cancer-bearing mouse (FIG. 3). The results in FIG. 3 demonstrate that Ly6D is a targetable molecule for liver cancer cells and/or for liver cancer initiating cells.

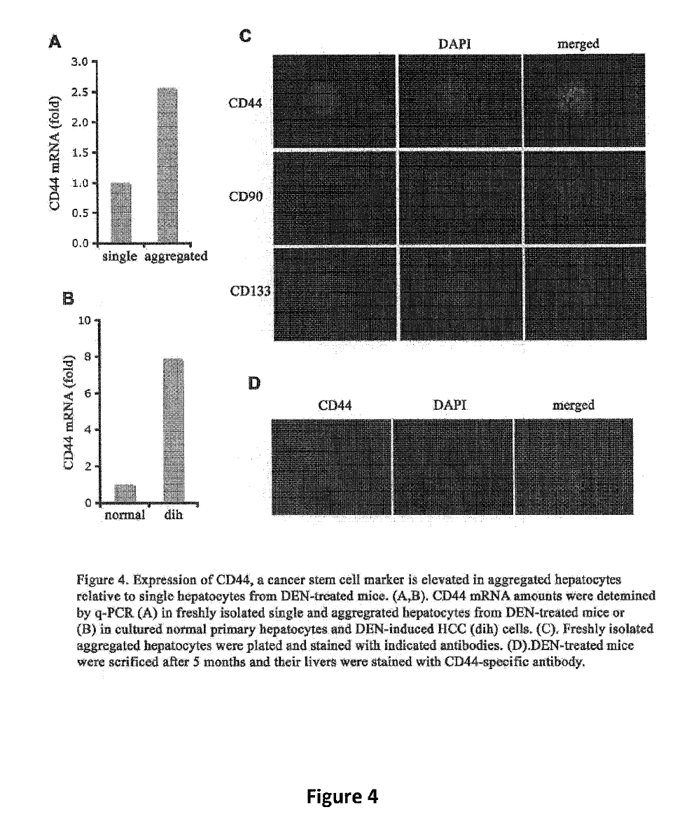

Another interesting molecule on our list is CD44, a known cell marker for cancer stem cells in some solid cancers (11). CD44 is found up-regulated in cell aggregates in DEN-treated mouse livers and in DEN-induced liver cancer (FIG. 4). It has also been shown in the literature that forced expression of CD44 in fibroblast results in cell aggregates (12, 13). The inventors provide CD44 as a target for compounds that reduce liver cancer initiating cells.

### B. Hepatocellular Carcinoma Initiating Cells (HICs)

The invention provides an isolated population of mammalian hepatocellular carcinoma initiating cells (HICs).

The invention's isolated HICs are useful in methods for identifying HIC marker genes and/or marker proteins and/or HIC marker antigens, for the diagnosis and early detection (e.g. by histochemical detection and imaging approaches) of hepatocellular carcinoma.

The invention's isolated HICs are also useful in methods for identifying agents that reduce hepatocellular carcinoma initiating cells (HICs), for the prevention of and/or delaying development of and/or treatment of hepatocellular carcinoma.

Data herein demonstrate that HIC were isolated based on their ability to form collagenase-resistant aggregates and their tumorigenic potential was demonstrated by transplantation into mice whose livers undergo persistent compensatory proliferation that allows HIC to progress into fully malignant HCC. Data herein also shows that a comparison of the gene expression profile of carcinogen-induced HIC to that of non-transformed hepatocytes demonstrates that HICs exhibit certain similarities to bipotential hepatobiliary progenitors and oval cells.

The invention also provides a composition comprising the isolated population of mammalian hepatocellular carcinoma initiating cells (HICs) described herein.

### C. Methods for Isolating Hepatocellular Carcinoma Initiating Cells (HICs)

The invention provides a method for producing the isolated population of mammalian hepatocellular carcinoma initiating cells (HICs), comprising a) treating liver tissue from a mammalian subject with collagenase to produce a composition comprising a population of aggregated hepatocellular cells and a population of non-aggregated hepatocellular cells, and b) isolating the population of aggregated hepatocellular cells from the composition, thereby producing an isolated population of mammalian hepatocellular carcinoma initiating cells (HICs).

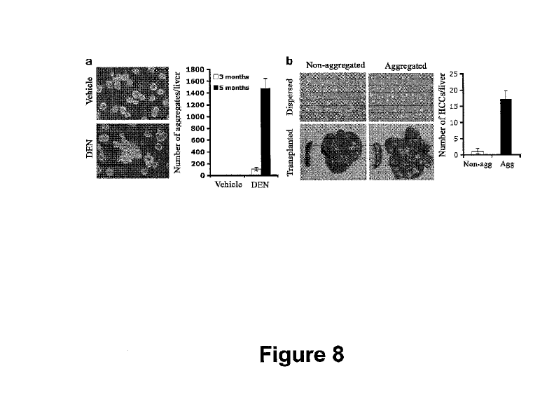

Data herein (Example 2, FIG. 8) show that instead of yielding uniform suspensions of single hepatocytes as it does in PBS-injected mice, collagenase digestion of DEN-treated mouse livers generated a population of collagenase-resistant, tightly-packed aggregates of small hepatocytes in addition to well dispersed, single hepatocytes (FIG. 8a). The aggregates were almost 20-fold more potent in initiating HCC than non-aggregated hepatocytes from the same liver (FIG. 8b).

While not intending to limit the subject, in one embodiment, the mammalian subject is a mouse. In one embodiment, the mouse is selected from the group of a DEN-treated mouse, a mouse that lacks expression of TAK1, and a mouse that lacks expression of TAK1 and p38.

In another embodiment, the mammalian subject is human. In a particular embodiment, the human mammalian subject is at risk of developing liver hepatocellular carcinoma (HCC). In another embodiment, the human mammalian subject has liver hepatocellular carcinoma (HCC).

The invention also provides an isolated population of mammalian hepatocellular carcinoma initiating cells (HICs) produced by the methods described herein

### D. Methods for Identifying HIC Markers

The invention provides a method for identifying a HIC marker gene, comprising determining the level of expression of a gene in a) an isolated population of mammalian hepatocellular carcinoma initiating cells (HICs), and b) control non-cancerous cells, wherein an altered level of gene expression in the HICs compared to the control cells identifies the gene as a HIC marker gene.

The invention's methods are useful for the diagnosis and early detection (e.g. by histochemical detection and imaging approaches) of hepatocellular carcinoma.

The identification and isolation of pre-malignant hepatocytes (HICs) allows further definition of molecular and phenotypic changes in the progression of normal liver tissue to precancerous lesions and cancer in experimental animal models.

Furthermore, the methodology used to define pre-malignant HIC in mouse models is applicable to the identification of such cells in livers of human individuals suffering from liver diseases that greatly increase HCC risk. As a first step towards this goal, we will examine expression of mouse HIC markers in precancerous human livers and correlate their presence with HCC risk.

In one embodiment, the HIC marker gene encodes an HIC cell surface marker antigen.

In a further embodiment, the control cells are selected from hepatic oval cells and hepatic normal cells.

E. Use of HICs in Cancer Diagnosis and/or Detection

The invention provides methods useful for the diagnosis and early detection of hepatocellular carcinoma. The invention also provides methods that are also useful for prevention of and/or delaying development of and/or treatment of hepatocellular carcinoma.

Thus, in one embodiment, the invention provides a method for detecting the presence of hepatocellular carcinoma initiating cells (HICs) in a sample, comprising a) introducing an isolated population of mammalian hepatocellular carcinoma initiating cells (HICs) into a mammalian host mammalian subject to produce a treated subject, and b) detecting hepatocellular cancer (HCC) in the treated subject, thereby detecting the presence of hepatocellular carcinoma initiating cells (HICs) in the sample. In one embodiment, the sample comprises liver tissue.

The invention also provides a method for detecting the presence of hepatocellular carcinoma initiating cells (HICs) in a sample, comprising detecting in the sample a HIC marker gene. In one embodiment, the detecting step comprises determining an altered level of expression of the HIC marker gene in the sample compared to the level of expression of the HIC marker gene in a control sample. In one embodiment, the control sample is selected from hepatic oval cell sample and hepatic normal cell sample.

In a particular embodiment, the HIC marker gene encodes an HIC cell surface marker antigen, and wherein the detecting comprises determining an altered level of expression of the HIC cell surface marker antigen in the sample compared to the level of expression of the HIC cell surface marker antigen in a control sample, such as hepatic oval cell sample and hepatic normal cell sample. In one embodiment, the sample comprises liver tissue.

### F. Use of HICs to Screen Anti-Cancer Agents

The invention provides a method for identifying a test agent as reducing hepatocellular carcinoma initiating cells (HICs), comprising a) contacting i) an isolated population of mammalian hepatocellular carcinoma initiating cells (HICs), with a test agent, and b) detecting at least one of i) reduced number of the HICs, and ii) reduced malignancy of the HICs, wherein the detecting identifies the test agent as reducing hepatocellular carcinoma initiating cells (HICs).

In one embodiment, the test agent is selected from the group consisting of anti-cancer cytotoxin, antibody that specifically binds to a HIC cell surface marker antigen, RNA interference sequence that specifically binds to mRNA that encodes a HIC marker protein, and antisense sequence that encodes a HIC marker protein. In a particular embodiment, the anti-cancer cytotoxin comprises a nucleotide sequence encoding herpes simplex virus thymidine kinase (HSVtk). To determine the efficiency of HSVtk DNA delivery and expression, livers of treated mice are stained with antibodies to HSVtk to make sure the viral enzyme is efficiently expressed in the majority of HIC and preferably only in HIC. These experiments will serve as a blueprint for preparation of similar reagents for targeting human HIC. HSVtk may be expressed from the AFP promoter/enhancer which is active only in pre-neoplastic and HCC cells, to avoid HSVtk expression in normal hepatocytes. (Example 9).

In another embodiment, the antibody that specifically binds to a HIC cell surface marker antigen is selected from the group of antibody that specifically binds to CD44, and antibody that specifically binds to CD44v6.

In an alternative embodiment, the test agent is covalently linked to an antibody that specifically binds to a HIC cell surface marker antigen. Antibodies that target HIC-specific markers can be used to generate toxic conjugates that eliminate pre-malignant hepatocytes before they progress to aggressive HCC, refractory to conventional anti-cancer agents. In one embodiment, monoclonal antibodies to CD44 or other HIC specific antigens are coupled to DM1 as described (Siddiquee et al., Proc Natl Acad Sci USA 2007; 104:7391-6; Lin et al., Oncogene 2009; 28:961-72) (Example 9)

In a further embodiment, the test agent further comprises a liposome. For example, the AFP-HSVtk construct may be delivered to HIC via liposomes, composed of synthetic cationic lipid bilayers which can be complexed with plasmid DNA using established procedures (Mohr et al., Hum Gene Ther 12, 799-809 (2001); Siwak et al., Clin. Cancer Res., 8: 1172-1181, 2002). Clin Cancer Res 8, 955-956 (2002). In a particular embodiment, the liposome further comprises an antibody that specifically binds to a HIC cell surface marker antigen. This may be desirable in order to target liposomes primarily to HIC, by containing a monoclonal antibody to an HIC cell surface marker, for instance CD44 (Example 9).

G. Methods for Reducing Hepatocellular Carcinoma Initiating Cells (HICs) and/or Reducing Hepatocellular Carcinoma,

The invention provides a method for reducing hepatocellular carcinoma initiating cells (HICs) in a mammalian subject comprising administering to a subject in need thereof a therapeutic amount of an agent that reduces hepatocellular carcinoma initiating cells (HICs).

The invention's methods are useful for prevention of and/or delaying development of and/or treatment of hepatocellular carcinoma.

In one embodiment, the method further comprises detecting at least one of a) reduced number of the HICs, and b) reduced malignancy of the HICs. In another embodiment, the method further comprises detecting reduced hepatocellular carcinoma (HCC) in the subject.

### H. Detecting Expression

Expression levels of the invention's proteins may be determined using antibodies that specifically bind to the protein. Such antibodies may be employed in Western blots, “sandwich” immunoassays such as ELISA (enzyme-linked immunosorbant assay), and ELISpot (enzyme-linked immunosorbent spot assay), immunoradiometric assays, gel diffusion precipitin reactions, immunodiffusion assays, in situ immunoassays (using colloidal gold, enzyme or radioisotope labels, for example), Western Blots, etc.), complement fixation assays, immunofluorescence assays, immunohistochemical staining, protein A assays, and immunoelectrophoresis assays, etc.

Alternatively, or in addition, expression levels of the invention's proteins may be determined by determining the level of mRNA that encodes the invention's protein. This may be accomplished using know methods such as Northern blot hybridization, reverse transcription polymerase chain reaction, in situ hybridization to RNA, etc.

For example, in Northern blot analysis, RNA is isolated from cells and electrophoresed on agarose gels to fractionate the RNA according to size followed by transfer of the RNA from the gel to a solid support, such as nitrocellulose or a nylon membrane. The immobilized RNA is then probed with a labeled oligo-deoxyribonucleotide probe or DNA probe to detect RNA species complementary to the probe used. Northern blots are a standard tool of molecular biologists.

The terms “reverse transcription polymerase chain reaction” and “RT-PCR” refer to a method for reverse transcription of an RNA sequence to generate a mixture of cDNA sequences, followed by increasing the concentration of a desired segment of the transcribed cDNA sequences in the mixture without cloning or purification. Typically, RNA is reverse transcribed using one or two primers prior to PCR amplification of the desired segment of the transcribed DNA using two primers.

### I. Test Compounds

The terms “test compound,” “compound,” “agent,” “test agent,” “molecule,” and “test molecule,” as used herein, refer to any type of molecule (for example, a peptide, polypeptide, vaccine, antibody, nucleic acid, nucleic acid sequence, carbohydrate, saccharide, polysaccharide, lipid, organic molecule, inorganic molecule, etc.) obtained from any source (for example, plant, animal, and environmental source, etc.), or prepared by any method (for example, purification of naturally occurring molecules, chemical synthesis, and genetic engineering methods, etc.).

A test compound may have a known, or unknown, structure and/or composition. Examples of test compounds that have unknown compositions include cell extracts, tissue extracts, growth medium in which prokaryotic, eukaryotic, and archaebacterial cells have been cultured, fermentation broths, protein expression libraries, DNA libraries, and the like.

The “test compound,” can be synthetic, naturally occurring, or a combination thereof. A synthetic test compound can be a member of a library of test compounds (e.g., a combinatorial chemical library). Methods for making these libraries of compounds are known in the art, such as methods for preparing oligonucleotide libraries (Gold et al., U.S. Pat. No. 5,270,163, incorporated by reference); peptide libraries (Koivunen et al. J. Cell Biol., 124: 373-380 (1994)); peptidomimetic libraries (Blondelle et al., Trends Anal. Chem. 14:83-92 (1995)) oligosaccharide libraries (York et al., Carb. Res. 285:99-128 (1996); Liang et al., Science 274:1520-1522 (1996); and Ding et al., Adv. Expt. Med. Biol. 376:261-269 (1995)); lipoprotein libraries (de Kruif et al., FEBS Lett., 399:232-236 (1996)); glycoprotein or glycolipid libraries (Karaoglu et al., J. Cell Biol. 130:567-577 (1995)); or chemical libraries containing, for example, drugs or other pharmaceutical agents (Gordon et al., J. Med. Chem. 37:1385-1401 (1994); Ecker and Crook, Bio/Technology 13:351-360 (1995), U.S. Pat. No. 5,760,029, incorporated by reference). Additional examples of methods for the synthesis of molecular libraries can be found in the art, for example in: DeWitt et al., Proc. Natl. Acad. Sci. U.S.A. 90:6909 (1993); Erb et al., Proc. Nad. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33.2059 (1994); Carell et al., Angew. Chem. Int. Ed. Engl. 33:2061 (1994); and Gallop et al., J. Med. Chem. 37:1233 (1994).

A synthetic test compound may be a member of a biological library or peptoid library (i.e., library of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 (1994)). The biological library and peptoid library are particularly suited for use with peptide libraries.

In addition, a synthetic test compound may be a member of a spatially addressable parallel solid phase library or solution phase library, of a synthetic library that uses methods such as a deconvolution method, a ‘one-bead one-compound’ library method, affinity chromatography selection. These methods are particularly suited to peptide libraries, non-peptide oligomer libraries, and small molecule libraries of compounds (Lam (1997) Anticancer Drug Des. 12:145).

Libraries of diverse molecules also can be obtained from commercial sources, e.g., Brandon Associates (Merrimakc, N.H.) and Aldrich Chemical Co (Milwaukee, Wis.).

A naturally occurring test compound can be a component of a cellular extract or bodily fluid (e.g., urine, blood, tears, sweat, or saliva). A naturally occurring test compound may be obtained by extraction and/or purification of commercially available libraries of bacterial, fungal, plant, and animal extracts.

A test compound includes both known therapeutic compounds, and potentially therapeutic compounds. An agent can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The compounds of the invention may be administered before, concomitantly with, and/or after manifestation of one or more symptoms of cancer. The term “concomitant” when in reference to the relationship between administration of a compound and a disease symptoms means that administration occurs at the same time as, or during, manifestation of the disease symptom. Also, the invention's agents may be administered before, concomitantly with, and/or after administration of another type of drug or therapeutic procedure (e.g., surgery, chemotherapy, radiotherapy, etc.).

The following are exemplary compounds that may be useful in the invention's methods, such as for reducing expression of one or both of Ly6D protein and CD44 protein, and/or for reducing the biological activity of one or both of Ly6D protein and CD44 protein.

1. Antibodies

In one embodiment, the compound is an antibody that specifically binds to one or both of Ly6D protein and CD44 protein. The terms “antibody” and “immunoglobulin” are interchangeably used to refer to a glycoprotein or a portion thereof (including single chain antibodies), which is evoked in an animal by an immunogen and which demonstrates specificity to the immunogen, or, more specifically, to one or more epitopes contained in the immunogen. The term “antibody” expressly includes within its scope antigen binding fragments of such antibodies, including, for example, Fab, F(ab′)2, Fd or Fv fragments of an antibody. The antibodies of the invention also include chimeric and humanized antibodies. Antibodies may be polyclonal or monoclonal. The term “polyclonal antibody” refers to an immunoglobulin produced from more than a single clone of plasma cells; in contrast “monoclonal antibody” refers to an immunoglobulin produced from a single clone of plasma cells. The term “specifically binds” refers to the fact that the antibody has higher affinity for the kinase then for other proteins (e.g. serum albumin, and the like) and will therefore display a stronger signal (e.g. in an in vitro assay) over background (e.g. at least 2 to 1, preferably more than 3:1, more preferably at least 5:1, still more preferably 10:1 over background).

Antibodies contemplated to be within the scope of the invention include naturally occurring antibodies as well as non-naturally occurring antibodies, including, for example, single chain antibodies, chimeric, bifunctional and humanized antibodies, as well as antigen-binding fragments thereof. Naturally occurring antibodies may be generated in any species including murine, rat, rabbit, hamster, human, and simian species using methods known in the art. Non-naturally occurring antibodies can be constructed using solid phase peptide synthesis, can be produced recombinantly or can be obtained, for example, by screening combinatorial libraries consisting of variable heavy chains and variable light chains as previously described (Huse et al., Science 246:1275-1281 (1989)). These and other methods of making, for example, chimeric, humanized, CDR-grafted, single chain, and bifunctional antibodies are well known to those skilled in the art (Winter and Harris, Immunol. Today 14:243-246 (1993); Ward et al., Nature 341:544-546 (1989); Hilyard et al., Protein Engineering: A practical approach (IRL Press 1992); and Borrabeck, Antibody Engineering, 2d ed. (Oxford University Press 1995).

As used herein, the term “antibody” when used in reference to an anti-Ly6D antibody and anti-CD44 antibody, refers to an antibody which specifically binds to one or more epitopes on a Ly6D protein (or portion thereof) and a CD44 protein (or portion thereof), respectively. In one embodiment, an anti-Ly6D antibody (or antigen binding fragment thereof) or anti-Ly6D antibody and anti-CD44 antibody (or antigen binding fragment thereof), is characterized by having specific binding activity for Ly6D protein and CD44 protein, respectively, of at least about 1×105M-1, more preferably at least about 1×106M-1, and yet more preferably at least about 1×107M-1.

Those skilled in the art know how to make polyclonal and monoclonal antibodies that are specific to a desirable polypeptide. For example, monoclonal antibodies may be generated by immunizing an animal (e.g., mouse, rabbit, etc.) with a desired antigen and the spleen cells from the immunized animal are immortalized, commonly by fusion with a myeloma cell.

Immunization with antigen may be accomplished in the presence or absence of an adjuvant (e.g., Freund's adjuvant). Typically, for a mouse, 10 μg antigen in 50-200 μl adjuvant or aqueous solution is administered per mouse by subcutaneous, intraperitoneal or intra-muscular routes. Booster immunization may be given at intervals (e.g., 2-8 weeks). The final boost is given approximately 2-4 days prior to fusion and is generally given in aqueous form rather than in adjuvant.

Spleen cells from the immunized animals may be prepared by teasing the spleen through a sterile sieve into culture medium at room temperature, or by gently releasing the spleen cells into medium by pressure between the frosted ends of two sterile glass microscope slides. The cells are harvested by centrifugation (400×g for 5 min.), washed and counted.

Spleen cells are fused with myeloma cells to generate hybridoma cell lines. Several mouse myeloma cell lines which have been selected for sensitivity to hypoxanthine-aminopterin-thymidine (HAT) are commercially available and may be grown in, for example, Dulbecco's modified Eagle's medium (DMEM) (Gibco BRL) containing 10-15% fetal calf serum. Fusion of myeloma cells and spleen cells may be accomplished using polyethylene glycol (PEG) or by electrofusion using protocols that are routine in the art. Fused cells are distributed into 96-well plates followed by selection of fused cells by culture for 1-2 weeks in 0.1 ml DMEM containing 10-15% fetal calf serum and HAT. The supernatants are screened for antibody production using methods well known in the art. Hybridoma clones from wells containing cells that produce antibody are obtained (e.g., by limiting dilution). Cloned hybridoma cells (4-5×106) are implanted intraperitoneally in recipient mice, preferably of a BALB/c genetic background. Sera and ascites fluids are typically collected from mice after 10-14 days.

The invention also contemplates humanized antibodies that are specific for at least a portion of Ly6D protein and/or at least a portion of CD44 protein. Humanized antibodies may be generated using methods known in the art, including those described in U.S. Pat. Nos. 5,545,806; 5,569,825 and 5,625,126, the entire contents of which are incorporated by reference. Such methods include, for example, generation of transgenic non-human animals which contain human immunoglobulin chain genes and which are capable of expressing these genes to produce a repertoire of antibodies of various isotypes encoded by the human immunoglobulin genes.

2. Nucleic Acid Sequences

In an alternative embodiment, compounds useful in the invention's methods include a nucleic acid sequence. The terms “nucleic acid sequence” and “nucleotide sequence” as used herein refer to two or more nucleotides that are covalently linked to each other. Included within this definition are oligonucleotides, polynucleotide, and fragments and/or portions thereof, DNA and/or RNA of genomic and/or synthetic origin which may be single- or double-stranded, and represent the sense or antisense strand. Nucleic acid sequences that are particularly useful in the instant invention include, without limitation, RNA interference sequences, antisense sequences, and ribozymes. The nucleic acid sequences are contemplated to bind to genomic DNA sequences or RNA sequences that encode at least a portion of Ly6D protein and/or at least a portion of CD44 protein, thereby reducing expression of and/or the biological activity of Ly6D protein and/or CD44 protein. RNA interferences sequences, antisense sequences, and ribozyme sequences may be delivered to cells by transfecting the cell with a vector that expresses these sequences as an mRNA molecule. Alternatively, delivery may be accomplished by entrapping the RNA interference sequences, ribozymes and antisense sequences in liposomes.

a. RNA Interference

As used herein, the term “RNA interference” or “RNAi” refers to the silencing or decreasing of gene expression by shRNA, RNAi and/or siRNA. “RNA interference sequence” refers to an shRNA sequence, RNAi sequence and/or siRNA sequence that specifically binds to a target mRNA sequence. RNA interference is the process of sequence-specific, post-transcriptional gene silencing in animals and plants, initiated by shRNA and/or siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial. In both plants and animals, RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger, although the protein components of this activity are unknown. However, the 22-nucleotide RNA sequences are homologous to the target gene that is being suppressed. Thus, the 22-nucleotide sequences appear to serve as guide sequences to instruct a multicomponent nuclease, RISC, to destroy the specific mRNAs. Carthew (2001) has reported (Curr. Opin. Cell Biol. 13(2):244-248; herein incorporated by reference) that eukaryotes silence gene expression in the presence of dsRNA homologous to the silenced gene. Biochemical reactions that recapitulate this phenomenon generate RNA fragments of 21 to 23 nucleotides from the double-stranded RNA. These stably associate with an RNA endonuclease, and probably serve as a discriminator to select mRNAs. Once selected, mRNAs are cleaved at sites 21 to 23 nucleotides apart.

As used herein, the term “shRNA” or “short hairpin RNA” refers to a sequence of ribonucleotides comprising a single-stranded RNA polymer that makes a tight hairpin turn on itself to provide a “double-stranded” or duplexed region. shRNA can be used to silence gene expression via RNA interference. shRNA hairpin is cleaved into short interfering RNAs (siRNA) by the cellular machinery and then bound to the RNA-induced silencing complex (RISC). It is believed that the complex inhibits RNA as a consequence of the complexed siRNA hybridizing to and cleaving RNAs that match the siRNA that is bound thereto.

Methods for designing an shRNA sequence are known in the art including web-based software BLOCK-iTTM RNAi Designer available online at rnaidesigner.Invitrogen.com/rnaiexpress/. The BLOCK-iTTM RNAi Designer software may be used to design siRNA, Stealth RNAi™ siRNA, miR RNAi inserts and shRNA inserts for any target sequence. In one embodiment, targeting sequences may be further prioritized based on guidelines described by Ui-Tei et al. (Ui-Tei et al. Nucleic Acids Res., 32, 936-948). These criteria are exemplified by: i. A/U at the 5′ end of the antisense strand; ii. G/C at the 5′ end of the sense strand; iii. AU-richness in the 5′ terminal one-third of the antisense strand; and iv. the absence of any GC stretch over 9 bp in length.

The term “siRNA” refers to short interfering RNA. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The terms “hpRNA” and “hairpin RNA” refer to self-complementary RNA that forms hairpin loops and functions to silence genes (e.g. Wesley et al. (2001) The Plant Journal 27(6):581-590; herein incorporated by reference). The term “ihpRNA” refers to intron-spliced hpRNA that functions to silence genes.

b. Antisense Sequences

Antisense sequences have been successfully used to inhibit the expression of several genes (Markus-Sekura (1988) Anal. Biochem. 172:289-295; Hambor et al. (1988) J. Exp. Med. 168:1237-1245; and patent EP 140 308), including the gene encoding VCAM1, one of the integrin α4β1 ligands (U.S. Pat. No. 6,252,043, incorporated in its entirety by reference). The terms “antisense DNA sequence” and “antisense sequence” as used herein interchangeably refer to a deoxyribonucleotide sequence whose sequence of deoxyribonucleotide residues is in reverse 5′ to 3′ orientation in relation to the sequence of deoxyribonucleotide residues in a sense strand of a DNA duplex. A “sense strand” of a DNA duplex refers to a strand in a DNA duplex that is transcribed by a cell in its natural state into a “sense mRNA.” Sense mRNA generally is ultimately translated into a polypeptide. Thus, an “antisense DNA sequence” is a sequence which has the same sequence as the non-coding strand in a DNA duplex, and which encodes an “antisense RNA” (i.e., a ribonucleotide sequence whose sequence is complementary to a “sense mRNA” sequence). The designation (−) (i.e., “negative”) is sometimes used in reference to the antisense strand, with the designation (+) sometimes used in reference to the sense (i.e., “positive”) strand. Antisense RNA may be produced by any method, including synthesis by splicing an antisense DNA sequence to a promoter that permits the synthesis of antisense RNA. The transcribed antisense RNA strand combines with natural mRNA produced by the cell to form duplexes. These duplexes then block either the further transcription of the mRNA or its translation, or promote its degradation.

Any antisense sequence is contemplated to be within the scope of this invention if it is capable of reducing the level of expression of the invention's sequences to a quantity which is less than the quantity of sequence expression in a control tissue which is (a) not treated with the antisense sequence, (b) treated with a sense sequence, or (c) treated with a nonsense sequence.

Antisense Ly6D sequences and antisense CD44 sequences include, for example, sequences which are capable of hybridizing with at least a portion of Ly6D cDNA and CD44 cDNA, respectively, under high stringency or medium stringency conditions. Antisense sequences may be designed using approaches known in the art. In a preferred embodiment, the antisense Ly6D sequences and antisense CD44 sequences are designed to be hybridizable to Ly6D mRNA and to CD44 mRNA, respectively, that is encoded by the coding region of the Ly6D gene and CD44 gene, respectively. Alternatively, antisense Ly6D sequences and antisense CD44 sequences may be designed to reduce transcription by hybridizing to upstream nontranslated sequences, thereby preventing promoter binding to transcription factors.

In a preferred embodiment, the antisense oligonucleotide sequences of the invention range in size from about 8 to about 100 nucleotide residues. In yet a more preferred embodiment, the oligonucleotide sequences range in size from about 8 to about 30 nucleotide residues. In a most preferred embodiment, the antisense sequences have 20 nucleotide residues.

The antisense oligonucleotide sequences that are useful in the methods of the instant invention may comprise naturally occurring nucleotide residues as well as nucleotide analogs. Nucleotide analogs may include, for example, nucleotide residues that contain altered sugar moieties, altered inter-sugar linkages (e.g., substitution of the phosphodiester bonds of the oligonucleotide with sulfur-containing bonds, phosphorothioate bonds, alkyl phosphorothioate bonds, N-alkyl phosphoramidates, phosphorodithioates, alkyl phosphonates and short chain alkyl or cycloalkyl structures), or altered base units. Oligonucleotide analogs are desirable, for example, to increase the stability of the antisense oligonucleotide compositions under biologic conditions since natural phosphodiester bonds are not resistant to nuclease hydrolysis. Oligonucleotide analogs may also be desirable to improve incorporation efficiency of the oligonucleotides into liposomes, to enhance the ability of the compositions to penetrate into the cells where the nucleic acid sequence whose activity is to be modulated is located, in order to reduce the amount of antisense oligonucleotide needed for a therapeutic effect thereby also reducing the cost and possible side effects of treatment.

Antisense oligonucleotide sequences may be synthesized using any of a number of methods known in the art, as well as using commercially available services (e.g., Genta, Inc.). Synthesis of antisense oligonucleotides may be performed, for example, using a solid support and commercially available DNA synthesizers. Alternatively, antisense oligonucleotides may also be synthesized using standard phosphoramidate chemistry techniques. For example, it is known in the art that for the generation of phosphodiester linkages, the oxidation is mediated via iodine, while for the synthesis of phosphorothioates, the oxidation is mediated with 3H-1,2-benzodithiole-3-one,1,-dioxide in acetonitrile for the step-wise thioation of the phosphite linkages. The thioation step is followed by a capping step, cleavage from the solid support, and purification on HPLC, e.g., on a PRP-1 column and gradient of acetonitrile in triethylammonium acetate, pH 7.0.

In one embodiment, the antisense DNA sequence is a “Ly6D antisense DNA sequence” (i.e., an antisense DNA sequence which is designed to bind with at least a portion of the Ly6D genomic sequence or with Ly6D mRNA). In another embodiment, the antisense DNA sequence is a “CD44 antisense DNA sequence” (i.e., an antisense DNA sequence which is designed to bind with at least a portion of the CD44 genomic sequence or with CD44mRNA).

c. Ribozyme

In some alternative embodiments, compounds useful in the invention's methods include a ribozyme. Ribozyme sequences have been successfully used to inhibit the expression of several genes including the gene encoding VCAM1, which is one of the integrin α4β1 ligands (U.S. Pat. No. 6,252,043, incorporated in its entirety by reference).

The term “ribozyme” refers to an RNA sequence that hybridizes to a complementary sequence in a substrate RNA and cleaves the substrate RNA in a sequence specific manner at a substrate cleavage site. Typically, a ribozyme contains a “catalytic region” flanked by two “binding regions.” The ribozyme binding regions hybridize to the substrate RNA, while the catalytic region cleaves the substrate RNA at a “substrate cleavage site” to yield a “cleaved RNA product.” The nucleotide sequence of the ribozyme binding regions may be completely complementary or partially complementary to the substrate RNA sequence with which the ribozyme binding regions hybridize. Complete complementarity is preferred, in order to increase the specificity, as well as the turnover rate (i.e., the rate of release of the ribozyme from the cleaved RNA product), of the ribozyme. Partial complementarity, while less preferred, may be used to design a ribozyme binding region containing more than about 10 nucleotides. While contemplated to be within the scope of the claimed invention, partial complementarity is generally less preferred than complete complementarity since a binding region having partial complementarity to a substrate RNA exhibits reduced specificity and turnover rate of the ribozyme when compared to the specificity and turnover rate of a ribozyme which contains a binding region having complete complementarity to the substrate RNA. A ribozyme may hybridize to a partially or completely complementary DNA sequence but cannot cleave the hybridized DNA sequence since ribozyme cleavage requires a 2′-OH on the target molecule, which is not available on DNA sequences.

The ability of a ribozyme to cleave at a substrate cleavage site may readily be determined using methods known in the art. These methods include, but are not limited to, the detection (e.g., by Northern blot analysis as described herein, reverse-transcription polymerase chain reaction (RT-PCR), in situ hybridization and the like) of reduced in vitro or in vivo levels of RNA which contains a ribozyme substrate cleavage site for which the ribozyme is specific, compared to the level of RNA in controls (e.g., in the absence of ribozyme, or in the presence of a ribozyme sequence which contains a mutation in one or both unpaired nucleotide sequences which renders the ribozyme incapable of cleaving a substrate RNA).

Ribozymes contemplated to be within the scope of this invention include, but are not restricted to, hammerhead ribozymes (See e.g., Reddy et al., U.S. Pat. No. 5,246,921; Taira et al., U.S. Pat. No. 5,500,357, Goldberg et al., U.S. Pat. No. 5,225,347, the contents of each of which are herein incorporated by reference), Group I intron ribozyme (Kruger et al. (1982) Cell 31: 147-157), ribonuclease P (Guerrier-Takada et al. (1983) Cell 35: 849-857), hairpin ribozyme (Hampel et al., U.S. Pat. No. 5,527,895 incorporated by reference), and hepatitis delta virus ribozyme (Wu et al. (1989) Science 243:652-655).

A ribozyme may be designed to cleave at a substrate cleavage site in any substrate RNA so long as the substrate RNA contains one or more substrate cleavage sequences, and the sequences flanking the substrate cleavage site are known. In effect, expression in vivo of such ribozymes and the resulting cleavage of RNA transcripts of a gene of interest reduces or ablates expression of the corresponding gene.

For example, where the ribozyme is a hammerhead ribozyme, the basic principle of a hammerhead ribozyme design involves selection of a region in the substrate RNA which contains a substrate cleavage sequence, creation of two stretches of antisense oligonucleotides (i.e., the binding regions) which hybridize to sequences flanking the substrate cleavage sequence, and placing a sequence which forms a hammerhead catalytic region between the two binding regions.

In order to select a region in the substrate RNA which contains candidate substrate cleavage sites, the sequence of the substrate RNA needs to be determined. The sequence of RNA encoded by a genomic sequence of interest is readily determined using methods known in the art. For example, the sequence of an RNA transcript may be arrived at either manually, or using available computer programs (e.g., GENEWORKS, from IntelliGenetic Inc., or RNADRAW available from the internet at ole@mango.mef.ki.se), by changing the T in the DNA sequence encoding the RNA transcript to a U.

Substrate cleavage sequences in the target RNA may be located by searching the RNA sequence using available computer programs. For example, where the ribozyme is a hammerhead ribozyme, it is known in the art that the catalytic region of the hammerhead ribozyme cleaves only at a substrate cleavage site which contains a NUH, where N is any nucleotide, U is a uridine, and H is a cytosine (C), uridine (U), or adenine (A) but not a guanine (G). The U-H doublet in the NUH cleavage site does not include a U-G doublet since a G would pair with the adjacent C in the ribozyme and prevent ribozyme cleavage. Typically, N is a G and H is a C. Consequently, GUC has been found to be the most efficient substrate cleavage site for hammerhead ribozymes, although ribozyme cleavage at CUC is also efficient.

In a preferred embodiment, the substrate cleavage sequence is located in a loop structure or in an unpaired region of the substrate RNA. Computer programs for the prediction of RNA secondary structure formation are known in the art and include, for example, “RNADRAW”, “RNAFOLD” (Hofacker et al. (1994) Monatshefte F. Chemie 125:167-188; McCaskill (1990) Biopolymers 29:1105-1119). “DNASIS” (Hitachi), and ATHE VIENNA PACKAGE.

In addition to the desirability of selecting substrate cleavage sequences which are located in a loop structure or an unpaired region of the substrate RNA, it is also desirable, though not required, that the substrate cleavage sequence be located downstream (i.e., at the 3′-end) of the translation start codon (AUG or GUG) such that the translated truncated polypeptide is not biologically functional.

In a preferred embodiment, the ribozyme is a “Ly6D ribozyme” (i.e., a ribozyme whose substrate cleavage sequence is designed to hybridize with a portion of Ly6D. In another preferred embodiment, the ribozyme is a “CD44 ribozyme” (i.e., a ribozyme whose substrate cleavage sequence is designed to hybridize with a portion of CD44.

One of skill in the art appreciates that it is not necessary that the two binding regions that flank the ribozyme catalytic region be of equal length. Binding regions that contain any number of nucleotides are contemplated to be within the scope of this invention so long as the desirable specificity of the ribozyme for the RNA substrate and the desirable cleavage rate of the RNA substrate are achieved. One of skill in the art knows that binding regions of longer nucleotide sequence, while increasing the specificity for a particular substrate RNA sequence, may reduce the ability of the ribozyme to dissociate from the substrate RNA following cleavage to bind with another substrate RNA molecule, thus reducing the rate of cleavage. On the other hand, though binding regions with shorter nucleotide sequences may have a higher rate of dissociation and cleavage, specificity for a substrate cleavage site may be compromised.

It is well within the skill of the art to determine an optimal length for the binding regions of a ribozyme such that a desirable specificity and rate of cleavage are achieved. Both the specificity of a ribozyme for a substrate RNA and the rate of cleavage of a substrate RNA by a ribozyme may be determined by, for example, kinetic studies in combination with Northern blot analysis or nuclease protection assays.

In a preferred embodiment, the complementarity between the ribozyme binding regions and the substrate RNA is complete. However, the invention is not limited to ribozyme sequences in which the binding regions show complete complementarity with the substrate RNA. Complementarity may be partial, so long as the desired specificity of the ribozyme for a substrate cleavage site and the rate of cleavage of the substrate RNA are achieved. Thus, base changes may be made in one or both of the ribozyme binding regions as long as substantial base pairing with the substrate RNA in the regions flanking the substrate cleavage sequence is maintained and base pairing with the substrate cleavage sequence is minimized. The term “substantial base pairing” means that greater than about 65%, more preferably greater than about 75%, and yet more preferably greater than about 90% of the bases of the hybridized sequences are base-paired.

It may be desirable to increase the intracellular stability of ribozymes expressed by an expression vector. This is achieved by designing the expressed ribozyme such that it contains a secondary structure (e.g., stem-loop structures) within the ribozyme molecule. Secondary structures which are suitable for stabilizing ribozymes include, but are not limited to, stem-loop structures formed by intra-strand base pairs. An alternative to the use of a stem-loop structure to protect ribozymes against ribonuclease degradation is by the insertion of a stem loop at each end of the ribozyme sequence (Sioud and Drlica (1991) Proc. Natl. Acad. Sci. USA 88:7303-7307). Other secondary structures which are useful in reducing the susceptibility of a ribozyme to ribonuclease degradation include hairpin, bulge loop, interior loop, multibranched loop, and pseudoknot structure as described in “Molecular and Cellular Biology,” Stephen L. Wolfe (Ed.), Wadsworth Publishing Company (1993) p. 575. Additionally, circularization of the ribozyme molecule protects against ribonuclease degradation since exonuclease degradation is initiated at either the 5=-end or 3=-end of the RNA. Methods of expressing a circularized RNA are known in the art (see, e.g., Puttaraju et al. (1993) Nucl. Acids Res. 21:4253-4258).

Once a ribozyme with desirable binding regions, a catalytic region and nuclease stability has been designed, the ribozyme may be produced by any known means including chemical synthesis. Chemically synthesized ribozymes may be introduced into a cell by, for example, microinjection electroporation, lipofection, etc. In a preferred embodiment, ribozymes are produced by expression from an expression vector that contains a gene encoding the designed ribozyme sequence.

### J. Administering Compounds

Agents that are useful in the invention's methods be administered to a subject by various routes including, for example, orally, intranasally, or parenterally, including intravenously, intramuscularly, subcutaneously, intraorbitally, intracapsularly, intrasynovially, intraperitoneally, intracisternally or by passive or facilitated absorption through the skin using, for example, a skin patch or transdermal iontophoresis. Furthermore, the agent can be administered by injection, intubation, via a suppository, orally or topically, the latter of which can be passive, for example, by direct application of an ointment or powder containing the agent, or active, for example, using a nasal spray or inhalant. The agent can also be administered as a topical spray, if desired, in which case one component of the composition is an appropriate propellant. The pharmaceutical composition also can be incorporated, if desired, into liposomes, microspheres or other polymer matrices (Gregoriadis, “Liposome Technology,” Vol. 1, CRC Press, Boca Raton, Fla. 1984). Liposomes, for example, which consist of phospholipids or other lipids, are nontoxic, physiologically acceptable and metabolizable carriers that are relatively simple to make and administer. Liposomes are lipid-containing vesicles having a lipid bilayer as well as other lipid carrier particles that can entrap chemical agents. Liposomes may be made of one or more phospholipids, optionally including other materials such as sterols. Suitable phospholipids include phosphatidyl cholines, phosphatidyl serines, and many others that are well known in the art. Liposomes can be unilamellar, multilamellar or have an undefined lamellar structure. For example, in an individual suffering from a metastatic carcinoma, the agent in a pharmaceutical composition can be administered intravenously, orally or by another method that distributes the agent systemically.

### K. IKK/NF-κB Signaling Pathway in HCC Development

NF-κB, a collection of dimeric transcription factors, first identified based on their interaction with the immunoglobulin light-chain enhancer in B cells¹³, are present in all cells¹⁴. Seven distinct NF-κB proteins can form a variety of dimers, not all of which are active. These proteins include: NF-κB1 (p105 and p50), NF-κB2 (p100 and p52), RelA (p65), RelB, and c-Rel. In non-stimulated cells, most NF-κB dimers are retained in the cytoplasm by binding to inhibitory IκB proteins, except for the dimers formed by p105 and p100, which are inactive and contain intrinsic IκB-like-moieties. In response to proinflammatory stimuli, such as tumor necrosis factor (TNF) or interleukin 1β (IL-1β), the IκB kinase (IKK) complex, composed on the IKKα and IKKβ catalytic subunits and the IKKγ regulatory subunit is activated, resulting in IκB phosphorylation and eventual ubiquitin-mediated degradation, leading to the nuclear entry of freed NF-κB dimers¹⁵. Of the two catalytic subunits, IKKβ is the one which is most critical for IκB degradation, forming the core of what is known as the classical NE-κB activation pathway. By contrast, IKKα is required for the inducible processing of the inactive p100 protein to its active derivative p52, thus forming the core of the so called alternative NF-κB pathway^{15, 16}.

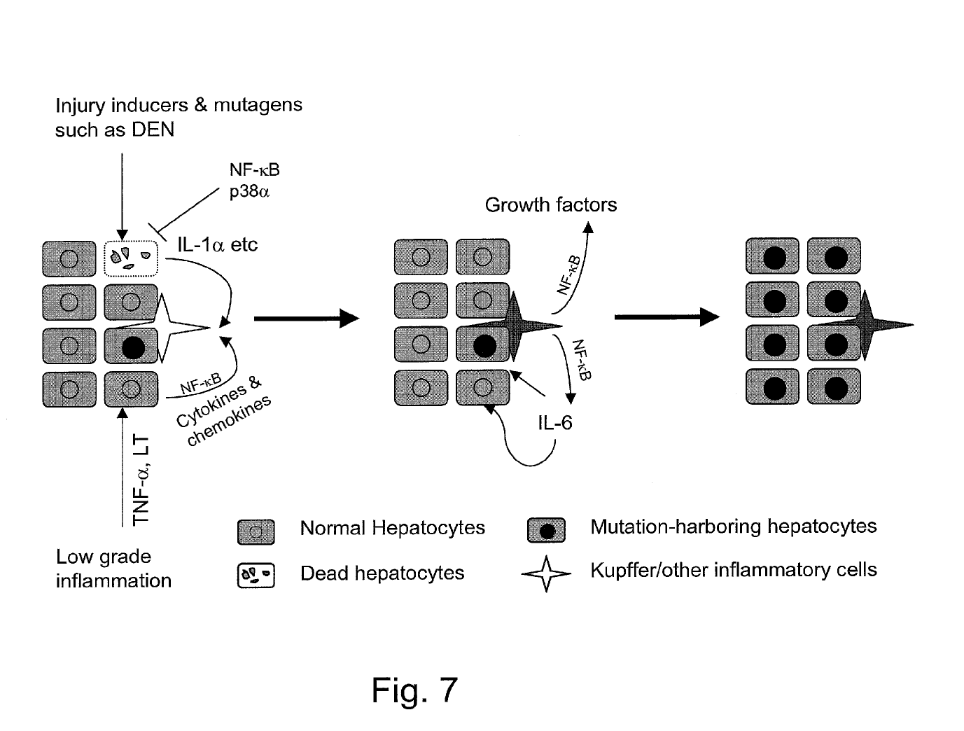

A link between NF-κB and cancer first became evident with the cloning of RelA and the realization of its close kinship with the viral oncoprotein v-Rel¹⁷. The view was further supported by observations of activated NF-κB in many human cancers¹⁸. In addition, the Bcl-3 oncogene, activated by chromosomal translocation in B-cell chronic lymphocytic leukemia, was identified as a member of the IκB family^{19, 20}. More recently, mutations in upstream components of the IKK-NF-κB signaling system were identified in multiple myeloma and are thought to lead to cell autonomous activation of NF-κB, thereby enhancing cell survival and proliferation^{21,22}. FIG. 7 shows the roles of NF-κB signaling in hepatocarcinogenesis. However, extensive search failed to identify NF-κB-activating mutations in most other cancers and most likely cancer-associated constitutive NF-κB activities are the result of exposure to pro-inflammatory stimuli in the tumor microenvironment.

1. Hepatocyte IKK-Dependent NF-κB Signaling Suppresses Liver Cancer Development by Promoting Hepatocyte Survival.

A key role of NF-κB in liver homeostasis was first revealed by studying RelA/p65 deficient mice, which suffer embryonic lethality with extensive liver apoptosis and degeneration²³. This liver apoptosis is induced by TNF and backcrossing of p65 KO mice with TNF- or TNF receptor 1 (TNFR1)-deficient mice prevents liver damage and the lethal phenotype²³⁻²⁵. Later on, IKKβ^{26, 27} and IKKγ^{28, 29} knockout mice were found to exhibit very similar phenotypes. These genetic studies clearly demonstrate an anti-apoptotic role for IKK-dependent NF-κB signaling in hepatocytes, mainly during early liver development.

The role of IKK-dependent classical NF-κB signaling in adult mouse liver physiology, however, is more complex. Mice with hepatocytes-specific ablation of IKKβ (Alb-Cre/Ikkβ^{F/F} or Ikkβ^{Δhep} mice) develop normally and their livers are not even sensitive to administration of LPS, a strong TNF inducer^{30, 31}. However, careful analysis suggests that residual hepatocyte IKK activity (presumably from IKKα) in Ikkβ^{Δhep} mice may be sufficient for TNF-induced NF-κB activation, which is completely blocked by an additional ablation of IKKα (Ikkα/Ikkβ^{Δhep} mice)³⁰. These results suggest that IKKα and IKKβ in adult hepatocytes may have somewhat redundant functions in suppressing apoptosis and necrosis. Indeed, unlike Ikkβ^{Δhep} mice, Ikkα/Ikkβ^{Δhep} mice or mice deficient of regulatory component IKKγ in hepatocytes (Ikkγ^{Δhep} mice) suffer from extensive hepatocyte death and liver failure upon TNF-inducing challenges^{30, 32}. Furthermore, Ikkγ^{Δhep} or Ikkα/Ikkβ^{Δhep} mice exhibit spontaneous liver damage, which is not seen in Ikkβ^{Δhep} mice³¹⁻³³. Like Ikkβ^{Δhep} mice, mice deleted of RelA in hepatocytes (RelA^{Δhep} mice) are also healthy unless challenged and exposed to TNF. Based on available evidence, it is safe to conclude that IKK/NF-κB pathway is important for hepatocyte survival and maintenance of liver homeostasis in response to various environmental challenges that can induce the production of TNF and other hepatotoxic cytokines.

The activated IKK/NF-κB pathway may play a tumor-promoting role by protecting tumor cells from death or enhancing their proliferation. This hypothesis was first tested in a mouse model of azoxymethane (AOM)+dextrane sulfate sodium (DSS)-induced colitis-associated cancer (CAC). Conditional disruption of the Ikkβ gene in intestinal epithelial cells (IEC) greatly reduced the development of colonic adenomas and resulted in increased apoptotic elimination of AOM-induced premalignant cells³⁴. However, strikingly different results were obtained in the diethylnitrosamine (DEN)-induced mouse HCC model. DEN is a pro-carcinogen that, upon metabolic activation in zone 3 hepatocytes, forms bulky DNA adducts³⁵. Upon subsequent cell proliferation, some of these DNA adducts are fixed into permanent genetic alterations that may cause activation of oncogenes, such as β-catenin (He and Karin, unpublished results). A single dose of DEN given to two-weeks-old mice is sufficient to induce HCC in 100% of male mice. However, when DEN is given to male mice that are older than 4 weeks of age, it is no longer effective in HCC induction on its own and requires assistance from tumor promoters, such as phenobarbitol. This age-dependent difference in carcinogenic efficacy is not likely to be due to altered metabolic activation of DEN^{36, 37}. The main reason that DEN is not a complete carcinogen in mice that are more than 4 weeks old is the nearly complete absence of proliferating hepatocytes³⁸. Thus, any agent that induces hepatocyte proliferation should function as a tumor promoter. Indeed, partial hepatectomy after DEN administration results in effective hepatocarcinogenesis in older mice³⁸. It was found that liver specific disruption of IKKβ greatly enhances DEN-induced hepatocyte death relative to wild type mice³⁹. Although this may enhance the elimination of DEN-damaged hepatocytes, it should be noted that enhanced hepatocyte death also results in enhanced compensatory proliferation. Consequently, Ikkβ^{Δhep} mice are 3-4 fold more susceptible to DEN-induced HCC development than wild type mice³⁹. An even more striking effect on HCC development is seen upon the conditional deletion of hepatocyte IKKγ/NEMO³³. In this case, Ikkγ^{Δhep} mice exhibit spontaneous liver damage and sequentially develop hepatosteatosis, hepatitis, liver fibrosis, and HCC without any known exposure to a carcinogen³³.

Multiple mechanisms were proposed to explain the pro-survival function of IKK/NF-κB pathway, which can either enhance tumor development (as it does in the colon) or attenuate tumor development (as it does in the liver)⁴⁰. In the liver a critical pro-survival mechanism involves NF-κB's ability to maintain anti-oxidant defenses by controlling the expression of several key reactive oxygen species (ROS)-scavenging proteins^{41, 42}. Mice that lack IKKβ exhibit extensive ROS accumulation in their livers shortly after injection of DEN, whose metabolism in zone 3 hepatocytes results in ROS production³⁹. Increased ROS accumulation is also seen in livers of unchallenged Ikkγ^{Δhep} mice³³. ROS accumulation in the liver can be prevented by dietary administration of the potent anti-oxidant butylated hydroxyanisole (BHA). Indeed, liver damage, compensatory proliferation and hepatocarcinogenesis in both Ikkβ^{Δhep} and Ikkγ^{Δhep} mutant mice are reversed by BHA consumption^{33, 39}. Excessive ROS accumulation promotes cell death through various mechanisms, including prolonged JNK activation⁴¹. In support of this view, increased JNK phosphorylation and kinase activity are observed in livers of Ikkγ^{Δhep} mice and DEN-challenged Ikkβ^{Δhep} mice^{33, 39}. Importantly, reduced hepatocyte death, less compensatory proliferation and suppressed hepatocarcinogenesis were observed upon crossing of Ikkβ^{Δhep} mice to Jnk1^{-/-} mice⁴³. Therefore, the IKK/NF-κB pathway maintains hepatocyte survival by preventing ROS accumulation and excessive JNK activation, thereby reducing liver damage, proliferation and cancer development.

2. Hepatocyte IKK/NF-κB Promotes HCC Development by Maintaining Liver Inflammatory Responses.

In sharp contrast to the tumor-suppressing role of hepatocyte IKK/NF-κB signaling in the mouse models described above, in other HCC models the NF-κB pathway was found to promote tumor development. The first example came from the elegant work of Pikarsky and colleagues⁴⁴. They employed Mdr2^{-/-} mice, which spontaneously develop cholangitis due to defective cholesterol phospholipid secretion in the bile⁴⁵. These mice developed low-grade chronic liver inflammation that eventually results in the development of HCC. It was found that NF-κB was activated in Mdr2^{-/-} hepatocytes, although the initial stimulus leading to NF-κB activation has not been fully identified. NF-κB activation promotes low amounts of TNF production and paracrine TNF signaling maintain NF-κB activation in Mdr2^{-/-} hepatocytes. Correspondingly, treatment of Mdr2^{-/-} mice with a neutralizing TNF antibody inhibits NF-κB activation in hepatocytes and decreases expression of NF-κB-dependent anti-apoptotic genes⁴⁴. The authors examined the tumorigenic function of hepatocyte NF-κB by expressing a nondegradable form of IκBα from a doxycycline-regulated liver-specific promoter and found that inhibition of NF-κB activation retarded and reduced HCC development in Mdr2^{-/-} mice⁴⁴. A similar tumor-promoting role for hepatocyte NF-κB was observed in transgenic mice that express lymphotoxin (LT)α:β heterotrimers in hepatocytes⁴⁶. LTα:β transgenic mice develop liver inflammation, evidenced by chronic penetration of T, B and dendritic cells into their livers and elevated production of cytokines such as IL-1β, IFNγ and IL-6⁴⁶. Chronic liver inflammation is accompanied by increased hepatocyte proliferation that eventually leads to appearance of HCC in old mice. Crossing of LTα:β transgenic mice with Ikkβ^{Δhep} mice prevented liver inflammation and reduced HCC development, suggesting that in this case IKKβ activation in hepatocytes is tumor promoting because it is required to sustain the chronic inflammatory response initiated by LTα:β expression⁴⁸.

Notably in both Mdr2^{-/-} and LTαβ-transgenic mice, HCC development depends on chronic low grade inflammation and no liver injury has been observed either prior to or subsequent to NF-κB inhibition^{44, 46}. Thus in these models, in contrast to the injury-driven Ikkβ^{Δhep}+DEN and Ikkγ^{Δhep} models, the main function of NF-κB in hepatocytes appears to be the production of cytokines that maintain the inflammatory microenvironment in which these tumors develop.

3. IKK/NF-κB in Liver Myeloid Cells Promotes Liver Cancer Development Through IL-6 and Liver Inflammatory Responses.

Different environmental challenges and stimuli are sensed by resident myeloid cells (Kupffer cells in liver), which initiate an inflammatory response aimed to remove the insults and repair the injured tissue. Activated Kupffer cells produce a panel of inflammatory cytokines and growth factors in an IKK/NF-κB-dependent manner. In the DEN model, where hepatocyte IKK/NF-κB signaling was found to inhibit HCC development, activation of IKKβ/NF-κB in Kupffer cells promotes tumor development³⁹. Deletion of IKKβ in liver myeloid cells in addition to hepatocytes diminished the production of pro-inflammatory cytokines, such as IL-6 and TNF, reduced liver compensatory proliferation and strongly inhibited DEN-induced HCC development³⁹. Deletion of IKKβ in Kupffer cells was also found to inhibit the metastatic growth of Lewis lung carcinoma cells in liver⁴⁷. The mechanism by which DEN administration leads to IKK/NF-κB activation in Kupffer cells was found to depend on the release of IL-1α by necrotic hepatocytes which activates an MyD88-dependent signaling pathway upon binding to IL-1 receptor (IL-1R) on Kupffer cells. Inhibition of IL-1R signaling or ablation of MyD88 were found to attenuate DEN-induced HCC development⁴⁸.

One of the most important NF-κB-dependent cytokines that is produced by activated Kupffer cells is IL-6. Interestingly, DEN-treated female mice which unlike male mice are resistant to DEN-induced HCC development, produce less IL-6 than similarly treated male mice⁴⁹. IL-6 is a major STAT3 activator in liver and male mice lacking IL-6 exhibit reduced DEN-induced STAT3 activation and are as protected from HCC development as wild type female⁴⁹. These results suggest that the striking male preference in HCC development in both human and mice may be due to differential IL-6 production^{8, 49}. Whereas IL-6 ablation abolishes the male bias in DEN-induced HCC development, ovariectomy enhances IL-6 production and augments HCC induction in female mice⁴⁹. It is likely that gender-specific differences in IL-6 expression also affect the incidence of human HCC, as serum IL-6 is higher after menopause^{50, 51} and postmenopausal women display higher HCC incidence than premenopausal women⁸. Moreover, expression of IL-6 is elevated in both liver cirrhosis and HCC^{52, 53} and was recently found to correlate with rapid progression from viral hepatitis to HCC^{54, 55}. Precise mechanisms by which elevated IL-6 promotes HCC development are not known, but some of IL-6 functions are likely mediated by activation of STAT3.

### L. STAT3 in Liver Cancer

1. STAT3 Signaling is Turned on in Human HCC

STAT3 was first identified and cloned from mouse liver cDNA library in a study of IL-6 signaling^{56, 57}. STAT3 belongs to the signal transducer and activator of transcription (STAT) family. Like its relatives, STAT3 is inactive in non-stimulated cells, but is rapidly activated by various cytokines and growth factors, such as IL-6 and EGF family members, as well as hepatocyte growth factor (HGF)^{58, 59}. STAT3 activation requires phosphorylation of a critical tyrosine residue (Tyr705), which mediates its dimerization that is a pre-requisite for nucleus entry and DNA binding⁶⁰. The phosphorylation of STAT3 at Tyr705 is most commonly mediated by Janus kinases (JAKs), especially JAK2, but its activity is also subject to fine tuning by other mechanisms, including serine (Ser727) phosphorylation⁶¹ and reversible acetylation⁶². Activation of STAT3 also turns on strong negative feedback loops involving SHP phosphatases and suppressor of cytokine signaling 3 (SOCS3)⁶³. These feedback mechanisms dampen STAT3 activity and ensure that cytokine-induced STAT3 activation is a transient event in normal cells. However, in cancer cells STAT3 is often found to be constitutively activated⁶⁴.

We have examined a large number of human HCC specimens and detected phosphorylated (i.e. activated) STAT3 in approximately 60% of them, with STAT3-positive tumors being more aggressive⁶⁵. These findings are consistent with those of other studies in which STAT3 was found to be activated in the majority of HCCs with poor prognosis and not in surrounding non-tumor tissue or in normal liver⁶⁶. However, the events that lead to STAT3 activation in human HCC are not known. Interestingly, activating mutations in the gene encoding the gp130 signaling subunit of IL-6 receptor family members were identified in benign hepatic adenomas⁶⁷. When combined with a β catenin activating mutation, these mutations, which cause STAT3 activation, lead to HCC development⁶⁷. Nevertheless, STAT3-activating mutations are rare in human cancers. Most likely, as discussed above for NF-κB, STAT3 in cancer cells is activated by cytokines and growth factors that are produced within the tumor microenvironment. Indeed, the expression of IL-6, one of the major STAT3 activating cytokines, is elevated in human liver diseases and HCC^{52, 53}. In addition, many HCC risk factors, including HCV infection and hepatosteatosis, cause oxidative stress⁶⁸⁻⁷⁰ and just like JNK STAT3 can also be activated in response to ROS accumulation⁶⁵. As discussed herein, NF-κB-induced expression of anti-oxidants prevents inadvertent activation of STAT3 by ROS accumulation, but it needs to be determined whether NF-κB activity is down-regulated during human hepatocarcinogenesis to allow STAT3 activation. Nevertheless, the majority of STAT3-positive HCCs do not exhibit NF-κB activation and most NF-κB positive HCCs do not show activated STAT3⁶⁵. However, the main cause of STAT3 activation in human HCC could simply be the elevated expression of IL-6 and related cytokines, such as IL-11 and IL-22.

2. STAT3 Promotes HCC Development in Mouse Models.

Germ line ablation of Stat3 results in early embryonic lethality⁷¹. In fact, loss of STAT3 is lethal even to embryonic stem cells^{72, 73}, underscoring a critical role for STAT3 in cell growth and/or survival. To overcome these problems, a number of tissue specific Stat3 knockout mouse strains were generated to allow STAT3 deletion in differentiated cells⁷⁴. Using such conditional STAT3 knockout mice, it has been shown that STAT3 is required for tumorigenesis in mouse skin⁷⁵, intestine^{76, 77}, and liver⁶⁵. Such results left little doubt that STAT3 is a critical oncogenic transcription factor and an attractive target for cancer therapy⁷⁸.

We used hepatocyte-specific STAT3 deficient mice Stat3^{Δhep} to examine the role of STAT3 in DEN-induced liver tumorigenesis. Stat3^{Δhep} mice were found to exhibit more than a 6-fold reduction in HCC load relative to Stat3^{F/F} mice⁶⁵. Furthermore, tumors in Stat3^{Δhep} mice were smaller, suggesting that STAT3 may play a role in HCC cell proliferation and/or survival. We derived cell lines from DEN-induced HCCs (dih cells) of Stat3^{F/F} mice. Deletion of STAT3 in cultured Stat3^{F/F} dih cells, accomplished by infecting the cells with a Cre-expressing adenovirus, resulted in cell death, suggesting that activated STAT3 is required for the survival of HCC cells. Although dih cells that are completely STAT3-deficient cannot survive, cells with a partial reduction of STAT3 expression, accomplished by shRNA transduction are viable, but exhibit a senescent phenotype and fail to form subcutaneous tumors upon transplantation⁶⁵.

Interestingly, dependence on STAT3 for survival is also seen in anaplastic large cell lymphomas that spontaneously appear in NPM-ALK transgenic mice, which invariably show STAT3 activation⁷⁹. The lymphoma cells rapidly die when depleted of STAT3 in vitro⁷⁹. Given this strict dependence on STAT3 for survival, it is puzzling to find that a few tumors can still develop in the complete absence of STAT3 in both the DEN-induced HCC model and in NPM-ALK transgenic mice^{65, 79}. It is plausible that an alternative pathway can be activated in STAT3-null tumors but this pathway is hardly active in the presence of STAT3⁷⁸.

3. STAT3 as a Therapeutic Target in Human HCC

As compelling data continue to accumulate STAT3 has become an attractive molecule target for the treatment and prevention of human malignancies. While safety is a primary concern, given the embryonic lethality of STAT3-null mice, with the use of STAT3 inhibitors, tissue-specific Stat3 ablation experiments indicate that STAT3 is not required for the survival of differentiated cells. In addition to the Cre-mediated ablation of floxed Stat3 alleles, it has been possible to achieve more than 95% reduction in STAT3 expression in mouse liver by systemic administration of Stat3 anti-sense oligonucleotides for up to 2 months and the mice tolerated this treatment quite well (G. He and Y. Kim, unpublished results). These results provide supportive evidence that it may be safe to target STAT3 for human cancer therapy.

Different types of STAT3 inhibitors were designed to either directly target STAT3 by inhibiting its dimerization, DNA binding, or nuclear entry or through the targeting of upstream components in the STAT3 activation pathway^{80, 81}. S3I-201 is a direct STAT3 inhibitor that blocks both STAT3 dimerization and DNA-binding and transcriptional activities⁸². Treatment of tumor xenografts derived from a human breast cancer cell line with constitutive STAT3 activity with S3I-201 resulted in inhibition of tumor growth⁸². The therapeutic effect of S3I-201 on xenografts of the human HCC cell line Huh-7 was also examined and it was found that at a dose of 5 mg/kg given every other day, S3I-201 inhibited STAT3 tyrosine phosphorylation and tumor growth⁸³. Another widely used STAT3 inhibitor is AG490 which blocks activation of STAT3 by inhibiting the upstream kinase JAK2⁸⁴. We have tested the effect of S3I-201 and AG490 on the in vivo tumorigenic growth of dih cells and found effective inhibition of STAT3 activity and tumor growth⁶⁵. The higher is the level of STAT3 in a tumor cell line, the more susceptible it is to STAT3 inhibition⁶⁵.

Despite the testing of a number of different STAT3 inhibitors, their overall anti-tumor effects have not been overly impressive⁸⁰. One explanation is that most of currently available STAT3 inhibitors target the conventional STAT3 pathway, i.e. STAT3 tyrosine phosphorylation, dimerization and DNA binding. This pathway, however, may not be the only mechanism through which STAT3 promotes tumorigenesis. For example, forced expression of a nonphosphorylatable STAT3 variant can mimic some STAT3-dependent functions in tumorigenesis⁸⁵. To address this possibility, STAT3 ablation via systemic administration of a validated antisense oligonucleotide was recently tested in mouse tumor models^{79, 86}. The Stat3 antisense oligonucleotide significantly reduced STAT3 protein amounts and inhibited cell proliferation and tumorigenic growth of several human HCC cell lines transplanted into mice⁸⁶. A similar anti-tumor effect of Stat3 antisense oligonucleotides was shown in a mouse lymphoma model⁷⁹. Effective inhibition of tumorigenic growth of many different types of cancer cells transplanted into mice was observed upon treatment with AZD1480, a highly specific JAK2 inhibitor⁸⁷.

### M. Crosstalk Between IKK/NF-κB and STAT3 in Liver Cancer

NF-κB and STAT3 each control the expression of a large number of downstream genes that control cell proliferation, survival, stress responses and immune functions. Some of the target genes for NF-κB and STAT3 overlap and in addition, the two transcription factors are engaged in both positive and negative crosstalk⁸⁸⁻⁹⁰. In mouse DEN-induced HCC, the crosstalk between the NF-κB and STAT3 pathways can be both positive and negative^{39, 49}. DEN-induced hepatocyte death results in release of IL-1α which activates NF-κB signaling in Kupffer cells, which produce a panel of cytokines and growth factors, including IL-6³⁹. IL-6 released by Kupffer cells activates STAT3 in hepatocytes and STAT3-activated genes are critical for compensatory hepatocyte proliferation and liver tumorigenesis^{49, 65}. However, more recently we found that the two transcription factors are also engaged in negative crosstalk within HCC cells⁶⁵. NF-κB activation results in increased expression of proteins, such as ferritin heavy chain and superoxide dismutase 2 that have an anti-oxidant function that prevents excessive ROS acccumulation^{41, 42}. Inactivation of IKKβ in HCC cells or hepatocytes favors the accumulation of ROS which oxidize the catalytic cystein of various protein tyrosine phosphatases (PTPs)⁴¹, including SHP1 and SHP2, the phosphatases that dephosphorylate STAT3 and JAK2⁹¹. Oxidation of SHP1 and SHP2 results in loss of their catalytic activity and accumulation of phosphorylated and activated JAK2 and STAT3, which stimulate the proliferation and tumorigenic growth of NF-κB-deficient HCC⁶⁵. Treatment of mice bearing IKKβ-deficient tumors with an anti-oxidant (BHA) restores SHP1/2 activity, reduces JAK2 and STAT3 phosphorylation and inhibits tumor growth. More recently, the loss of IKKβ in neutrophils was also found to result in activation of STAT3, which enhances the survival and proliferation of NF-κB-deficient neutrophils⁹².

Not only NF-κB can affect STAT3 activity, STAT3 was found to contribute to NF-κB activation. Activated STAT3 in cancer cells is able to bind RelA/p65 in the nucleus and this results in reversible acetylation of RelA/p65 by the STAT3-recruited acetyltransferase p300⁹³. Acetylation of RelA/p65 prolongs its nuclear retention⁹⁴. Therefore, it was suggested that activated STAT3 may account for constitutive activation of NF-κB in some human cancers. This mechanism, however, does not seem to operate in most human HCCs as the majority of tumors with activated STAT3 do not show NF-κB activation⁶⁵.

Based on the above discussion in sections E-F, although the etiology of human HCC is well established, its molecular pathogenesis is poorly understood. As a consequence, mechanism-based therapies for HCC are rare and being refractory to conventional anti-cancer drugs, HCC remains to be one of the deadliest human cancers with a 5 year survival rate of less than 10 percent⁹⁵. The studies discussed above suggest that NF-κB and STAT3 are likely to play important roles in liver inflammatory responses and maintenance of homeostasis and also make critical contributions to HCC development and progression. Although the mechanisms responsible for NF-κB and STAT3 activation in human HCC are not fully understood, a role for NF-κB regulated expression of the STAT3-activating cytokine IL-6 has recently emerged both in viral hepatitis and in hepatosteatosis⁹⁶. Both the pathways that control IL-6 expression and those that control its ability to activate STAT3 offer interesting opportunities to therapeutic intervention as well as prevention.

A variety of animal models were used to study the roles of NF-κB, STAT3 and other signaling pathways in HCC development. However, due to the inability of human hepatitis viruses to infect mice or rats, a rodent model for virally-induced hepatocarcinogenesis is still not available. In addition, most of our mechanistic understanding of NF-κB and STAT3 in HCC comes from studies using cell type-specific knockout mice. NF-κB or STAT3 in these mice are ablated only in certain cell types and remain intact and fully functional in most other cells. Thus, the results obtained may not precisely predict the effect of pharmaceutical inhibitors that interfere with the activity of these transcription factors in all cells. The successful translation of the knowledge gained about NF-κB and STAT3 in HCC will depend on suitable solutions to these potential problems and appropriate human studies that will validate the promising results obtained in mice.

### N. Discussion of Results in Examples 12-16.

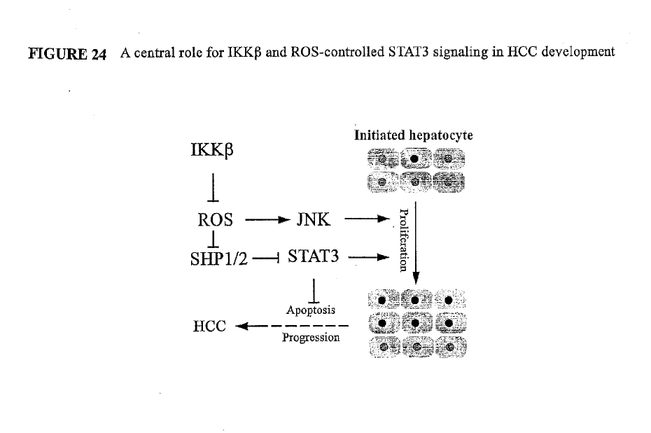

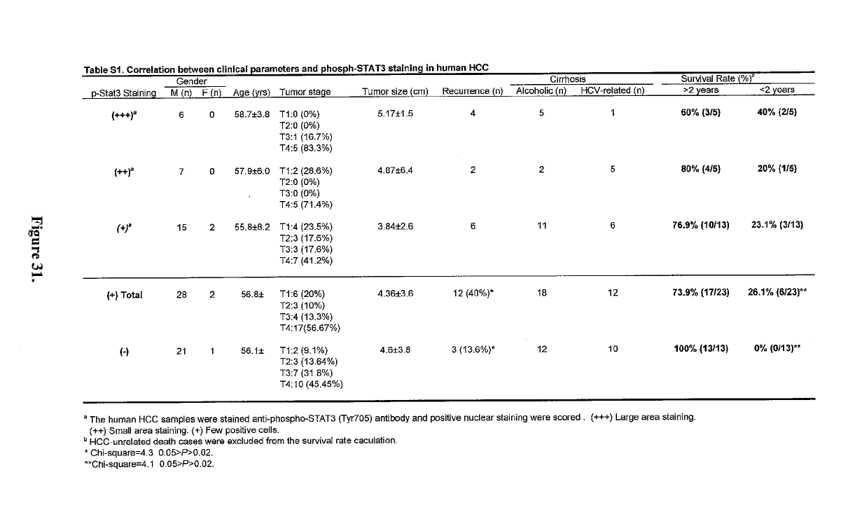

We have developed an experimental system based on transplantation of DEN-initiated hepatocytes into MUP-uPA mouse liver that allows one to examine factors and mechanisms that affect the progression of initiated, pre-neoplastic, hepatocytes into full-blown HCC. Using this system, we found that loss of IKKβ in initiated hepatocytes greatly enhances HCC development even when IKKβ is deleted or inhibited many months after tumor initiation. Previously, IKKβ and its regulatory subunit NEMO/IKKγ were found to negatively control HCC development in models that depend on compensatory proliferation triggered by chemically-induced or spontaneous hepatocyte death and liver injury (Luedde et al., 2007; Maeda et al., 2005). This inhibitory effect on HCC development was proposed to be exerted during early tumor promotion and be due to loss of NF-κB pro-survival activity, which is more severe in Nemo/Ikkγ^{Δhep} than in Ikkβ^{Δhep} mice (Karin, 2006). The current results, however, show that IKKβ-driven NF-κB also inhibits late tumor promotion and progression of initiated hepatoma cells through effects on ROS metabolism that exert a negative control over the STAT3 signaling pathway (FIG. 24). STAT3 itself is frequently activated in human HCCs, especially in aggressive tumors with poor prognosis (Calvisi et al., 2006; FIG. 31 (Table S1)) and we now show that STAT3 activation is subject to negative regulation by NF-κB and is essential for HCC induction. The inverse relationship between NF-κB and STAT3 also applies to a major sub-fraction of human HCCs.

The ability to temporally and physically separate tumor initiation, which in our case occurs upon DEN-induced mutagenesis, from late tumor promotion and malignant progression, has been instrumental to the success of this study. Interestingly, transplantation of DEN-initiated hepatocytes from either a male or female donor to normal C57BL/6 recipients, as opposed to MUP-uPA mice, has never given rise to detectable HCCs. These findings underscore the importance of the microenvironment and circulatory system in tumor progression and malignant conversion. Although the exact factors responsible for the permissive nature of the MUP-uPA liver microenvironment remain to be determined, it should be noted that MUP-uPA mice experience chronic low grade liver injury accompanied by a small, but significant, elevation in IL-6 expression and ROS accumulation and eventually develop low grade liver fibrosis. These are the same kind of changes that accompany the development of human HCC.

Using the transplant system, as well as a culture system that allowed the derivation of the dih cell strains described above, we found that deletion or inhibition of IKKβ in initiated hepatocytes or HCC-derived cells increases their proliferative and tumorigenic potential. The effect is due to loss of NF-κB activity, because specific NF-κB inhibition through expression of IκB super-repressor results in a similar effect. Similar findings were made in squamous cell carcinoma (SCC), where NF-κB was shown to inhibit keratinocyte proliferation and Ras-induced tumorigenesis through negative regulation of JNK activity, whose exact mechanism was not identified (Dajee et al., 2003; Zhang et al., 2004). We now show that another way through which NF-κB inhibits proliferation and tumorigenesis is negative regulation of STAT3 activation. As shown previously for JNK in TNF-α-treated NF-κB-deficient cells (Kamata et al., 2005), enhanced STAT3 activation in Ikkβ^{Δ} cells or tumors is due to oxidative inhibition of PTPs, whose catalytic cysteine is extremely susceptible to oxidation (Meng et al., 2002; Salmeen et al., 2003). While previous work has shown that PTPs are oxidatively inactivated under rather harsh conditions which favor ROS accumulation, such as TNF-α-induced cell death (Kamata et al., 2005), the present work shows that significant PTP inhibition and subsequent kinase activation occur under relatively normal conditions, as long as NF-κB-dependent anti-oxidant defenses (Kamata et al., 2005; Pham et al., 2004) are dismantled. The anti-oxidant function of NF-κB, which is exerted in part through expression of ferritin heavy chain and superoxide dismutase 2 (Kamata et al., 2005; Pham et al., 2004), is particularly important in the liver, an organ that is heavily engaged in oxidative metabolism. Indeed, the deletion of hepatocyte NEMO/IKKγ results in spontaneous liver damage, hepatosteatosis, fibrosis and HCC formation, all of which can be prevented by administration of an anti-oxidant (Luedde et al., 2007). While our work demonstrates an essential and critical role for STAT3 in HCC development and progression, STAT3 has been known to be critically involved in several other malignancies, including SCC (Chan et al., 2004) and CAC (Bollrath et al., 2009; Grivennikov et al., 2009) and JAK2 or STAT3 inhibitors were found to inhibit the growth of several human cancers (Hedvat et al., 2009). Notably, we detected phosphorylated (i.e. activated) STAT3 in approximately 60% of human HCCs, with STAT3-positive tumors being more aggressive. These findings are consistent with those of other studies in which STAT3 was found to be activated in the majority of HCCs with poor prognosis and not in surrounding non-tumor tissue or normal liver (Calvisi et al., 2006; Lin et al., 2009). Expression of the STAT3 activating cytokine IL-6 is elevated in both liver cirrhosis and HCC (Tilg et al., 1992; Trikha et al., 2003) and was recently found to correlate with rapid progression from viral hepatitis to HCC (Nakagawa et al., 2009; Wong et al., 2009). In addition, activating mutations in the gene encoding the gp130 signaling subunit for IL-6 and other cytokine receptors were found to account for benign hepatic adenomas (Rebouissou et al., 2009). When combined with a β catenin activating mutation, these mutations which result in STAT3 activation lead to HCC development. Our findings suggest that NF-κB may also be engaged in negative regulation of STAT3 in a sub-fraction of human HCCs as the frequency of STAT3-positive HCCs is two-fold lower in NF-κB-positive HCCs than in NF-κB-negative HCCs. Altogether, there is little doubt that STAT3 is a key regulator of liver tumorigenesis in mice and men. Our results suggest that in addition to its role in early tumor development, where it suppresses apoptosis and enhances proliferation of pre-neoplastic cells (Bromberg and Wang, 2009), STAT3 plays a critical role during tumor progression when it is activated many months after the HCC initiating event caused by DEN exposure. We identified the IKK-NF-κB axis as a negative regulator of STAT3 activation, but it needs to be determined how NF-κB activity is downregulated during human hepatocarcinogenesis and evaluate its relative contribution to STAT3 activation vis a vis the effects of cytokines and growth factors. We also show that STAT3 can be activated in response to ROS accumulation and it should be noted that several of the most prominent HCC risk factors, including HCV and hepatosteatosis, result in oxidative stress (El-Serag and Rudolph, 2007; Parekh and Anania, 2007; Wang and Weinman, 2006).

The present work as well as previous work with Ikkβ^{Δhep} and Nemo/Ikkγ^{Δhep} mice (Luedde et al., 2007; Maeda et al., 2005; Sakurai et al., 2006) demonstrate an anti-tumorigenic role for NF-κB in hepatocytes. However, it was also described that NF-κB activation promotes hepatocarcinogenesis in Mdr2^{-/-} mice, which develop cholestatic hepatitis and eventually liver cancer (Pikarsky et al., 2004), and in transgenic mice that overexpress lymphotoxin (LT)αβ in their liver (Haybaeck et al., 2009). Notably in both Mdr2^{-/-} and LTαβ-transgenic mice, HCC development depends on chronic low grade inflammation and no liver injury has been observed either prior to or subsequent to NF-κB inhibition (Haybaeck et al., 2009; Pikarsky et al., 2004). Most likely in these models, in contrast to the injury-driven Ikkβ^{Δhep}+DEN and Nemo/Ikkγ^{Δhep} models, the main function of NF-κB in hepatocytes is to upregulate the expression of chemokines needed for recruitment of inflammatory cells that contribute to the microenvironment in which these tumors develop. In the Nemo/Ikkγ^{Δhep} model, however, the absence of hepatocyte NF-κB results in ROS accumulation and its sequela: chronic liver damage, hepatosteatosis, fibrosis and eventual HCC development, whereas in the transplantation model described above IKKβ-deficient and -proficient initiated HCC cells are placed into a permissive microenvironment created by chronic proteolytic damage to the liver. In human hepatocarcinogenesis, which is mainly caused and promoted by chronic HCV or HBV infections or by hepatosteatosis, both inflammatory and injury/repair responses are likely to be involved (Berasain et al., 2009). In addition, some viruses inhibit NF-κB activation to facilitate cell killing and avoid immune detection (Roulston et al., 1999). Therefore, all of the above model systems bear some relevance to human HCC development and given the complex effects of IKK/NF-κB inhibition, a more attractive and likely candidate for therapeutic targeting is the STATS signaling pathway.

O. Methods for Determining Progression of HICs into HCCs

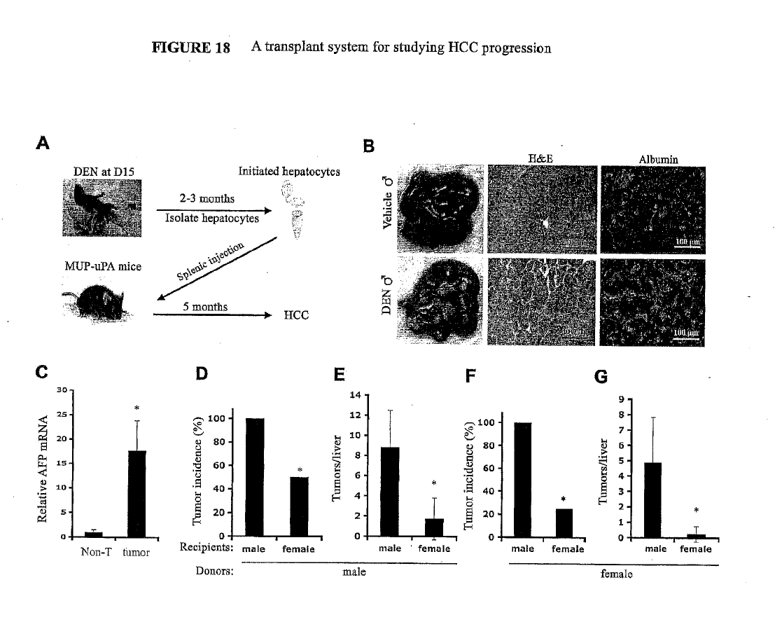

The invention provides a method for determining progression of hepatocellular carcinoma initiating cells (HICs) into hepatocellular carcinoma (HCC) cells, comprising a) administering diethyl nitrosamine (DEN) to a C57BL/6 mouse to produce a donor mouse, b) isolating a population of hepatocyte cells from the donor mouse, c) introducing the isolated hepatocyte cell population into the liver of a MUP-uPA transgenic mouse to produce a treated mouse host, and d) determining the presence of HCC in the liver of the treated mouse host, wherein detection of HCC determines progression of HICs in the isolated hepatocyte cell population into HCC cells (Example 12, FIG. 18). In one embodiment, the HCC cells in the liver of the treated mouse host express increased levels of albumin compared to control non-tumor cells (Example 12, FIG. 18B) and/or express increased levels of α-fetoprotein compared to control non-tumor cells (Example 12, FIG. 18C). In a particular embodiment, the treated mouse host is male (Example 12). In a more particular embodiment, the male treated mouse host comprises a higher number of HCC tumors than the number in a control female treated mouse host (Example 12, FIG. 18D, F) and/or the male treated mouse host comprises a higher number of tumors per liver than the number in a control female treated mouse host (Example 12, FIG. 18E, G). In a further embodiment, the C57BL/6 donor mouse is female (Example 12).

### EXPERIMENTAL

### Example 1

### DEN-Induced HCC in Mouse Model of Cirrhosis

We have used the hepatic pro-carcinogen DEN (diethyl nitrosamine), which undergoes metabolic activation in zone 3 (highly differentiated) hepatocytes³³ to induce HCC in mice. DEN effectively induces HCC in BL6 mice³⁴, the most common genetic background for gene disrupted mice. Most aspects of HCC induction by DEN in mice are quite similar in their underlying pathogenic mechanisms to human HCC. Most importantly, HCC induction by DEN does not solely rely on its ability to induce mutations, but is also dependent on induction of liver damage and subsequent compensatory proliferation³⁴⁻³⁶. Induction of liver damage by DEN depends on ROS accumulation, a factor suggested to be a major contributor to the pathogenesis of human HCC^{1, 37}. DEN-induced ROS accumulation is confined to zone 3 hepatocytes^{34, 36}, the cells that express the DEN-activating enzymes Cyp2E1 and Cyp2A5³³, suggesting that HIC and HCC originate from these cells. Like human HCC, DEN-induced HCC shows a marked gender bias that depends on elevated IL-6 production and subsequent STAT3 activation in male mice³⁸. We also demonstrated that the ability of DEN to induce HCC in mice is strongly potentiated by obesity through a mechanism dependent on enhanced TNF and IL-6 signaling, which promote development of hepatosteatosis³⁹. A recent review article in Cancer Cell has acknowledged the similarity of our findings to clinical observations made in human HCC and highlighted their translational relevance⁴⁰. We also showed that DEN-induced HCC depends on activation of STAT3, the transcription factor that is most frequently activated in human HCC²⁴.

### Example 2

### Isolation and Characterization of HIC Using DEN-Treated Mouse Model of Cirrhosis

To investigate the contribution of STAT3 and other mechanisms to HCC progression, we have developed a transplant system based on introduction of hepatocytes isolated from DEN-treated or control mice into MUP-uPA mice via intrasplenic injection²⁴. Due to expression of urokinase plasminogen activator (uPA), the MUP-uPA liver is subject to chronic damage and compensatory proliferation⁴¹ and exhibits low grade fibrosis²⁴, thereby allowing proliferation of transplanted hepatocytes. As described herein, we noted that instead of yielding uniform suspensions of single hepatocytes as it does in PBS-injected mice, collagenase digestion of DEN-treated livers generates a small number of collagenase-resistant, tightly-packed aggregates of small hepatocytes in addition to well dispersed, single hepatocytes (FIG. 8a).

We developed methods for separating aggregated from non-aggregated cells and by transferring the cells to MUP-uPA mice, found the aggregates almost 20-fold more potent in initiating HCC than non-aggregated hepatocytes from same liver (FIG. 8b). Importantly, aggregated hepatocytes do not give rise to HCC when injected intrasplenically or subcutaneously into normal BL6 mice and thus differ from fully malignant HCC cells²⁴. In addition, aggregated hepatocytes injected into MUP-uPA mice only give rise to HCC nodules after a 5-month latency and no tumors were ever detected at the site of injection, the spleen. MUP-uPA mice injected with hepatocytes from PBS-treated mice never develop HCC, although when they are old, they exhibit a small number of benign hepatic adenomas²⁴.

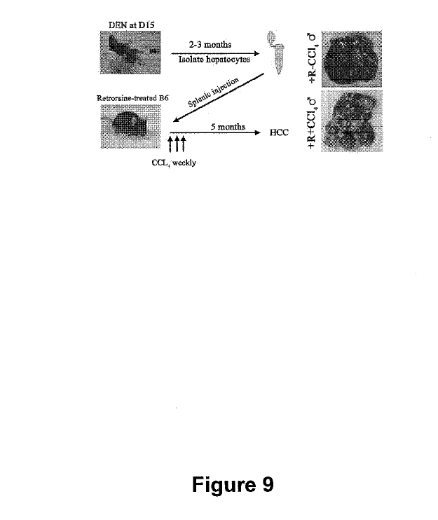

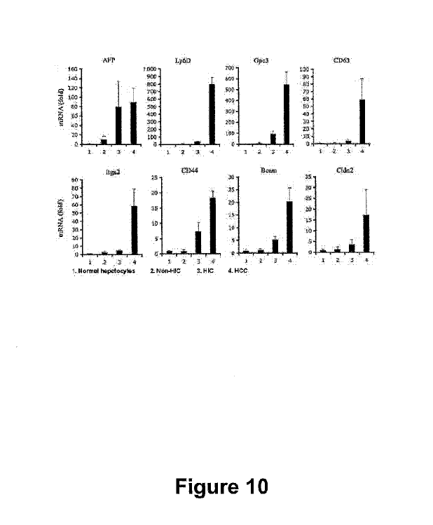

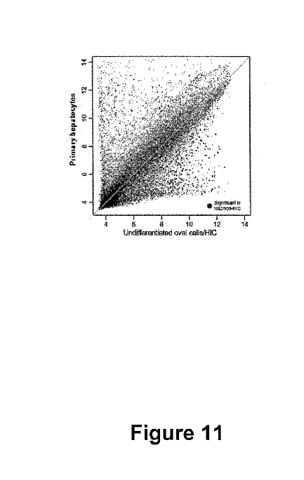

Although aggregated hepatocytes do not form tumors in BL6 mice, they form HCCs when injected into BL6 mice that were pre-treated with retrorsine to inhibit proliferation of endogenous hepatocytes⁴² followed by repetitive carbon tetrachloride (CCl₄) treatments post-transplantation to induce compensatory proliferation (FIG. 9). Omission of CCl₄ prevents tumor induction and extensive genotyping reveals that in both MUP-uPA and retrorsine+CCl₄-treated mice, all HCCs originate from transplanted cells, although host-derived cells especially myeloid cells, are recruited into the tumors. Expression profiling revealed that aggregated hepatocytes differ in their gene expression profile from non-aggregated hepatocytes (FIG. 10). Interestingly, many genes that distinguish aggregated from non-aggregated hepatocytes overlap with genes that distinguish oval cells from normal hepatocytes (FIG. 11), suggesting that aggregated hepatocytes are related to oval cells. Indeed, oval cells induced by bile duct ligation (BDL) or choline deficient diet (CDE) form collagenase-resistant aggregates, but these aggregates have not formed HCCs in transplanted mice. Furthermore, there are no published reports regarding oval cells tumorigenicity. These results suggest that either HIC and oval cells share a common origin or that oval cells migrating from periportal areas associate with DEN-induced HIC originating at zone 3 (peri-centrally).

The results indicate that we have succeeded for the first time in isolating pre-malignant hepatocytes from precancerous livers and have unequivocally established their ability to give rise to HCC in transplanted mice that provide a liver microenvironment supportive of malignant progression. We therefore named the HCC progenitors present within hepatocyte aggregates-HCC initiating cells or HIC.

### Example 3

### Isolation and Characterization of HIC Using Tak1^{Δhep} Mouse Model of Cirrhosis

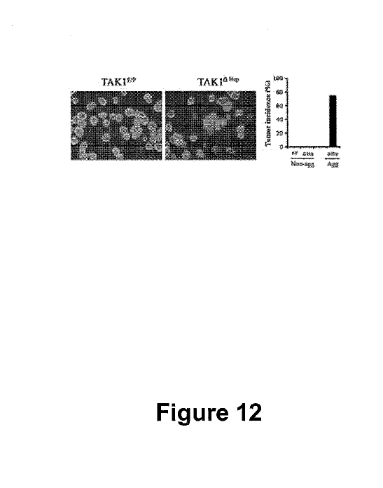

Being concerned that HIC are unique to the DEN model, we asked whether such cells can be isolated from other HCC models, such as Tak1^{Δhep} mice. Hepatocyte-specific TAK1 ablation results in spontaneous liver damage, inflammation, fibrosis and eventual development of HCC⁴³, a sequence that is similar to the common pathogenic sequence of human HCC, and the major reason for selecting this model for further studies. Hepatocyte suspensions from 1 month old Tak1^{Δhep} mice contained aggregates absent in control Tak1^{F/F} mice, and these aggregates generated HCC in MUP-uPA mice after 5 months, but non-aggregated hepatocytes were not tumorigenic (FIG. 12). Thus, we had established reliable, robust and generally applicable methods for identification and isolation of pre-malignant hepatocytes, which as discussed herein, are likely to be derived from precancerous lesions.

### Example 4

### Isolation and Characterization of HIC Using Tak1^{Δhep}/p38α^{Δhep} Mouse Model of Cirrhosis

We have also developed mice in which both TAK1 and p38a are ablated in hepatocytes. Many of these mice, Tak1^{Δhep}/p38α^{Δhep}, develop severe bridging fibrosis that resembles human cirrhosis. These mice are used as a mouse model for cirrhosis.

### Example 5

### Characterization of HICs

We developed a system for following the malignant progression of cells isolated from livers of DEN-treated mice²⁴. We perfused livers of PBS- or DEN-injected mice with a collagenase solution to prepare hepatocyte suspensions that were injected intrasplenically into 5 weeks old BL6 MUP-uPA mice, whose livers are subject to chronic regeneration due to uPA expression⁴¹. Within 5 months, transplanted hepatocytes originating from DEN-treated mice gave rise to HCC in the recipients' livers²⁴. Importantly, hepatocytes from PBS-treated controls never gave rise to cancer and hepatocytes from DEN-treated mice do not generate tumors at the site of injection—the spleen.

We noted that hepatocyte preparations from DEN-treated mice contain aggregates that are not present in PBS-treated mice (FIG. 8a) and developed a method for separating aggregated from non-aggregated hepatocytes. Aggregated cells were far more potent in HCC initiation than non-aggregated cells (FIG. 8b). DEN-induced aggregated hepatocytes, which contain HIC, do not give rise to HCC when transplanted into normal BL6 mice, unless such mice are treated with retrorsine to block endogenous hepatocyte proliferation and challenged with CCl₄, after transplantation, to induce compensatory proliferation of transplanted cells (FIG. 9).

Using HIC isolated from DEN-treated GFP-expressing mice, we demonstrated that the HCCs are derived from the transplanted cells, and confirmed these findings by extensive genotyping of isolated tumors.

Characterization of aggregated hepatocytes by whole genome microarrays and Q-RT-PCR of individual genes revealed that the HIC transcriptome is distinct from that of normal hepatocytes but is closely related to that of fully malignant HCC (FIG. 10). Aggregated hepatocytes express high amounts of the HCC marker α-fetoprotein (AFP), but express lower amounts of CD44, a marker for HCC stem cells^{10,53,54}, which is not expressed by normal hepatocytes. While not limiting the invention to a particular mechanism, the quantitative differences between HIC and HCC could be due to the heterogeneous and impure nature of the former. Curiously, many of the expressed genes that distinguish HIC from normal hepatocytes overlap with genes whose expression distinguishes oval cells from normal hepatocytes (FIG. 11). However, the HIC and oval cell transcriptomes are not identical and the differences may underlie their different tumorigenic potentials.

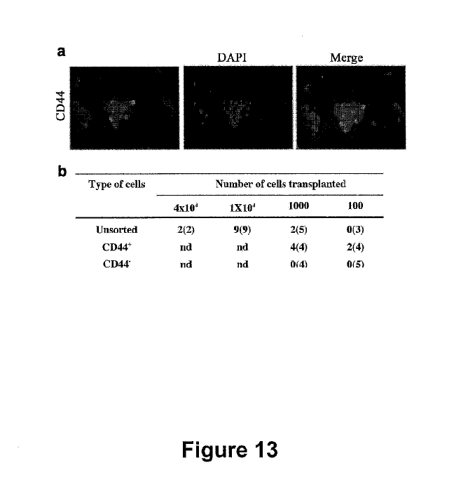

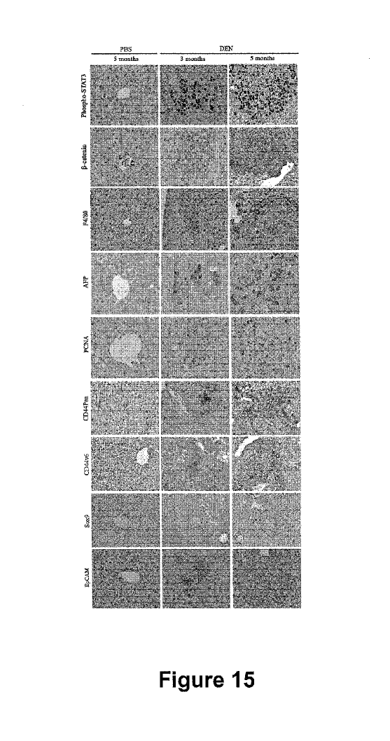

Analysis of CD44 expression by immunofluorescence revealed that not all aggregated cells are CD44⁺ (FIG. 13a). We therefore separated dispersed aggregates into CD44⁻ and CD44⁺ cells and found that only CD44⁺ cells generated HCC in MUP-uPA mice (FIG. 13b). Remarkably, as few as 100 CD44⁺ cells can produce HCC. HIC display down-regulation of genes involved in drug metabolism that are characteristic of differentiated zone 3 hepatocytes. Collectively, these results indicate that HIC may originate from zone 3 hepatocytes by dedifferentiation or through expansion of hepatoblasts, the earliest cell type that expresses AFP and EpCAM⁵⁵, which is another marker expressed by HIC (FIG. 15). Alternatively, HIC may be derived from oval cells or the aggregates are formed by an interaction between oval cells and zone 3-derived HIC. Notably, hepatoblasts are rare cells in postnatal livers that reside mainly within canals of Hering⁵⁶, rather than pen-centrally. Likewise, oval cells originate from a pen-portal location in response to injuries induced by BDL, CDE, diet supplemented with 3-diethoxycarbonyl-1,4-dihydrocollidine (DDC) and 2-acetylaminofluorene (2-AAF)⁵⁷. Neither DEN nor CCl₄ are known to induce oval cells.

To ensure that aggregates containing HIC are not unique to DEN-treated mice, we examined their presence in Tak1^{Δhep} mice, which spontaneously develop HCC after undergoing chronic liver damage, inflammation and fibrosi⁴³. Digests of Tak1^{Δhep} livers contained collagenase-resistant aggregates that were not present in Tak1^{F/F} mice, which do not develop HCC (FIG. 12). When transplanted into MUP-uPA mice, only aggregates generated HCC.

We examined the relationships between HIC-containing aggregates and FAH. The latter appear within zone 3, as early as 3 months after DEN administration into male mice, but their appearance and growth are delayed in females (FIG. 14). BrdU labeling indicates that proliferative hepatocytes in DEN-treated livers are largely confined to FAH (FIG. 14). Furthermore, only aggregated hepatocytes contained BrdU⁺ cells. FAH also contain cells positive for AFP, EpCAM, CD44 and the proliferation marker PCNA, as well as cells with activated STAT3, partially activated β-catenin and nuclear Sox9 (FIG. 15). While not limiting the invention to a particular mechanism, these results suggest that HIC are derived from FAH.

### Example 6

### a. Compare the Gene Expression Profile that Defines DEN-Induced HIC to that of HIC Induced by Loss of TAK1 and p38 MAP Kinase (MAPK). Analyze the Transcriptomes of CD44⁺ and CD44⁻ Cells in HIC Aggregates Isolated from Different Mouse Models

We identified pre-malignant hepatocytes in DEN-treated mice and isolated them as aggregates refractory to collagenase digestion. Aggregated hepatocytes are far more capable of producing HCC in transplanted mice than non-aggregated cells and were termed HIC. Nonetheless, the aggregates are heterogeneous and only their CD44⁺ cells are tumorigenic. Transcriptome analysis indicates that DEN-induced HIC differ from normal hepatocytes, but are somewhat similar to oval cells/adult liver stem cells which are non-tumorigenic. HIC are also present in Tak1^{Δhep} mice, which spontaneously develop HIC subsequent to chronic hepatitis and fibrosis. We will use gene expression profiling to compare DEN-induced HIC to those induced by TAK1 deficiency and to oval cells. Since mice lacking both TAK1 and p38 exhibit severe fibrosis that approaches human cirrhosis, we will also isolate HIC from these mice and compare them to the other models. We will determine the importance of CD44 in TAK1-deficient HIC and conduct transcriptome analysis on all 3 models to identify CD44-associated HIC marker genes.

We isolated aggregates containing HIC from livers of DEN-treated male mice and compared their gene expression profile to normal hepatocytes from PBS-treated livers, non-aggregated hepatocytes isolated from same DEN-treated mice and DEN-induced HCC. Cancer cells were isolated by perfusion/collagenase digestion of large tumors from 9 months old DEN-treated males²⁴, whereas the different hepatocyte types were prepared by collagenase perfusion/digestion of livers and separated as described above into aggregated and non-aggregated cells. Total RNA was extracted from approximately 10⁶ cells per experiment with RNeasy kit (Qiagen). After confirming quality and purity, RNA was converted to biotinylated cRNA using Illumina RNA amplification kit according to manufacturer's instructions. Labeled cRNA was hybridized to Illumina Mouse 6 Sentrix Expression Bead Chip and data analysis and quality control were conducted using BeadStudio software (Illumina) at the BioGem core facility at UCSD. Heat maps provided us with lists of genes and gene groups whose expression was different between HIC and normal hepatocytes and between HIC and HCC cells. Further analysis was conducted using R environment and the Limma package followed by functional characterization with the DAVID^{58,59} program and Ingenuity Pathway Analysis (IPA).

The results show that HIC are clearly different from normal hepatocytes (FIG. 11), whereas non-aggregated hepatocytes isolated from livers of DEN-treated mice are essentially identical to normal hepatocytes from PBS-injected mice after correction for contamination by HIC/aggregates, which approximates 5%. Correction for HIC contamination by normal hepatocytes, which can reach 30%, indicates that the HIC transcriptome is very similar to the DEN-induced HCC transcriptome. Curiously, many genes whose expression distinguishes HIC from normal hepatocytes overlap with published genes that distinguish oval cells from normal hepatocytes⁶⁰ (FIG. 11). We used Q-RT-PCR with specific primer sets to compare the relative expression of a small number of marker genes between the four cell populations (FIG. 10). We will expand the number of genes subjected to such analysis. Those genes that are confirmed to be over-expressed in HIC relative to normal hepatocytes will be subjected to more detailed time course studies by Q-RT-PCR and in situ hybridization (ISH), using established procedures, in livers isolated from DEN-treated, Tak1^{Δhep} and Tak1^{Δhep}/p38α^{Δhep} mice at different time points after DEN administration or birth.

Since DEN-induced HIC may differ in their origin and mechanism of induction from HIC in other HCC models, we will conduct similar transcriptomic analysis on aggregated and non-aggregated hepatocytes from Tak1^{Δhep} (FIG. 9) and Tak1^{Δhep}/p38α^{Δhep} mice. These models are chosen because their HCC development follows a sequence of tissue injury, hep inflammation and fibrosis⁴³, similar to that of human HCC⁶¹. Furthermore, Tak1^{Δhep}/p38α^{Δhep} double mutants exhibit more extensive fibrosis than Tak1^{Δhep} single mutants and represent the best mouse model for cirrhosis.

Genes that are overexpressed in Tak1^{Δhep} and Tak1^{Δhep}/p38α^{Δhep} hepatocyte aggregates will be compared to each other and to those that distinguish DEN-induced HIC from normal hepatocytes using R environment, as in FIG. 11. This analysis will reveal whether different HIC types represent small variations of a distinct cell type or could be derived through different mechanisms from distinct origins. The functional significance of any differences will be determined. These experiments will also indicate whether HIC that develop in the context of fibrosis differ from those that appear in non-fibrotic livers and whether more severe bridging fibrosis has any effect on the gene expression profile of pre-malignant hepatocytes.

One goal of these experiments is to identify marker genes that are specific to pre-malignant HIC and learn about the mechanisms that endow HIC with tumorigenic potential. To this end, we will also conduct transcriptomic analysis on oval cells induced by BDL and CDE in the same mouse strain (BL6) used for HCC induction. These experiments, which will identify genes that are unique to DEN-induced HIC and not shared with oval cells, are important because the oval cell transcriptome in FIG. 11 was interrogated in another lab⁶⁰, whose mice may differ from ours. The procedure for oval cell isolation⁶² has been established in our lab. All analyses will be conducted using male mice to eliminate gender-related differences and each set of cells will be isolated from at least 3 individual mice per genotype. Differential expression of selected genes will be confirmed by Q-RT-PCR and ISH as above.

We will subject HIC-specific genes to functional annotation using DAVID™^{58, 59} and IPA software to identify transcription factors/signaling pathways responsible for their activation. This will provide additional tools for HIC identification. Activation of protein kinases and transcription factors first identified by bioinformatics analysis will be confirmed by immunohistochemical (INC) analysis of paraffin-embedded or frozen liver sections from 3 months old DEN-treated mice, 1 month old Tak1^{Δhep} and Tak1^{Δhep}/p38α^{Δhep} mice and 2-3 weeks after BDL or CDE. Tissue sections will be stained with phospho-specific antibodies that detect protein kinase activation (for instance ERK, JNK, p38, IKKβ, TORC1) or transcription factor phosphorylation (phospho-c-Jun, phospho-CREB, phospho-STAT3, etc). We will also use conventional antibodies to detect nuclear accumulation of relevant transcription factors. To confirm activation of individual signaling pathways in HIC-containing FAH in the different models, we will use laser capture microdissection (LCM)⁶³ to selectively excise individual lesions and non-lesion areas from tissue sections. Total RNA isolated from LCM derived cells will be analyzed using RT²Profiler PCR arrays (Qiagen) for activation of different pathways and processes, including but not limited to: Wnt-β catenin, TGFβ, PI3K/AKT, IGFR, Hippo, MAPKs, cell cycle, apoptosis, DNA damage and angiogenesis. Functional analysis of signaling pathways or transcription factors that are unique to HIC will be conducted.

Aggregates from DEN-treated mice are heterogeneous in respect to CD44 expression and only CD44⁺ cells give rise to HCC (FIG. 13). After determining that there is similar heterogeneity in HIC aggregates from Tak1^{Δhep} and Tak1^{Δhep}/p38α^{Δhep} mice, we will separate them into CD44⁺ and CD44⁻ cells as described above (FIG. 13) and examine the two populations for their tumorigenic potential in MUP-uPA mice. We already know that Tak1^{Δhep} aggregates contain CD44⁺ cells. Since one of our goals is to identify a set of cell surface markers useful for reliable HIC identification and isolation and CD44 is a validated marker, we will search for genes that are co-expressed with CD44. We will separate aggregated hepatocytes from the three models, after their dispersion by repeated pipetting, into CD44⁺ and CD44⁻ populations using FITC-conjugated CD44 antibody (Clone IM7, BD553133) and anti-FITC magnetic beads. RNA extracted from the two populations will be subjected to the transcriptomic analyses described above to identify genes that are strongly co-expressed with CD44 and whose differential expression will be confirmed by Q-RT-PCR.

The transcriptomic analysis described above has been useful in identifying relatedness and differences between HIC and oval cells (FIG. 11) and HIC, normal hepatocytes and HCC (FIG. 10). This analysis to provide us with useful information regarding relatedness between DEN-induced HIC and those induced by TAK1 or TAK1+p38α ablation. As all HIC originate from an hepatocytic cell type and give rise to HCC in MUP-uPA mice, they may share many genes that are not expressed in normal hepatocytes. Some of these genes should also be under-expressed in oval cells that lack tumorigenic potential. However, the different HIC types are probably induced through distinct mechanisms. DEN is a carcinogen that also causes acute liver injury and triggers compensatory proliferation³⁵. By contrast, TAK1 ablation causes chronic liver injury, inflammation and fibrosis, but does not constitute an obvious genotoxic insult⁴³. Thus, the origin of HIC in Tak1^{Δhep} or Tak1^{Δhep}/p38α^{Δhep} mice may be more similar to that of human liver pre-malignant cells that appear after chronic inflammation and injury. DEN-induced HIC may be more similar to those induced by exposure to aflatoxin or other carcinogens. Despite their different origins, we will search for markers that are common to all HIC types, which are more likely to be useful for human HIC identification. These markers should be co-expressed with CD44, already found to be expressed in human HCC stem cells^{53,64}. Nonetheless, inclusion of Tak1^{Δhep}/p38α^{Δhep} mice in these studies may lead to identification of cirrhosis-linked HIC markers. However, the fibrotic/cirrhotic microenvironment is tumor promoting due to provision of chemokines that recruit inflammatory/immune cells, as we found in other cancers^{65,66}, rather than causing a major remodeling of the HIC transcriptome. The transcriptomic analysis will identify signaling pathways that may account for HIC formation and maintenance.

### Example 7

### b. Identify Markers for Histochemical HIC Detection in Livers of DEN-Treated, Tak1^{Δhep} and Tak1^{Δhep}/p38α^{Δhep} Mice and Use them to Examine the Kinetics of HIC Appearance and Malignant Progression

The goal of this work is to generate a reliable set of immunohistochemical tools for early detection of HIC and for following their growth and malignant progression. We will use these tools to determine the fate of transplanted GFP-tagged HIC from different models. Results of these experiments will be compared to those of lineage tracing experiments.

Currently, HIC are detected by their ability to form collagenase-resistant aggregates that give rise to HCC after transplantation into MUP-uPA or retrorsine+CCl₄-treated mice. For some applications, this approach is not preferred. IHC-based HIC detection using a unique panel of markers should be easier and more sensitive.

We have already validated CD44 as an HIC marker. DEN-induced FAH contain CD44v6⁺ cells (FIG. 15), but it is not clear whether these cells also express the standard CD44s form detected by the antibody (IM7, BD553133) used for HIC isolation. We will therefore repeat the cell isolation experiments described in FIG. 11 with a CD44v6 specific antibody (ABD Serotec MCA1967) and perform flow cytometric analysis using an Accuri C6 instrument to determine whether the CD44s and CD44v6 antibodies recognize the same cell population.

CD44s expression was also detected in oval cells⁶⁸, but it is not known whether these cells express the CD44v6 isoform. We will therefore compare CD44v6 expression in HIC and oval cells induced by BDL or CDE. Next, we will analyze CD44 co-expressed HIC-specific genes for those that encode cell surface proteins and other abundantly expressed, non-secreted, molecules to identify additional HIC markers. One such molecule is EpCAM, but we also don't know if it is co-expressed with CD44. We will therefore screen commercially available antibodies directed against candidate HIC markers for their ability to co-stain CD44⁺ HIC from different sources using two-color flow cytometry. Since this analysis is mainly useful for surface staining, we will also examine the different antibodies for staining of cytospin preparations of CD44⁺ HIC. Stained cells will be analyzed using indirect immunofluorescence on a Leica TCS SPE-II confocal microscope. To confirm that any new cell surface marker thus identified is unique to HIC, we will use it to repeat the cell separation and transplantation experiments described in FIG. 13 with dispersed hepatocyte aggregates from DEN-treated, Tak1^{Δhep} and Tak1^{Δhep}/p38α^{Δhep} mice.

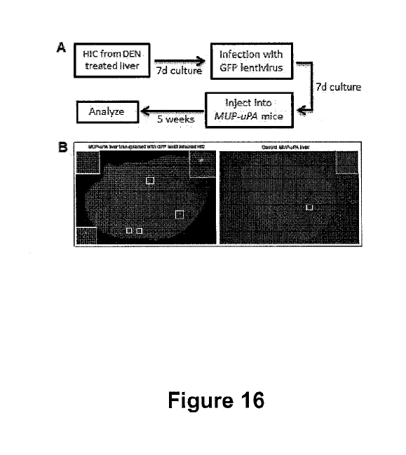

The suitability of any antibody for cell isolation or flow cytometry does not guarantee its suitability for immunohistochemical (IHC) analysis using paraffin-embedded (1^{st} priority) or frozen sections. For instance, we searched through several commercially available CD44 antibodies, until we found one (ABD Serotec MCA4703) that is suitable for IHC. If no commercial antibodies to additional HIC markers appropriate for IHC are found, we will subclone the cDNA encoding the relevant antigen into a bacterial expression vector to produce an immunogen that will be used to generate new polyclonal and monoclonal antibodies. All antibodies will be evaluated for their suitability for IHC analysis. These experiments will provide a panel of markers, including CD44s and/or CD44v6, that will allow unequivocal identification of HIC in the three mouse models (i.e., DEN-treated mice, Tak1^{Δhep} mice, and Tak1^{Δhep}/p38α^{Δhep} mice) and their distinction from non-tumorigenic oval cells. Once the proper marker panel is identified, we will examine the kinetics of HIC appearance in livers of DEN-treated, Tak1^{Δhep} and Tak1^{Δhep}/p38α^{Δhep} mice. To follow the kinetics of progression to HCC, we will inject HIC isolated from the three models that have been transduced with an eGFP-expressing lentivirus as outlined in FIG. 16 into MUP-uPA mice. The fate of the introduced cells will be followed by fluorescent microscopy of frozen sections and by staining paraffin-embedded sections with GFP antibodies at 3, 7, 10 and 14 days and 1-5 months after transplantation. Frozen and paraffin-embedded liver sections will also be stained with HIC marker antibodies.

Antibodies to cell surface proteins that are co-expressed with CD44s and/or CD44v6 should be suitable for magnetic bead isolation of HIC and will provide us with a panel for unequivocal identification of HIC in mouse livers, preferably where at least one marker is not strongly expressed by oval cells. We will develop antibodies to cell surface proteins that are co-expressed with CD44s and/or CD44v6 using well established methods. Once suitable antibodies are available, we will perform the described analyses. GFP-tagged HIC introduced into MUP-uPA mice form clusters of proliferating cells rather quickly (FIG. 16). These cells should retain expression of HIC markers, but at some point may upregulate markers that are linked to HCC progression, such as Ly6D and Gpc3. Expression of these markers should correlate with acquisition of HCC-like morphology.

### Example 8

### c. Conduct Signaling and Lineage Tracing Studies to Explore the Relationships Between HIC and Oval Cells and Identify the Origin of HIC in Different Mouse HCC Models

Many genes that distinguish DEN-induced HIC from normal hepatocytes overlap with those that distinguish oval cells from normal hepatocytes. However, DEN does not induce an oval cell reaction and oval cells are thought to be non-tumorigenic. Nonetheless, many DEN-induced HIC express Sox9, a transcription factor that is also expressed in oval cells, adult liver progenitors, periportal hepatocytes and embryonic ductal plate cells. We will conduct signaling studies and use Sox9 to identify the origin of HIC in different HCC mouse models, determine their relationship to the above cell types and the importance of Sox9 and other pathways in their induction.

To identify signaling pathways that drive HIC formation, control their progression to a more malignant phenotype and distinguish them from oval cells, which are not malignant, we will identify these pathways to provide a better understanding of HIC origin and induction, since their targeting may allow specific eradication of pre-neoplastic HIC before they progress to HCC. The data suggest that both STAT3 and β-catenin are activated in DEN-induced HIC (FIG. 15) and further experiments will tell us whether TAK1-deficient HIC also exhibit STAT3 and β-catenin upregulation. STAT3 and β-catenin, however, are also involved in oval cell generation⁵⁷ and STAT3's role in HCC development was extensively studied^{24,27}, and so was β-catenin's role in liver regeneration and injury repair⁶⁹. Whereas β-catenin activating mutations occur in HCC⁷⁰, ablation of the Ctnnb gene in adult hepatocytes enhanced DEN-induced HCC development⁷¹.

To determine whether β-catenin activation is important for HIC maintenance and progression to HCC, we will infect isolated HIC as described in FIG. 16 with a bi-cistronic lentiviral vector we had generated that expresses GFP and the shRNA of the factor to be targeted (in this case (β-catenin) from the histone H1 promoter. β-catenin silenced cells and cells transduced with a “scrambled” shRNA control will be injected into MUP-uPA mice and their ability to generate GFP⁺ nodules that progress to HCC will be examined as above. Similar functional analysis will be conducted on other signaling pathways or transcription factors that are activated in HIC relative to normal hepatocytes and oval cells. We will also determine whether pathways that are selectively activated in different HIC models are more important for progression of the relevant HIC type. In addition to studying pathways that are activated in HIC, we will use lentiviral transduction to fully understand the role of TAK1 in HIC development. Although we believe that TAK1 ablation mainly promotes HIC and HCC development by causing chronic liver injury, this remains to be formally tested as our recent results indicate that NF-κB, one of the main targets for TAK1, is also a cell autonomous inhibitor of malignant progression²⁴, in addition to its well established liver protective function³⁵. Furthermore, the majority of human HCCs are negative for nuclear NF-κB²⁴. To this end, we will transduce Tak1^{Δhep} HIC with bi-cistronic lentiviruses encoding either wild type (wt) TAK1 or a kinase dead mutant. Effects on HIC to HCC progression will be studied as above. We will also use shRNA expressing lentivirus to ablate TAK1 in oval cells and determine whether this provided them with oncogenic potential.

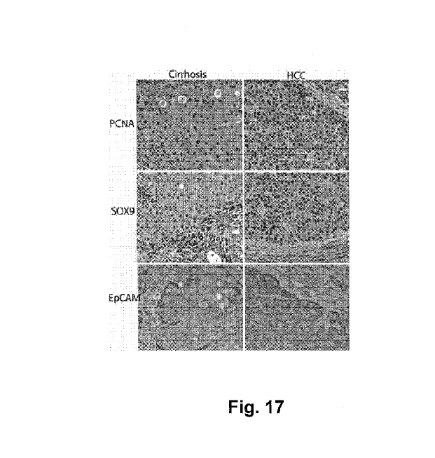

Another transcription factor found to be activated in some cells within FAH is Sox9 (FIG. 15). Sox9 is involved in adult stem cell maintenance^{72,73} and in liver, it is expressed in bipotential hepatobiliary stem cells within intrahepatic bile ducts (IBD)⁶². Sox9 is also expressed in oval cells^{60,74} and its presence in DEN-induced FAH suggests a role for oval cells in FAH formation, even though DEN does not induce oval cells. We also detected Sox9⁺ cells in human cirrhotic nodules and HCC (FIG. 17). To determine the role of Sox9 in HIC induction, we obtained Sox9^{F/F} mice⁷⁵ from Gerd Scherer (Uniklinik Freiburg) and will cross them to Alb-Cre and Tak1^{Δhep}/p38α^{Δhep} mice to generate Sox9^{Δhep} and Tak1^{Δhep}/Sox9^{Δhep} mice. The former will be injected with DEN and HIC development will be examined as above, whereas spontaneous HIC formation in Tak1^{Δhep}/Sox9^{Δhep} mice will be compared to that in Tak1^{Δhep} single mutants. We will also examine whether Sox9 ablation in already formed HIC prevents HIC to HCC progression, by transducing isolated HIC with a bi-cistronic lentiviral vector expressing shRNA to Sox9. These experiments are of importance because Sox9⁺ cells can either be the progenitors for HIC or may interact with them to form tight aggregates that support HIC survival and progression. To better understand the relationships between Sox9 and HIC, we will conduct lineage tracing experiments to determine whether Sox9⁺ cells are the ones that give rise to HIC or whether oval cells are incorporated into FAH but do not evolve into pre-malignant cells. We will deploy Sox9-Cre^{ERT2} mice that express a tamoxifen-regulated Cre^{ERT2} fusion protein from the Sox9 promoter⁷⁶. These mice, obtained from Maike Sander (UCSD), also contain a Rosa26-YFP construct whose expression is blocked by a STOP cassette flanked by IoxP sites. These mice express Cre^{ERT2} only in Sox9⁺ cells, allowing them to be permanently tagged by YFP upon tamoxifen-induced activation of Cre^{ERT2}. We will treat 8 days old Sox9-Cre^{ERT2}/R26-YFP males with tamoxifen to tag IBD Sox9⁺ cells with YFP. At 2 weeks of age, these mice will be given DEN and analyzed by IHC at 3, 5 and 9 months to identify YFP⁺ and Sox9⁺ cells in FAH and HCC. CD44⁺ HIC will be isolated as above and analyzed for YFP expression. Controls will consist of mice of the same genotype treated with either tamoxifen or DEN alone. If CD44⁺ HIC express YFP (and Sox9), they are probably derived from Sox9⁺ cells, but it is plausible that the aggregates will contain YFP⁺ cells that are not CD44⁺ and are therefore non-tumorigenic, although they may support HIC survival and progression. Sox9-Cre^{ERT2}/R26-YFP males will also be injected with DEN when 2 weeks old and given tamoxifen 1-3 months later. Mice will be analyzed by IHC and immunofluorescence to determine whether Sox9⁺ cells migrate into FAH or induced within them at later time points.

STAT3 is important for HCC induction and HIC formation.²⁴ However, the role of β-catenin in HIC induction and progression is more obscure. Based on the requirement for β-catenin in liver development and injury repair⁶⁹, it may be important for HIC generation, but it should be noted that cells within FAH only exhibit partial β-catenin activation (FIG. 15). Nonetheless, as β-catenin signaling has already received considerable attention, we will place most of our efforts on less explored pathways and transcription factors that are preferentially activated in HIC relative to non-malignant hepatocytes and oval cells. Instead of examining the role of different signaling pathways and transcription factors in HIC survival and progression through a knockout approach which is quite time and effort consuming, we will use lentiviral delivery of shRNAs, an approach we have established since the previous submission (FIG. 16). This allows faster and simpler analysis of cell-intrinsic factors that are important for HIC maintenance and progression. Such factors will provide future therapeutic and preventive targets.

Presence of Sox9⁺ cells within FAH is intriguing and deserves further investigation. As discussed above, DEN seems to exert its initial tumorigenic activity within zone 3, whereas Sox9⁺ cells originate from IBD and peri-portal locations, but the source of HIC induced by TAK1 ablation is currently unknown. It is also plausible that in addition to or instead of IBD, Sox9⁺ cells are induced by DEN in zone 3. If such cells give rise to HIC, we will conclude that DEN-induced HIC are derived from de-differentiated hepatocytes. One potential pitfall is a deleterious effect of Sox9 ablation in IBD, resulting in loss of liver mass. If this is evident, we will examine Sox9 function in isolated HIC by shRNA-mediated silencing as described above. Another potential pitfall is incomplete excision of the STOP cassette. This will be addressed by analyzing a few Sox9-Cre^{ERT2}/R26-YFP mice one day after tamoxifen treatment. Interpretation of future results will be based on the percentage of IBD Sox9⁺ cells that are YFP⁺. If YFP⁺ cells are absent in FAH and HCC, Sox9 expression within them is likely to start at a later time point, long after tamoxifen administration.

### Example 9

### d. Investigate Whether Mouse HIC Markers Detect Similar Cells in Human Specimens from Patients with Underlying Liver Diseases Associated with High Risk Of HCC. Correlate the Frequency of Putative HIC with Disease Severity and Relative HCC Risk and Examine their Tumorigenic Potential in Mice

The goal of this work is to examine whether mouse HIC markers detect putative HIC in livers of patients suffering from pre-cirrhotic and cirrhotic alcoholic and viral hepatitis, NASH and hemochromatosis, chronic liver diseases that are associated with high HCC risk. The frequency of such cells should correlate with disease severity and published relative risk values for each condition and their tumorigenic potential will be confirmed by transplantation into immunocompromised mice.

We will stain formalin-fixed, paraffin-embedded and frozen sections of human liver specimens for validated mouse HIC markers. This will include normal control livers and resected/biopsied liver specimens from patients with pre-cirrhotic and cirrhotic chronic liver disease (CLD), resulting from alcoholic hepatitis, viral hepatitis (HBV/HCV), NASH and hemochromatosis, all of which represent independent HCC risk factors¹. To ensure the analysis of pre-malignant lesions, material containing high-grade dysplastic nodules or frank HCC will be excluded. Biopsies will be confirmed to contain histological atypias representing dysplasia (small cell changes), cirrhotic nodules and low-grade dysplastic nodules. Essentially, the same histological methods used in mice will be used with human material. We have already confirmed presence of EpCAM⁺ and Sox9⁺ cells in human cirrhotic nodules using the same antibodies used for mouse studies (FIG. 17). Many of the other markers were confirmed to be expressed in human HOC. The presence of cells positive for the proper constellation of HIC markers will be correlated with histological features of atypia/dysplasia (small cell change, neovascularization, hepatocytic inclusions, loss of reticulin framework) within the same specimen in close collaboration and consultation with our hepatopathological experts. We will also analyze the specimens for markers of early human HCC, including Gpc3, glutamine synthase (GS) and HSP70. Most importantly, the average frequency of putative HICs within a set of specimens from a particular CLD should correlate with published relative HCC risk factors for that condition¹. For instance, cirrhotic NASH or alcoholic cirrhosis should contain more putative HICs than the pre-cirrhotic stages of the same disease and hemochromatosis specimens should have higher HIC frequency than NASH specimens. Furthermore, more HICs may be found in more advanced CLD based on the Laennec fibrosis scoring system, which correlates with clinical stage and grade of portal hypertension.

If these conditions are met, we will proceed to examine whether the putative HIC are tumorigenic. Cirrhotic human livers will be procured from patients undergoing liver explantation and subsequent orthotopic liver transplantation for end-stage liver disease (ESLD). Patients with solitary or multifocal HCCs will be excluded.

Once human liver tissue from liver tansplantation is procured, it will be transferred on ice for immediate histological analysis and processing by a 24 h/7 d on-call team member. To physically isolate HIC, we will perform collagenase digestion as done in mice²⁴ and has been reported by our group in humans⁷⁷. Under light microscopy, we will determine the relative number, size, and density of aggregates in each liver normalized to the mass of liver tissue employed. The aggregates will be analyzed for expression of the different HIC markers as above. Based on our data herein, we expect human HIC to express CD44s and/or CD44v6. To conclusively determine whether human hepatocytic aggregates contain pre-malignant cells, we will separate aggregated cells from non-aggregated cells using a 70-μm sieve as done in mice. Preparations with viability >80% will be intrasplenically transplanted into 3 weeks old MUP-uPA/NOD-SCID mice, using 10⁵⁻⁷ viable cells in 30 μl of PBS. We will separately transplant each population of cells into 6-12 mice/group/liver depending upon cellular yields. Following transplantation, 2-4 mice/group/liver will be sacrificed 3, 5, or 8 months later to monitor for FAH, dysplastic nodules and HCC development. Blood collected from these mice monthly will be analyzed for presence of human AFP and Gpc3 by ELISA. We will also analyze mouse livers for expression of human Gpc3 and GS. MUP-uPA/NOD-SCID mice are currently being bred in our laboratory.

Based on our data with mice, human liver tissue from patients with pre-malignant CLDs that are known to increase HCC risk are expected to contain putative HIC. The frequency of these cells within a given set of samples should positively correlate with published HCC risk factors for the corresponding condition. We also expect that human hepatocytic aggregates will contain cells positive for HIC markers and will be able to give rise to FAH and HCC in immunocompromised mice. We already have ample experience with the protocols required for this work and are adept at performing them. However, should FAH, nodules or tumors fail to form, it is plausible that human hepatocytic aggregates are devoid of HIC. This would be important and we would pursue alternative approaches for isolating human HIC based upon cell surface marker expression (i.e., EpCAM or CD44). Alternatively, human hepatocytic aggregates may contain HIC but fail to form tumors under the current conditions. Should this be the case, we would treat recipient mice with CCl₄ to induce additional chronic liver injury that may create a milieu that better promotes malignant progression. MUP-uPA/NOD-SCID mice may also be poor recipients that reject the human transplants. In such a case, we will replace them with MUP-uPA/Rag2^{-/-}/γ_{c}^{-/-} mice, which are excellent recipients for human grafts⁷⁸.

Statistical Considerations/Analysis:

Experiments will be performed in replicates (n≧3). Outcomes will be compared using 2-sided hypothesis tests at 5% significance. Continuous data will be compared between groups using the Mann-Whitney test. Categorical variables will be compared using the X² or Fisher's exact tests. Paired comparison of continuous data will be performed using the Wilcoxon signed ranks test. Correlations between continuous variables will be tested using the Spearman correlation coefficients. Multivariate analysis of biomarkers as prognosticators will be performed using a Cox regression model.

### Example 10

### e. Develop Methods to Target and Specifically Eliminate Pre-Malignant HIC and Thereby Prevent and/or Treat HCC

The goal of this work is to examine whether antibody-toxin conjugates and liposome-mediated delivery of tumoricidal genes or specific pathway inhibitors can eliminate pre-malignant HIC in the different animal models and prevent HCC development.

In addition to inhibition of signaling pathways needed for HIC formation and maintenance, there are other ways to target these pre-malignant cells and eliminate them prior to HCC development. One approach is to use antibody-drug conjugates (ADC) to specifically deliver cytotoxic drugs. A major problem in HCC treatment is the frequent presence of this cancer in a dysfunctional, diseased liver, thus rendering the patient highly susceptible to drug toxicity. The best way to reduce nonspecific toxicity is to deliver the drug only to cells that need to be destroyed. This can be accomplished through the use of ADC that recognize antigens that are highly expressed on cancer cells relative to normal cells⁷⁹. An effective ADC was prepared by conjugating the cellular metabolite (DM1) of the cytotoxic pro-drug Maytansine to a Her2 antibody^{80,81}. We will therefore couple monoclonal antibodies to CD44 or other HIC specific antigens to DM1 as described^{82,83}. DEN-injected BL6 mice will be treated weekly with either unconjugated antibodies or antibody-DM1 conjugates at a dose of 4 mg/kg starting at 3 months post-DEN injection. After 4 treatment rounds, some of the mice will be sacrificed 1 week after the last treatment and analyzed by IHC for HIC markers and AFP⁺ cells. We will examine liver sections for signs of HIC destruction and death. The remaining mice will be analyzed 8 months after DEN-injection for HCC development as previously described.

A second approach to HIC elimination is specific delivery into these cells of a gene encoding a pro-apoptotic or a cytotoxic molecule or an enzyme that converts a non-toxic pro-drug to a toxic product. Such an enzyme is the Herpes simplex virus thymidine kinase (HSVtk) which converts non-toxic ganciclovir to ganciclovir-monophosphate, which is further converted to highly toxic ganciclovir-triphosphate (G3P) by cellular kinases⁸⁴. G3P is a dGTP analog that prevents DNA replication, thereby affecting only proliferating cells and sparing quiescent cells, such as normal hepatocytes⁸⁵. We will express HSVtk from the AFP promoter/enhancer which is active only in pre-neoplastic and HCC cells^{86,87}, to avoid HSVtk expression in normal hepatocytes. The AFP-HSVtk construct will be delivered to HIC via liposomes, composed of synthetic cationic lipid bilayers which can be complexed with plasmid DNA using established procedures^{88,88}. To target liposomes primarily to HIC, they will also contain a monoclonal antibody to an HIC cell surface marker, for instance CD44. The DNA-immunoliposomes will be produced using established procedures⁸⁹, in collaboration with Sungho Jin at our Bioengineering department who has extensive experience in liposome and nanoparticle production. This approach will combine the efficacy of liposomal delivery with three safety features: delivery of liposomes mainly to CD44⁺ cells, expression of HSVtk only in AFP⁺ cells and production of a cytotoxic molecule that only kills dividing cells. Liposomes will be delivered via an intra-portal vein injection to allow maximal delivery into liver. Mice will be injected with liposomes twice with one week interval starting 3 months after DEN injection and treated with ganciclovir for six days following each liposome injection as described⁸⁶. As a control, we will treat mice with liposomes loaded with empty vector DNA and ganciclovir as above. Mice will be evaluated for presence and death of HIC and HCC formation as above.

We will test several different antibodies against HIC antigens using the well described procedures for DM1 coupling⁸³. The CD44 antibody, and other antibodies to HIC markers, will be tested in targeting liposomes to HIC. To determine the efficiency of HSVtk DNA delivery and expression, we will stain livers of treated mice with antibodies to HSVtk to make sure the viral enzyme is efficiently expressed in the majority of HIC and only in HIC. These experiments will serve as a blueprint for preparation of similar reagents for targeting human HIC.

### Example 11

### Experimental Procedures Used in Examples 12-16

Mice and HCC Induction

MUP-uPA transgenic mice were previously described Weglarz et al (2000). Am J Pathol 157, 1963-1974. Stat3^{Δhep} mice were as previously described (Lee et al., 2002 Immunity 17, 63-72.) All mouse experimental protocols were approved by the UCSD Animal Care Program, following National Institutes of Health guidelines. Histology, gene expression and cell signaling were analyzed as described (Maeda et al., 2005 Cell 121, 977-990; Sakurai et al., 2008 Cancer Cell 14, 156-165). Human HCC specimens were from Department of Internal Medicine, Medical University of Vienna. Immunohistochemical staining of HCC specimens as well as retrospective clinical data collection and analysis were approved by the local ethics committee of the Medical University of Vienna, Austria.

Ikkβ^{f/f} mice (Park et al., 2002) were backcrossed into the C57BL/6 background for at least 6 generations. Ikkβ^{f/f}/Mx1-Cre mice were described (Maeda et al., 2005). To induce HCC, 15 days old littermates were injected with 25 mg/kg DEN (Sigma, St Louis, Mo.). DEN-injected mice were sacrificed either 3 months after DEN injection to be used as hepatocyte donors or maintained for 8 months to monitor HCC development.

Isolation and Transplantation of Primary Hepatocytes

Primary hepatocytes were isolated from DEN-treated mice as described (Leffert et al., 1979). For transplantation, cell preparations whose viability was greater than 80% were used. Three weeks old MUP-uPA transgenic mice received 1.2×10⁵ viable hepatocytes in 30 μl PBS via intra-splenic injection with a 30 G needle (Weglarz et al., 2000). Transplanted mice were sacrificed 5 months later to monitor HCC development. To delete Ikkβ in transplanted hepatocytes, mice were given 1×10⁹ pfu of Adv-GFP or Adv-Cre via the tail vein one month post-transplantation. Alternatively, Ikkβ^{f/f}/Mx1-Cre hepatocytes were transplanted as above and the recipients given 3 injections (250 μg each) of poly(IC) every other day one month after hepatocyte transplantation.

Biochemical and Histological Analyses

RNA extraction and q-PCR were described (Sakurai et al., 2006). Immunoblot analysis, immunohistochemistry, and kinase assays were also described (Maeda et al., 2005). Antibodies used were: anti-IKKβ (Upstate), anti-JNK1/2 (Pharmingen), anti-c-Jun (Santa Cruz), anti-ERK, and anti-phospho-ERK (Cell Signaling), anti-STAT3 (Santa Cruz), anti-phospho-STAT3 (Cell signaling), anti-Albumin (Novus), anti-SHP1 (Santa Cruz), anti-SHP2 (Cell signaling), anti-SOCS3 (Immuno-Biological Laboratories), anti-phospho-p65 (Cell signaling). Anti-AFP antibody was a generous gift from Stewart Sell at the Wadsworth Center and Ordway Research Institute at Albany, N.Y. GFP- and SHP2-expressing adenoviruses were kindly provided by Anton M. Bennett at Yale University School of Medicine (Fornaro et al., 2006). To measure SHP1/2 phosphatase activities, SHP1 and SHP2 were immunoprecipitated from freshly prepared lysates. Dithiothreitol (DTT) and other reducing agents were excluded from all buffers. SHP1/2 phosphatase activities were determined as described (Lee and Esselman, 2002).

Hepatoma Cell Culture and Subcutaneous Inoculation

An HCC-bearing Ikkβ^{f/f} (or Stat3^{f/f}) mouse was anesthetized and the liver was perfused via the portal vein with 20 ml of pre-warmed perfusion buffer (10 mM HEPES and 0.25 mM EGTA in Ca²⁺- and Mg²⁺-free Hank's balanced salt solution, pH=7.4) followed by 20 ml of digestion buffer (10 mM HEPES and 1 mg of liberase blendzyme 3 (Roche Diagnostics, Indianapolis, Ind.) in Hank's BSS with Ca²⁺ and Mg²⁺, pH=7.4, 37° C.). Tumors which are more resistant to digestion than the surrounding liver tissue were dissected, pooled, and washed in ice-cold PBS. In case of incomplete digestion, tumors were minced and incubated with the above digestion buffer at 37° C. with gentle stirring for up to 1 hr. Cells were filtered through a 100 μm cell strainer and collected by centrifugation at 50×g for 30 sec. Cells were washed once with 10 ml PBS and plated in growth medium: 20% heat inactivated FBS, 0.01 g/L insulin, 0.01 g/L hydrocortisone hemisuccinate, 1% penicillin-streptomycin, 0.25 mg/L amphotericin B, 1% L-glutamine, 1 mM phenobarbital and 20 μg/L EGF in DMEM. When confluent (2-3 weeks later), cells were differentially trypsinized and passaged. Cell strains obtained in this way from each mouse were designated as DEN-induced HCC 1 (dih1) and so on. Cells were frozen at early passages. Cells used in our experiments did not exceed 8 passages in culture.

To generate hepatospheres, dih cells were enzymatically and mechanically dispersed into a single cell suspension and plated onto a Petri dish in the above described growth medium but without serum. Hepatospheres were counted 10 days after plating and dispersed for further passage.

To delete IKKβ (or STAT3), dih cells were infected with Adv-GFP or Adv-Cre at an MOI=20 overnight. The cells were cultured for at least 5 additional days to ensure complete deletion. For subcutaneous inoculation, 2.5×10⁶ viable cells in 100 μl PBS were mixed with 50 μl of Matrigel (BD, San Jose, Calif.) and injected to the back flanks of 8 weeks old C57BL/6 male mice. Starting on the next day, mice were given various treatments as described. MLN120B was provided by Millennium. AG490 and BHA were from Sigma and S3I-201 was from Santa Cruz.

shRNA Expression

For shRNA-induced gene silencing, we used the pLSLP lentiviral vector (Budanov and Karin, 2008). 5′-AGAGAAGGTAGGACATTCT-3′ and 5′-AGAGAAGGTAGGACATTCT-3′ were used for Jnk1/2 shRNA and for control shRNA, respectively. The sequences used to target mouse Stat3 and Jak2 were 5′-GGTATAACATGCTGACCAA-3′ and 5′-GAGAATAGCTAAGGAGAAA-3′, respectively.

Statistical Analysis

Data are presented as means±s.d. Differences in means were analyzed by Student's t test and one-way ANOVA. Tumor incidence (%) was analyzed by chi-square analysis. p values <0.05 were considered significant.

Human HCC Specimens and their Analysis

Human HCC specimens were from Department of Internal Medicine, Division of Gastroenterology/Hepatology Medical University, Vienna. HCC samples were obtained during liver transplantation from a total of 52 patients (49 males, 3 females) who had no prior therapy before surgery (Sieghart et al., 2006). None of the patients was diagnosed with regional lymph node metastasis and only one patient had distal metastasis at the time of liver transplantation. Sections prepared from paraffin-embedded blocks were stained with either phospho-STAT3 antibody (Cell Signaling) or phospho-p65 antibody (Cell Signaling) at a dilution of 1:50. Positive nuclear staining was scored. (***)=large area staining; (**)=staining of multiple smaller areas; (*)=staining of scattered few positive cells.

### Example 12

### A Transplant System for Studying Progression/Malignant Conversion of Initiated Hepatocytes

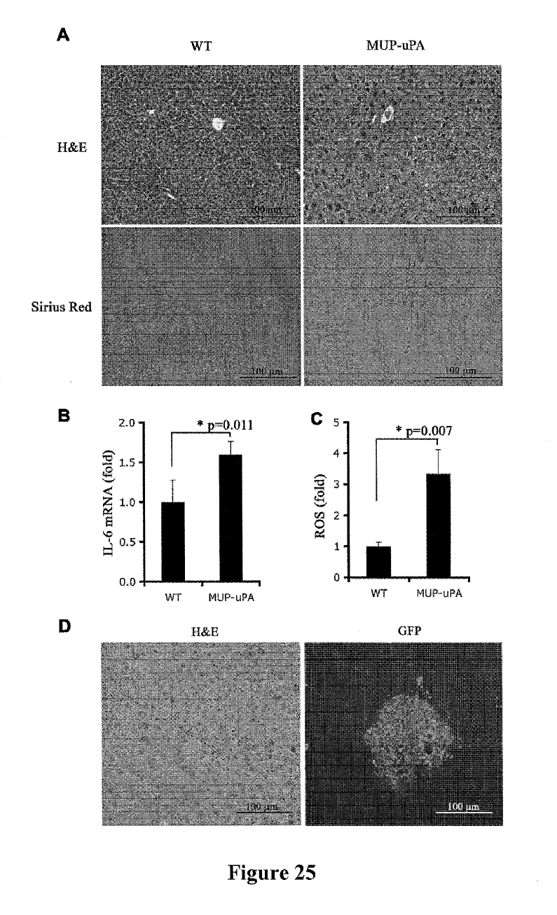

To dissociate initiation and early tumor promotion from HCC progression and malignant conversion, we established an experimental system for studying late events that affect hepatocarcinogenesis. We treated C57BL/6 mice with DEN at 2 weeks of age and waited for 3 months to allow hepatocyte initiation and clonal expansion. At that point, hepatocytes were isolated and transplanted via splenic injection into livers of MUP-uPA transgenic mice (FIG. 18A). The latter over-express urokinase-type plasminogen activator (uPA) in their hepatocytes and are therefore subjected to low grade but continuous liver damage and regeneration, making them ideal recipients for exogenous hepatocytes (Weglarz et al., 2000). Furthermore, MUP-uPA mice develop mild liver fibrosis but no HCC by 8 months of age (FIG. 25A). Their livers also exhibit elevated expression of IL-6 mRNA (FIG. 25B) and enhanced ROS accumulation (FIG. 25C). All of these changes resemble the microenvironment within which human liver cancer forms. Within a month, transplanted hepatocytes marked with green fluorescent protein (GFP) formed small islands in the recipient's liver but otherwise were barely distinguishable from host hepatocytes (FIG. 25D). By 5 months after transplantation, male MUP-uPA mice receiving hepatocytes from DEN-initiated males developed multiple tumor nodules that were absent in mice receiving hepatocytes from vehicle-injected mice (FIG. 18B) or in untransplanted MUP-uPA mice. The tumors, which exhibited a typical trabecular HCC structure and expressed albumin and elevated amounts of the HCC marker α-fetoprotein (AFP; FIG. 18B,C), are likely to be derived from the transplanted DEN-initiated cells. The latter failed to grow in normal C57BL/6 mice, suggesting that the MUP-uPA liver microenvironment is conducive and essential for conversion of initiated hepatocytes into HCC.

To further characterize this system and determine its physiological relevance, we examined whether the host gender affects HCC formation by transplanted hepatocytes. We injected male and female mice with DEN and transplanted their hepatocytes into MUP-uPA hosts of either gender. Five months later, male recipients of male hepatocytes exhibited at least one HCC per liver, whereas less than 50% of female recipients of male hepatocytes bore tumors (FIG. 18D). Furthermore, tumor multiplicity was 5-times higher in male hosts (FIG. 18E). Even more striking results were obtained with transplanted female hepatocytes (FIG. 18F, G). Although fewer tumors were seen in this case, consistent with the gender bias in HCC induction (Naugler et al., 2007), male hosts of initiated female hepatocytes exhibited 4-fold higher tumor incidence and nearly 20-times higher tumor multiplicity than female hosts of female hepatocytes (FIG. 18F, G). Since MUP-uPA expression is similar between males and females (Weglarz et al., 2000), these data demonstrate that gender plays a critical role not only in HCC initiation and early promotion but also in tumor progression. Removing initiated hepatocytes from the female microenvironment where they hardly progress into HCC to a male microenvironment results in a substantial enhancement of HCC development. These findings support the physiological relevance of the transplant system.

### Example 13

### Deletion of Ikkβ in Initiated Hepatocytes Enhances Tumorigenic Potential

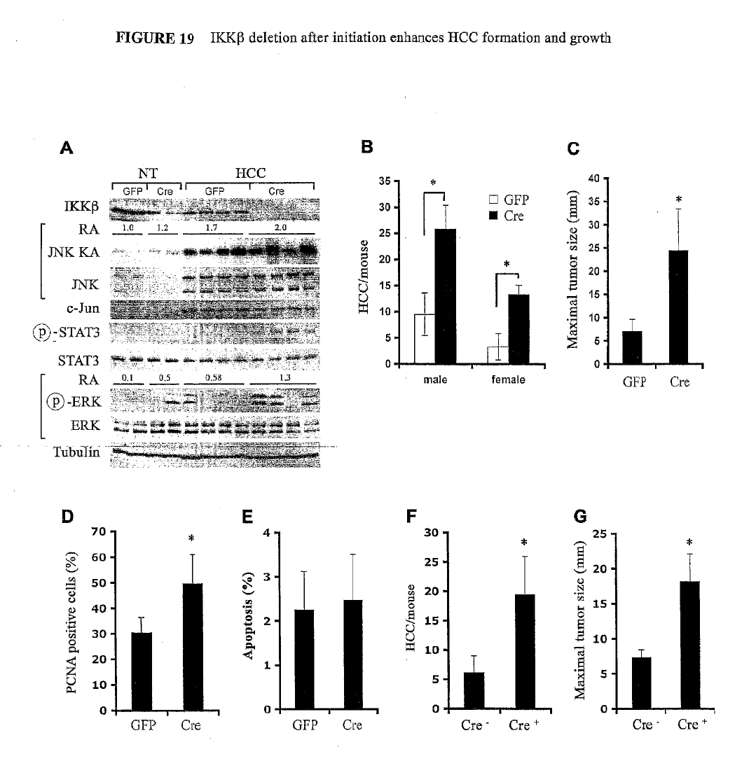

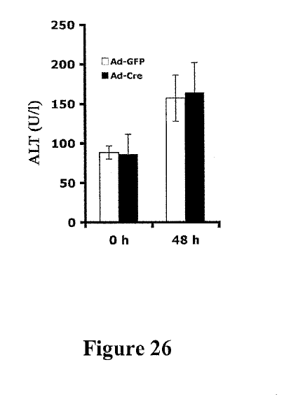

Next we examined whether the IKKβ-NF-κB pathway, which inhibits DEN-induced (Maeda et al., 2005) and spontaneous (Luedde et al., 2007) HCC development, most likely by suppressing death-driven compensatory proliferation that occurs during early tumor promotion (Karin, 2006), also affects progression. To this end, we gave Ikkβ^{f/f} male mice, which express normal amounts of IKKβ, DEN and transferred their initiated hepatocytes into MUP-uPA mice. After one month, male and female hosts were injected with GFP- or Cre-expressing adenoviruses (Adv) to delete Ikkβ in transplanted hepatocytes. Adv infection caused mild liver injury, indicated by a small elevation in circulating ALT (FIG. 26). Administration of Adv-Cre induced efficient IKKβ deletion (FIG. 19A) and resulted in a 3-4-fold increase in tumor multiplicity and size in both male and female recipients relative to Adv-GFP infection (FIG. 19B, C).

In addition to complete IKKβ deletion, Adv-Cre administration increased STAT3 and ERK phosphorylation in HCCs relative to Adv-GFP administration (FIG. 19A). However, JNK and c-Jun expression and JNK kinase activity, which were elevated in HCCs relative to non-tumor liver tissue, did not show large differences between IKKβ-expressing and non-expressing HCCs. Ikkβ-deleted HCCs contained more proliferating (PCNA-positive) cells than IKKβ-expressing tumors (FIG. 19D), but the rate of HCC apoptosis was not affected by the IKKβ status (FIG. 19E).

As an alternative approach to delete Ikkβ after tumor initiation, we used DEN-initiated Ikkβ^{f/f}/Mx1-Cre mice as hepatocyte donors. These mice express Cre recombinase from the interferon (IFN)-inducible Mx1 promoter (Kuhn et al., 1995), such that administration of the IFN-inducer poly(IC) results in efficient Ikkβ deletion in liver (Maeda et al., 2005). Using this experimental set-up, we deleted Ikkβ one month after transplantation. This resulted in a large increase in HCC multiplicity and size in hosts receiving initiated hepatocytes from Ikkβ^{f/f}/Mx1-Cre donors relative to hosts transplanted with Ikkβ^{f/f} hepatocytes (FIG. 19F,G). These results clearly demonstrate that in addition to enhancing tumor initiation and/or early promotion, deletion of Ikkβ in initiated hepatocytes augments and/or accelerates HCC progression.

### Example 14

### Ikkβ Deletion Enhances Hepatosphere Formation and Tumorigenic Potential

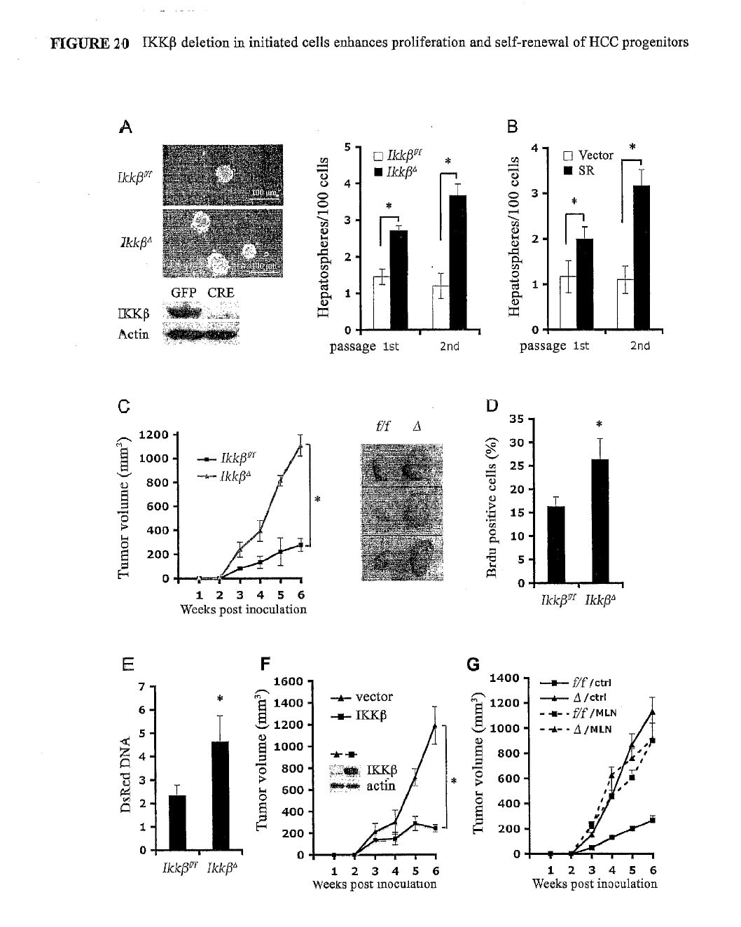

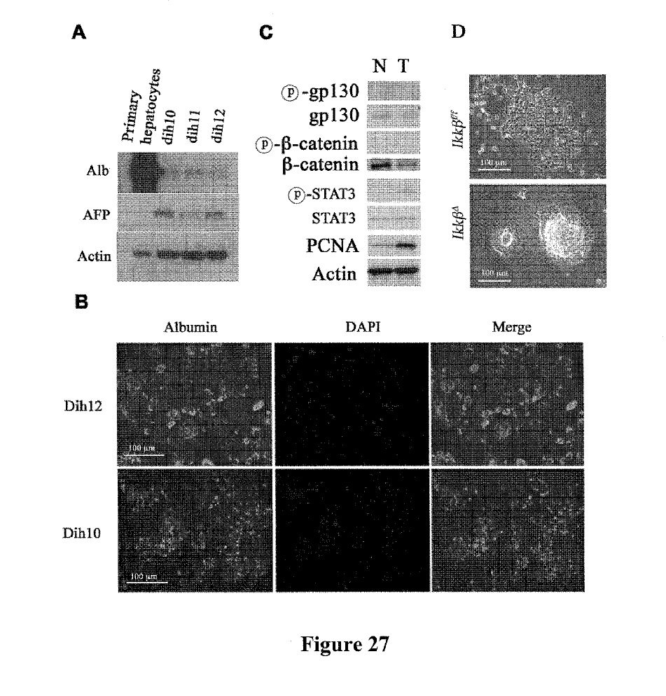

To further examine cell autonomous effects of IKKβ in malignant hepatocytes, we cultured DEN-induced HCCs from Ikkβ^{f/f} mice. Initially, HCC cells failed to proliferate and gradually died in standard hepatocyte culture medium. Addition of phenobarbital, a liver tumor promoter (Kaufmann et al., 1988), and EGF overcame this problem and allowed the derivation of several cell strains from DEN-induced HCCs (dih). Three of the strains (dih10-12) expressed both albumin and AFP, consistent with being derived from AFP-expressing HCCs (FIG. 27A). All dih cells were albumin positive, suggesting little contamination, if any, with non-parenchymal cells (FIG. 27B). These cells showed increased PCNA expression and enhanced STAT3 phosphorylation relative to primary hepatocytes, but did not exhibit an obvious increase in gp130 or β-catenin phosphorylation under standard culture conditions (FIG. 27C). Infection of dih cells with Adv-Cre resulted in nearly complete Ikkβ deletion (Ikkβ^{Δ}) (FIG. 20A). Ikkβ^{Δ} dih cells grew in multi-layers and formed spheroids even under non-confluent conditions, while Ikkβ^{f/f} dih cells mainly grew as monolayers (FIG. 27D). When plated onto Petri dishes without serum, Ikkβ^{f/f} dih cells formed a few floating spheroids (hepatospheres) that could be passaged in culture to yield secondary hepatospheres (FIG. 20A). Interestingly, Ikkβ^{Δ} dih cells formed twice as many primary hepatospheres and 3-fold more secondary hepatospheres than Ikkβ^{f/f} cells. Transduction of Ikkβ^{f/f} dih cells with an IκB super-repressor lentivirus (Boehm et al., 2007) also enhanced hepatosphere formation (FIG. 20B), suggesting that IKKβ inhibits hepatosphere formation via NF-κB.

In mammary cancer, spheroid-forming cells were suggested to be tumor progenitors (Al-Hall and Clarke, 2004). To test whether Ikkβ deletion in dih cells enhances tumorigenic potential, we subcutaneously implanted Ikkβ^{f/f} and Ikkβ^{Δ} dih10 cells into C57BL/6 mice. Ikkβ^{Δ} cells grew faster than Ikkβ^{f/f} cells and after 6 weeks formed tumors that were 4-times larger than those formed by Ikkβ^{f/f} cells (FIG. 20C) and had higher proliferative index (FIG. 20D). A similar difference in proliferative potential between Ikkβ^{f/f} and Ikkβ^{Δ} dih cells was seen when the cells were grown in the liver microenvironment: dsRed-labeled Ikkβ^{Δ} dih12 cells formed faster growing HCCs in MUP-uPA livers than Ikkβ^{f/f} dih12 cells (FIG. 20E). Conversely, retroviral-mediated reconstitution of IKKβ in Ikkβ^{Δ} dih10 cells suppressed tumorigenic growth (FIG. 20F). Thus, the effect of Ikkβ deletion on hepatoma growth is reversible. Enhanced tumorigenic growth of subcutaneously inoculated dih10 cells was also seen upon treatment of tumor-bearing mice with the specific IKKβ inhibitor MLN120B (Wen et al., 2006). MLN120B enhanced tumorigenic growth of Ikkβ^{f/f} dih cells and not Ikkβ^{Δ} cells and its effect was equivalent to that of Ikkβ deletion (FIG. 20G). These data demonstrate that IKKβ is an inhibitor of HCC growth and progression and suggest that its effect is direct and not due to irreversible genetic alterations.

### Example 15

### STAT3 Activity is Elevated in the Absence of IKKβ Due to ROS-Mediated SHP1/2 Inactivation

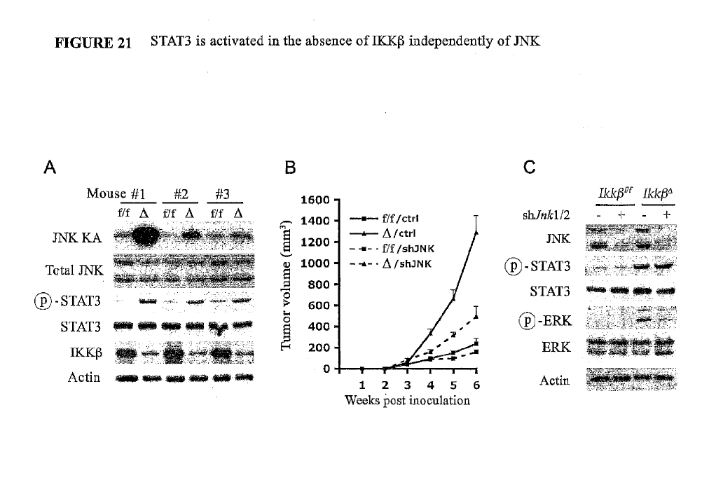

To determine how loss of IKKβ accelerates tumor growth and progression, we examined its effect on signaling pathways that affect hepatocyte proliferation. Subcutaneous tumors formed by Ikkβ^{Δ} dih cells exhibited a tendency towards higher JNK activity, but the effect was variable (FIG. 21A). A more consistent change was increased STAT3 phosphorylation in Ikkβ^{Δ} dih tumors. Despite the variable effect of IKKβ on JNK activity in HCCs and subcutaneous tumors, silencing of JNK1/2 expression in dih10 cells suppressed their tumorigenic growth (FIG. 21B) and the inhibitory effect was greater in Ikkβ^{Δ} cells. Curiously, silencing of JNK1/2 expression decreased ERK phosphorylation in Ikkβ^{Δ} tumors, but had no effect on STAT3 phosphorylation (FIG. 21C).

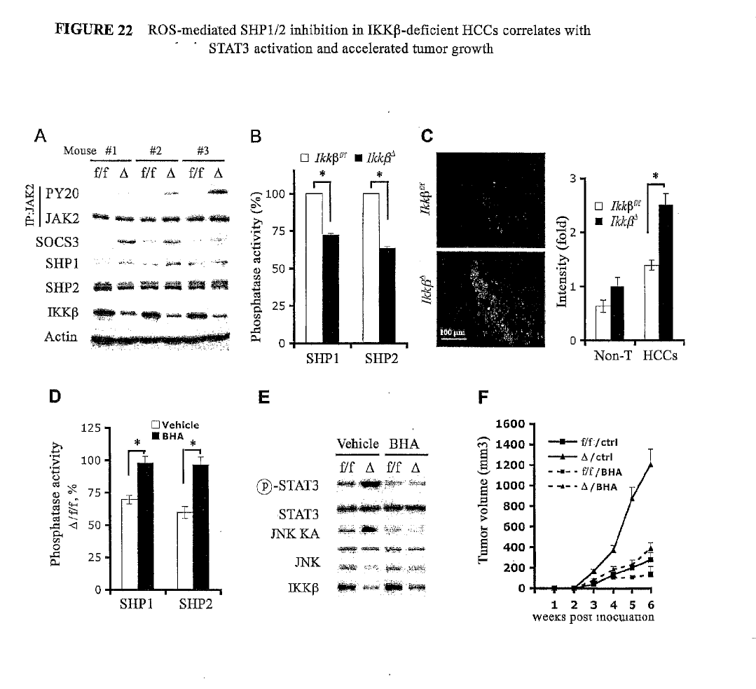

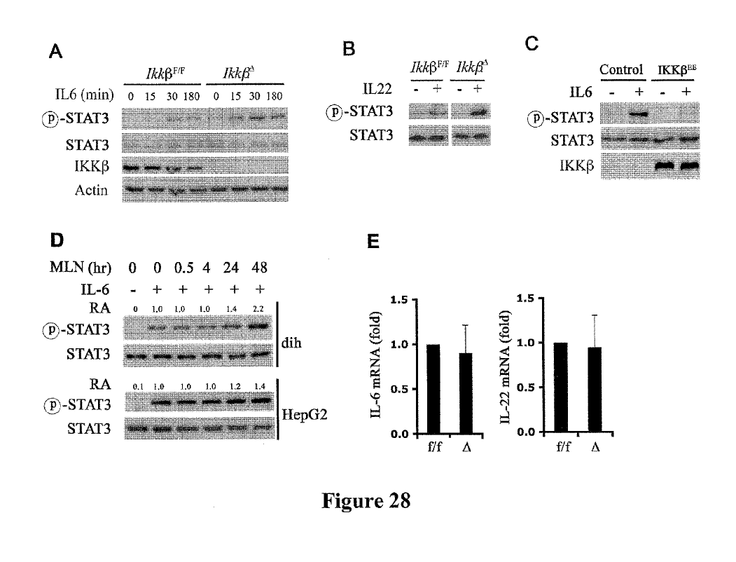

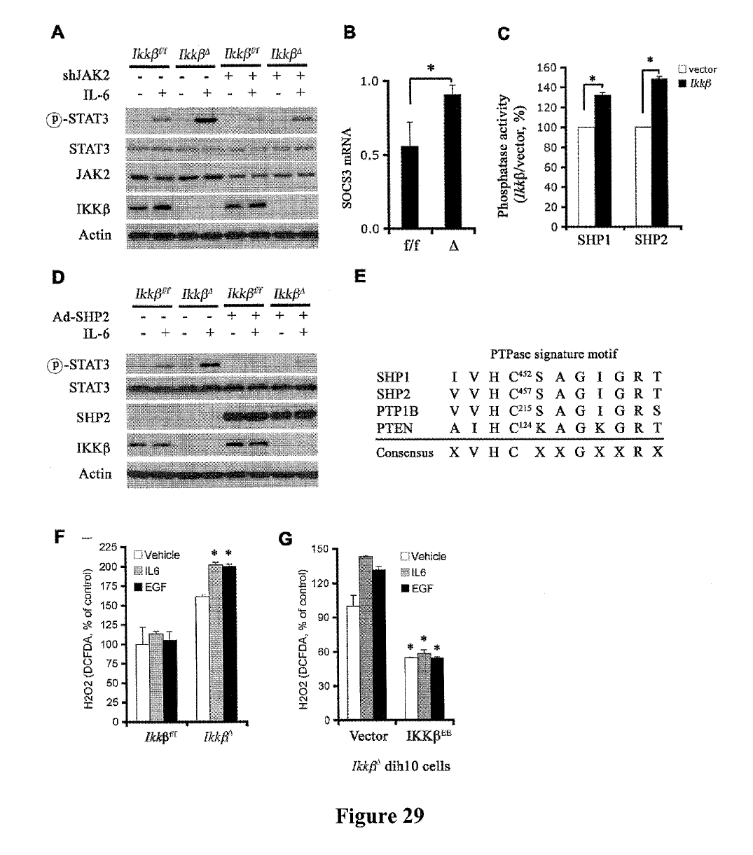

We examined the cause of elevated STAT3 activity in Ikkβ^{Δ} dih cells. In vitro, both IL-6 and IL-22, which are major STAT3 activators in liver (Naugler et al., 2007; Zenewicz et al., 2007), led to higher STAT3 activity in Ikkβ^{Δ} dih cells than in Ikkβ^{f/f} dih cells (FIG. 28A, B). Conversely, expression of constitutively active IKKβ (IKKβ^{EE}) in Ikkβ^{Δ} dih cells inhibited IL-6 induced STAT3 activation (FIG. 28C). The IKKβ inhibitor MLN120B also enhanced IL-6 induced STAT3 activation in dih cells and a human liver cancer cell line (FIG. 28D). Enhancement of STAT3 activation required a 24-48 hr pre-incubation with MLN120B, suggesting that IKKβ regulates STAT3 indirectly. No obvious differences in IL-6 or IL-22 expression were detected between IKKβ-expressing and non-expressing HCCs (FIG. 28E). Given these results, we considered that increased STAT3 activity in Ikkβ^{Δ} dih cells is due to a cell intrinsic effect on the STAT3 signaling pathway. In support of this, phosphorylation of JAK2, a Janus kinase involved in IL-6-mediated STAT3 activation (Kamimura et al., 2003), was enhanced in tumors formed by Ikkβ^{Δ} dih cells relative to Ikkβ^{f/f} dih tumors (FIG. 22A), suggesting that elevated STAT3 activation in Ikkβ^{Δ} dih cells is the consequence of enhanced JAK2 activity. Indeed, inhibition of JAK2 expression by shRNA reduced IL-6 induced STAT3 activation in both Ikkβ^{f/f} and Ikkβ^{Δ} dih cells (FIG. 29A). We also examined other regulators of the JAK2-STAT3 pathway. Expression of SOCS3, a critical feedback inhibitor of cytokine signaling and a STAT3 target gene (Auernhammer et al., 1999; Brender et al., 2001), whose ablation enhances HCC development (Ogata et al., 2006), was elevated in IKKβ-deficient tumors (FIG. 22A). SOCS3 upregulation is likely to be transcriptional because SOCS3 mRNA was also higher in the absence of IKKβ (FIG. 29B). Thus, enhanced JAK2 activation cannot be due to diminished SOCS3 expression as previously found in the hypothalamus (Zhang et al., 2008).

Other negative regulators of JAK2-STAT3 signaling include the SH2-containing phosphatases, SHP1 and SHP2 (Valentino and Pierre, 2006). Tumors derived from dih cells expressed higher amounts of SHP2 than SHP1, but these were not significantly affected by IKKβ (FIG. 22A). However, both SHP1 and SHP2 phosphatase activities were significantly lower in tumors formed by Ikkβ^{Δ} dih cells relative to Ikkβ^{f/f} dih tumors (FIG. 22B). SHP1 and SHP2 activities in Ikkβ^{Δ} tumors were restored upon reconstitution with IKKβ (FIG. 29C). To validate a role for SHP1/2 in regulation of STAT3, we overexpressed SHP2, the more abundant of the two phosphatases in HCC cells, and this resulted in a strong inhibition of IL-6 induced STAT3 phosphorylation in both Ikkβ^{f/f} and Ikkβ^{Δ} dih cells (FIG. 29D). These results strongly suggest that reduced SHP1/2 activities in IKKβ-deficient HCCs are responsible for the elevated STAT3 activation.

SHP1/2 are members of the protein tyrosine phosphatase (PTP) family (FIG. 29E), whose catalytic cysteine is highly susceptible to oxidation (Meng et al., 2002; Salmeen et al., 2003). Several PTPs are subject to reversible inactivation in response to growth factor (Meng et al., 2002) or cytokine induced ROS and this inactivation is potentiated in the absence of NF-κB (Kamata et al., 2005). We therefore examined ROS accumulation in HCCs and in dih cells. Staining with the ROS (superoxide) indicator dihydroethydine (DHE) revealed more ROS accumulation in HCCs relative to surrounding tissue and IKKβ-deficient HCCs stained stronger than IKKβ-expressing HCCs (FIG. 22C). Cultured Ikkβ^{Δ} dih cells also accumulated more ROS both under basal culture conditions and in response to IL-6 or EGF than Ikkβ^{f/f} dih cells and this was reversed by expression of constitutively active IKKβ^{EE} (FIG. 29F,G). To examine the role of ROS in reduced SHP1/2 activities and STAT3 upregulation in IKKβ-deficient HCCs, we fed tumor-bearing mice with BHA, a potent anti-oxidant (Maeda et al., 2005). BHA feeding completely restored SHP1/2 phosphatase activities and reduced JNK activity and STAT3 phosphorylation in tumors formed by Ikkβ^{f/f} dih cells to levels comparable to those in Ikkβ^{f/f} tumors (FIG. 22D, E). Importantly, BHA consumption reduced Ikkβ^{Δ} tumor growth to a level that was similar to that of Ikkβ^{f/f} tumors in untreated mice (FIG. 22F). Collectively, these data suggest that ROS accumulation in IKKβ-deficient HCCs is responsible for reduced PTP activity, JNK and STAT3 activation, as well as accelerated tumor growth.

### Example 16

### STAT3 Activity is Required for HCC Formation and Growth

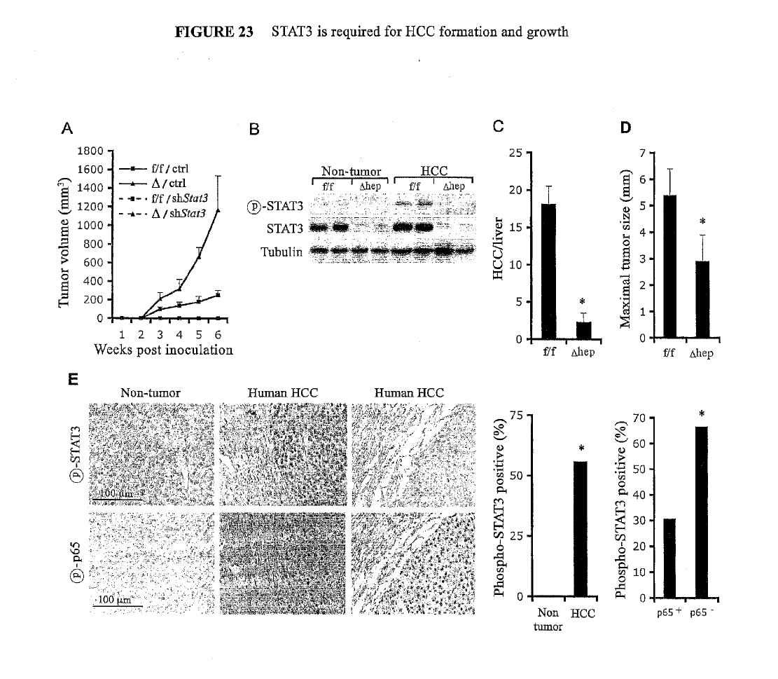

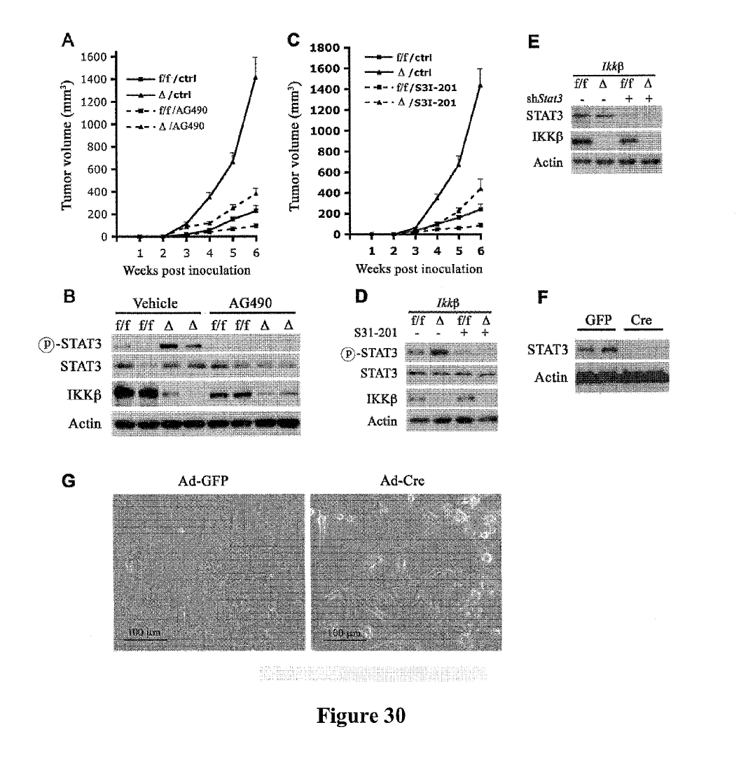

To examine the contribution of activated STAT3 to the enhanced tumorigenic potential of Ikkβ^{Δ} dih cells, we treated tumor-bearing mice with AG490, an inhibitor of STAT3 phosphorylation (Eriksen et al., 2001). AG490 inhibited the growth of Ikkβ^{Δ} subcutaneous tumors and had a more modest effect on Ikkβ^{f/f} tumorigenic growth (FIG. 30A). Immunoblot analysis verified that AG490 inhibited STAT3 phosphorylation regardless of IKKβ status (FIG. 30B). A similar effect on tumor growth was observed with another STAT3 inhibitor, S3I-201 (FIG. 30C), which inhibits STAT3 activation through binding to its SH2 domain (Siddiquee et al., 2007). S3I-201 also inhibited STAT3 phosphorylation (FIG. 30D). To more specifically address the role of STAT3 we silenced its expression in dih cells via lentiviral expression of a STAT3-specific shRNA (FIG. 30E). In sharp contrast to cells transduced with a control lentivirus encoding scrambled shRNA which formed subcutaneous tumors, dih cells transduced with the STAT3 shRNA failed to grow into subcutaneous tumors regardless of their IKKβ status (FIG. 23A).

To further examine the role of STAT3 in HCC development, we administered DEN to 2 weeks old hepatocyte-specific STAT3-deficient mice (Stat3^{f/f}/Alb-Cre mice=Stat3^{Δhep}) (FIG. 23B). Stat3^{Δhep} mice were markedly resistant to DEN-induced HCC development with more than 6-fold reduction in HCC multiplicity relative to State mice (FIG. 23C). Tumors in Stat3^{Δhep} mice, which retained their STAT3 deficiency (FIG. 23B), were also significantly smaller than HCCs in Stat3^{f/f} mice (FIG. 23D). We derived Stat3^{f/f} dih cells from DEN-induced HCCs of Stat3^{f/f} mice, but deletion of STAT3 from these cells by Ad-Cre infection resulted in inhibition of cell growth and induction of cell death (FIG. 30F,G). These data strongly suggest that STAT3 is required for mouse HCC development, growth and survival.

We also examined the status of STAT3 activation in more than 50 different human HCC specimens and found that nearly 60% of them exhibited activated nuclear STAT3, which could not be detected in non-tumor liver tissue from the same patient (FIG. 23E). As observed previously (Calvisi et al., 2006), STAT3 activation was more pronounced in the more aggressive tumors (FIG. 31 (Table S1)). We also found that 25% (13/52) of human HCCs were positive for phospho-p65/RelA, an indicator of NF-κB activation. However, phospho-p65 positive HCCs were often negative for activated STAT3. Only 30.8% of phospho-p65 positive HCCs were positive for activated STAT3, whereas 66.7% of phospho-p65 negative HCCs exhibited activated STAT3 (FIG. 23E). These data show that a major sub-fraction of human HCCs exhibit an inverse relationship between NF-κB and STAT3.

### REFERENCES CITED IN THE ABOVE “DESCRIPTION OF THE INVENTION” SECTIONS A AND H-J

1. Visvader, J E and Lindeman, G J. Cancer stem cells in solid tumours: accumulating evidence and unresolved questions. Nat Rev Cancer, 2008. 8(10): p. 755-68.
2. Reya, T, Morrison, S J, Clarke, M F, and Weissman, I L. Stem cells, cancer, and cancer stem cells. Nature, 2001. 414(6859): p. 105-11.
3. Lapidot, T, Sirard, C, Vormoor, J, Murdoch, B, Hoang, T, Caceres-Cortes, J, Minden, M, Paterson, B, Caligiuri, M A, and Dick, J E. A cell initiating human acute myeloid leukaemia after transplantation into SCID mice. Nature, 1994. 367(6464): p. 645-8.
4. Bonnet, D and Dick, J E. Human acute myeloid leukemia is organized as a hierarchy that originates from a primitive hematopoietic cell. Nat Med, 1997. 3(7): p. 730-7.
5. Al-Hajj, M, Wicha, M S, Benito-Hernandez, A, Morrison, S J, and Clarke, M F. Prospective identification of tumorigenic breast cancer cells. Proc Natl Acad Sci USA, 2003. 100(7): p. 3983-8.
6. O'Brien, C A, Pollett, A, Gallinger, S, and Dick, J E. A human colon cancer cell capable of initiating tumour growth in immunodeficient mice. Nature, 2007. 445(7123): p. 106-10.
7. Schatton, T, Murphy, G F, Frank, N Y, Yamaura, K, Waaga-Gasser, A M, Gasser, M, Zhan, Q, Jordan, S, Duncan, L M, Weishaupt, C, Fuhlbrigge, R C, Kupper, T S, Sayegh, M H, and Frank, M H. Identification of cells initiating human melanomas. Nature, 2008. 451(7176): p. 345-9.
8. Yang, Z F, Ho, D W, Ng, M N, Lau, C K, Yu, W C, Ngai, P, Chu, P W, Lain, C T, Poon, R T, and Fan, S T. Significance of CD90+ cancer stem cells in human liver cancer. Cancer Cell, 2008. 13(2): p. 153-66.
9. Al-Hajj, M, Becker, M W, Wicha, M, Weissman, I, and Clarke, M F. Therapeutic implications of cancer stem cells. Curr Opin Genet Dev, 2004. 14(1): p. 43-7.
10. Gumley, T P, McKenzie, I F, and Sandrin, M S. Tissue expression, structure and function of the murine Ly-6 family of molecules. Immunol Cell Biol, 1995. 73(4): p. 277-96.
11. Marhaba, R, Klingbeil, P, Nuebel, T, Nazarenko, I, Buechler, M W, and Zoeller, M. CD44 and EpCAM: cancer-initiating cell markers. Curr Mol Med, 2008. 8(8): p. 784-804.
12. St John, T, Meyer, J, Idzerda, R, and Gallatin, W M. Expression of CD44 confers a new adhesive phenotype on transfected cells. Cell, 1990. 60(1): p. 45-52.
13. Aruffo, A, Stamenkovic, I, Melnick, M, Underhill, C B, and Seed, B. CD44 is the principal cell surface receptor for hyaluronate. Cell, 1990. 61(7): p. 1303-13.
14. Lapidot, T, Sirard, C, Vormoor, J, Murdoch, B, Hoang, T, Caceres-Cortes, J, Minden, M, Paterson, B, Caligiuri, M A, and Dick, J E. A cell initiating human acute myeloid leukaemia after transplantation into SCID mice. Nature, 1994. 367(6464): p. 645-8.
15. Bonnet, D and Dick, J E Human acute myeloid leukemia is organized as a hierarchy that originates from a primitive hematopoietic cell. Nat Med, 1997. 3(7): p. 730-7.
16. Al-Hajj, M, Wicha, M S, Benito-Hernandez, A, Morrison, S J, and Clarke, M F. Prospective identification of tumorigenic breast cancer cells. Proc Natl Acad Sci USA, 2003. 1OO (7): p. 3983-8.
17. O'Brien, C A, Pollett, A, Gallinger, S, and Dick, J E. A human colon cancer cell capable of initiating tumour growth in immunodeficient mice. Nature, 2007. 445(7123): p. 106-10.
18. Schatton, T, Murphy, G F, Frank, N Y, Yamaura, K, Waaga-Gasser, A M, Gasser, M, Zhan, Q, Jordan, S, Duncan, L M, Weishaupt, C, Fuhlbrigge, R C, Kupper, T S, Sayegh, M H, and Frank, M H. Identification of cells initiating human melanomas. Nature, 2008. 451(7176): p. 345-9.
19. Yang, Z F, Ho, D W, Ng, M N, Lau, C K, Yu, W C, Ngai, P, Chu, P W, Lam, C T, Poon, R T, and Fan, S T. Significance of CD90+ cancer stem cells in human liver cancer. Cancer Cell, 2008. 13(2): p. 153-66.

### REFERENCES CITED IN THE ABOVE “DESCRIPTION OF THE INVENTION” SECTIONS K-M

1. Dvorak H F. Tumors: wounds that do not heal. Similarities between tumor stroma generation and wound healing. N Engl J Med 1986; 315:1650-9.
2. Schafer M, Werner S. Cancer as an overhealing wound: an old hypothesis revisited. Nat Rev Mol Cell Biol 2008; 9:628-38.
3. Coussens L M, Werb Z. Inflammation and cancer. Nature 2002; 420:860-7.
4. Ernst P B, Gold B D. The disease spectrum of Helicobacter pylori: the immunopathogenesis of gastroduodenal ulcer and gastric cancer. Annu Rev Microbiol 2000; 54:615-40.
5. Seeff L B. Introduction: The burden of hepatocellular carcinoma. Gastroenterology 2004; 127:S1-4.
6. Balkwill F, Mantovani A. Inflammation and cancer: back to Virchow? Lancet 2001; 357:539-45.
7. Parkin D M, Bray F I, Devesa S S. Cancer burden in the year 2000. The global picture. Eur J Cancer 2001; 37 Suppl 8:S4-66.
8. Bosch F X, Ribes J, Diaz M, Cleries R. Primary liver cancer: worldwide incidence and trends. Gastroenterology 2004; 127:S5-S16.
9. Tong M J, el-Farra N S, Reikes A R, Co R L. Clinical outcomes after transfusion-associated hepatitis C. N Engl J Med 1995; 332:1463-6.
10. Nakamoto Y, Kaneko S. Mechanisms of viral hepatitis induced liver injury. Curr Mol Med 2003; 3:537-44.
11. Herzer K, Sprinzl M F, Galle P R. Hepatitis viruses: live and let die. Liver Int 2007; 27:293-301.
12. Block T M, Mehta A S, Fimmel C J, Jordan R. Molecular viral oncology of hepatocellular carcinoma. Oncogene 2003; 22:5093-107.
13. Sen R, Baltimore D. Multiple nuclear factors interact with the immunoglobulin enhancer sequences. Cell 1986; 46:705-16.
14. Barnes P J, Karin M. Nuclear factor-kappaB: a pivotal transcription factor in chronic inflammatory diseases. N Engl J Med 1997; 336:1066-71.
15. Ghosh S, Karin M. Missing pieces in the NF-kappaB puzzle. Cell 2002; 109 Suppl: S81-96.
16. Vallabhapurapu S, Karin M. Regulation and function of NF-kappaB transcription factors in the immune system. Annu Rev Immunol 2009; 27:693-733.
17. Gilmore T D. The Rel/NF-kappa B/I kappa B signal transduction pathway and cancer. Cancer Treat Res 2003; 115:241-65.
18. Karin M, Cao Y, Greten F R, Li Z W. NF-kappaB in cancer: from innocent bystander to major culprit. Nat Rev Cancer 2002; 2:301-10.
19. Franzoso G, Bours V, Park S, Tomita-Yamaguchi M, Kelly K, Siebenlist U. The candidate oncoprotein Bcl-3 is an antagonist of p50/NF-kappa B-mediated inhibition. Nature 1992; 359:339-42.
20. Bours V, Franzoso G, Azarenko V, et al. The oncoprotein Bcl-3 directly transactivates through kappa B motifs via association with DNA-binding p50B homodimers. Cell 1993; 72:729-39.
21. Annunziata C M, Davis R E, Demchenko Y, et al. Frequent engagement of the classical and alternative NF-kappaB pathways by diverse genetic abnormalities in multiple myeloma. Cancer Cell 2007; 12:115-30.
22. Keats J J, Fonseca R, Chesi M, et al. Promiscuous mutations activate the noncanonical NF-kappaB pathway in multiple myeloma. Cancer Cell 2007; 12:131-44.
23. Beg A A, Sha W C, Bronson R T, Ghosh S, Baltimore D. Embryonic lethality and liver degeneration in mice lacking the RelA component of NF-kappa B. Nature 1995; 376:167-70.
24. Doi T S, Marino M W, Takahashi T, et al. Absence of tumor necrosis factor rescues RelA-deficient mice from embryonic lethality. Proc Natl Acad Sci USA 1999; 96:2994-9.
25. Rosenfeld M E, Prichard L, Shiojiri N, Fausto N. Prevention of hepatic apoptosis and embryonic lethality in RelA/TNFR-1 double knockout mice. Am J Pathol 2000; 156:997-1007.
26. Li Q, Van Antwerp D, Mercurio F, Lee K F, Verma I M. Severe liver degeneration in mice lacking the IkappaB kinase 2 gene. Science 1999; 284:321-5.
27. Tanaka M, Fuentes M E, Yamaguchi K, et al. Embryonic lethality, liver degeneration, and impaired NF-kappa B activation in IKK-beta-deficient mice. Immunity 1999; 10:421-9.
28. Rudolph D, Yeh W C, Wakeham A, et al. Severe liver degeneration and lack of NF-kappaB activation in NEMO/IKKgamma-deficient mice. Genes Dev 2000; 14:854-62.
29. Makris C, Godfrey V L, Krahn-Senftleben G, et al. Female mice heterozygous for IKK gamma/NEMO deficiencies develop a dermatopathy similar to the human X-linked disorder incontinentia pigmenti. Mol Cell 2000; 5:969-79.
30. Luedde T, Assmus U, Wustefeld T, et al. Deletion of IKK2 in hepatocytes does not sensitize these cells to TNF-induced apoptosis but protects from ischemia/reperfusion injury. J Clin Invest 2005; 115:849-59.
31. Maeda S, Chang L, Li Z W, Luo J L, Leffert H, Karin M. IKKbeta is required for prevention of apoptosis mediated by cell-bound but not by circulating TNFalpha. Immunity 2003; 19:725-37.
32. Luedde T, Heinrichsdorff J, de Lorenzi R, De Vos R, Roskams T, Pasparakis M. IKK1 and IKK2 cooperate to maintain bile duct integrity in the liver. Proc Natl Acad Sci USA 2008; 105:9733-8.
33. Luedde T, Beraza N, Kotsikoris V, et al. Deletion of NEMO/IKKgamma in liver parenchymal cells causes steatohepatitis and hepatocellular carcinoma. Cancer Cell 2007; 11:119-32.
34. Greten F R, Eckmann L, Greten T F, et al. IKKbeta links inflammation and tumorigenesis in a mouse model of colitis-associated cancer. Cell 2004; 118:285-96.
35. Vema L, Whysner J, Williams GM. N-nitrosodiethylamine mechanistic data and risk assessment: bioactivation, DNA-adduct formation, mutagenicity, and tumor initiation. Pharmacol Ther 1996; 71:57-81.
36. Lai D Y, Arcos J C. Minireview: dialkylnitrosamine bioactivation and carcinogenesis. Life Sci 1980; 27:2149-65.
37. Magee P N, Barnes J M. Carcinogenic nitroso compounds. Adv Cancer Res 1967; 10:163-246.
38. Fausto N. Mouse liver tumorigenesis: models, mechanisms, and relevance to human disease. Semin Liver Dis 1999; 19:243-52.
39. Maeda S, Kamata H, Luo J L, Leffert H, Karin M. IKKbeta couples hepatocyte death to cytokine-driven compensatory proliferation that promotes chemical hepatocarcinogenesis. Cell 2005; 121:977-90.
40. Luo J L, Kamata H, Karin M. IKK/NF-kappaB signaling: balancing life and death—a new approach to cancer therapy. J Clin Invest 2005; 115:2625-32.
41. Kamata H, Honda S, Maeda S, Chang L, Hirata H, Karin M. Reactive oxygen species promote TNFalpha-induced death and sustained JNK activation by inhibiting MAP kinase phosphatases. Cell 2005; 120:649-61.
42. Pham C G, Bubici C, Zazzeroni F, et al. Ferritin heavy chain upregulation by NF-kappaB inhibits TNFalpha-induced apoptosis by suppressing reactive oxygen species. Cell 2004; 119:529-42.
43. Sakurai T, Maeda S, Chang L, Karin M. Loss of hepatic NF-kappa B activity enhances chemical hepatocarcinogenesis through sustained c-Jun N-terminal kinase 1 activation. Proc Natl Acad Sci USA 2006; 103:10544-51.
44. Pikarsky E, Porat R M, Stein I, et al. NF-kappaB functions as a tumour promoter in inflammation-associated cancer. Nature 2004; 431:461-6.
45. Mauad T H, van Nieuwkerk C M, Dingemans K P, et al. Mice with homozygous disruption of the mdr2 P-glycoprotein gene. A novel animal model for studies of nonsuppurative inflammatory cholangitis and hepatocarcinogenesis. Am J Pathol 1994; 145:1237-45.
46. Haybaeck J, Zeller N, Wolf M J, et al. A lymphotoxin-driven pathway to hepatocellular carcinoma. Cancer Cell 2009; 16:295-308.
47. Maeda S, Hikiba Y, Sakamoto K, et al. Ikappa B kinasebeta/nuclear factor-kappaB activation controls the development of liver metastasis by way of interleukin-6 expression. Hepatology 2009; 50:1851-60.
48. Sakurai T, He G, Matsuzawa A, et al. Hepatocyte necrosis induced by oxidative stress and IL-1 alpha release mediate carcinogen-induced compensatory proliferation and liver tumorigenesis. Cancer Cell 2008; 14:156-65.
49. Naugler W E, Sakurai T, Kim S, et al. Gender disparity in liver cancer due to sex differences in MyD88-dependent IL-6 production. Science 2007; 317:121-4.
50. Jilka R L, Hangoc G, Girasole G, et al. Increased osteoclast development after estrogen loss: mediation by interleukin-6. Science 1992; 257:88-91.
51. Ershler W B, Keller E T. Age-associated increased interleukin-6 gene expression, late-life diseases, and frailty. Annu Rev Med 2000; 51:245-70.
52. Tilg H, Wilmer A, Vogel W, et al. Serum levels of cytokines in chronic liver diseases. Gastroenterology 1992; 103:264-74.
53. Trikha M, Corringham R, Klein B, Rossi J F. Targeted anti-interleukin-6 monoclonal antibody therapy for cancer: a review of the rationale and clinical evidence. Clin Cancer Res 2003; 9:4653-65.
54. Nakagawa H, Maeda S, Yoshida H, et al. Serum IL-6 levels and the risk for hepatocarcinogenesis in chronic hepatitis C patients: an analysis based on gender differences. Int J Cancer 2009; 125:2264-9.
55. Wong V W, Yu J, Cheng A S, et al. High serum interleukin-6 level predicts future hepatocellular carcinoma development in patients with chronic hepatitis B. Int Cancer 2009; 124:2766-70.
56. Wegenka U M, Buschmann J, Lutticken C, Heinrich P C, Horn F. Acute-phase response factor, a nuclear factor binding to acute-phase response elements, is rapidly activated by interleukin-6 at the posttranslational level. Mol Cell Biol 1993; 13:276-88.
57. Zhong Z, Wen Z, Darnell J E, Jr. Stat3: a STAT family member activated by tyrosine phosphorylation in response to epidermal growth factor and interleukin-6. Science 1994; 264:95-8.
58. Takeda K, Akira S. STAT family of transcription factors in cytokine-mediated biological responses. Cytokine Growth Factor Rev 2000; 11:199-207.
59. Hirano T, Ishihara K, Hibi M. Roles of STAT3 in mediating the cell growth, differentiation and survival signals relayed through the IL-6 family of cytokine receptors. Oncogene 2000; 19:2548-56.
60. Yoshimura A, Naka T, Kubo M. SOCS proteins, cytokine signalling and immune regulation. Nat Rev Immunol 2007; 7:454-65.
61. Wen Z, Zhong Z, Darnell J E, Jr. Maximal activation of transcription by Stat1 and Stat3 requires both tyrosine and serine phosphorylation. Cell 1995; 82:241-50.
62. Yuan Z L, Guan Y J, Chatterjee D, Chin Y E. Stat3 dimerization regulated by reversible acetylation of a single lysine residue. Science 2005; 307:269-73.
63. Kubo M, Hanada T, Yoshimura A. Suppressors of cytokine signaling and immunity. Nat Immunol 2003; 4:1169-76.
64. Al Zaid Siddiquee K, Turkson J. STAT3 as a target for inducing apoptosis in solid and hematological tumors. Cell Res 2008; 18:254-67.
65. He G, Yu G Y, Temkin V, et al. Hepatocyte IKKbeta/NF-kappaB inhibits tumor promotion and progression by preventing oxidative stress-driven STAT3 activation. Cancer Cell 2010; 17:286-97.
66. Calvisi D F, Ladu S, Gorden A, et al. Ubiquitous activation of Ras and Jak/Stat pathways in human HCC. Gastroenterology 2006; 130:1117-28.
67. Rebouissou S, Amessou M, Couchy G, et al. Frequent in-frame somatic deletions activate gp130 in inflammatory hepatocellular tumours. Nature 2009; 457:200-4.
68. El-Serag H B, Rudolph K L. Hepatocellular carcinoma: epidemiology and molecular carcinogenesis. Gastroenterology 2007; 132:2557-76.
69. Parekh S, Anania F A. Abnormal lipid and glucose metabolism in obesity: implications for nonalcoholic fatty liver disease. Gastroenterology 2007; 132:2191-207.
70. Wang T, Weinman S A. Causes and consequences of mitochondrial reactive oxygen species generation in hepatitis C. J Gastroenterol Hepatol 2006; 21 Suppl 3:S34-7.
71. Takeda K, Noguchi K, Shi W, et al. Targeted disruption of the mouse Stat3 gene leads to early embryonic lethality. Proc Natl Acad Sci USA 1997; 94:3801-4.
72. Raz R, Lee C K, Cannizzaro L A, d'Eustachio P, Levy D E. Essential role of STAT3 for embryonic stem cell pluripotency. Proc Natl Acad Sci USA 1999; 96:2846-51.
73. Matsuda T, Nakamura T, Nakao K, et al. STAT3 activation is sufficient to maintain an undifferentiated state of mouse embryonic stem cells. Embo J 1999; 18:4261-9.
74. Levy D E, Lee C K. What does Stat3 do? J Clin Invest 2002; 109:1143-8.
75. Chan K S, Sano S, Kiguchi K, et al. Disruption of Stat3 reveals a critical role in both the initiation and the promotion stages of epithelial carcinogenesis. J Clin Invest 2004; 114:720-8.
76. Bollrath J, Phesse T J, von Burstin V A, et al. gp130-mediated Stat3 activation in enterocytes regulates cell survival and cell-cycle progression during colitis-associated tumorigenesis. Cancer Cell 2009; 15:91-102.
77. Grivennikov S, Karin E, Terzic J, et al. IL-6 and Stat3 are required for survival of intestinal epithelial cells and development of colitis-associated cancer. Cancer Cell 2009; 15:103-13.
78. Inghirami G, Chiarle R, Simmons W J, Piva R, Schlessinger K, Levy D E. New and old functions of STAT3: a pivotal target for individualized treatment of cancer. Cell Cycle 2005; 4:1131-3.
79. Chiarle R, Simmons W J, Cai H, et al. Stat3 is required for ALK-mediated lymphomagenesis and provides a possible therapeutic target. Nat Med 2005; 11:623-9.
80. Yue P, Turkson J. Targeting STAT3 in cancer: how successful are we? Expert Opin Investig Drugs 2009; 18:45-56.
81. Fletcher S, Drewry J A, Shahani V M, Page B D, Gunning P T. Molecular disruption of oncogenic signal transducer and activator of transcription 3 (STAT3) protein. Biochem Cell Biol 2009; 87:825-33.
82. Siddiquee K, Zhang S, Guida W C, et al. Selective chemical probe inhibitor of Stat3, identified through structure-based virtual screening, induces antitumor activity. Proc Natl Acad Sci USA 2007; 104:7391-6.
83. Lin L, Amin R, Gallicano G I, et al. The STAT3 inhibitor NSC 74859 is effective in hepatocellular cancers with disrupted TGF-beta signaling. Oncogene 2009; 28:961-72.
84. Meydan N, Grunberger T, Dadi H, et al. Inhibition of acute lymphoblastic leukaemia by a Jak-2 inhibitor. Nature 1996; 379:645-8.
85. Yang J, Chatterjee-Kishore M, Staugaitis S M, et al. Novel roles of unphosphorylated STAT3 in oncogenesis and transcriptional regulation. Cancer Res 2005; 65:939-47.
86. Li W C, Ye S L, Sun R X, et al. Inhibition of growth and metastasis of human hepatocellular carcinoma by antisense oligonucleotide targeting signal transducer and activator of transcription 3. Clin Cancer Res 2006; 12:7140-8.
87. Hedvat M, Huszar D, Herrmann A, et al. The JAK2 inhibitor AZD1480 potently blocks Stat3 signaling and oncogenesis in solid tumors. Cancer Cell 2009; 16:487-97.
88. Grivennikov S I, Karin M. Dangerous liaisons: STAT3 and NF-kappaB collaboration and crosstalk in cancer. Cytokine Growth Factor Rev; 21:11-9.
89. Atkinson G P, Nozell S E, Benveniste E T. NF-kappaB and STAT3 signaling in glioma: targets for future therapies. Expert Rev Neurother; 10:575-86.
90. Bollrath J, Greten F R. IKK/NF-kappaB and STAT3 pathways: central signalling hubs in inflammation-mediated tumour promotion and metastasis. EMBO Rep 2009; 10:1314-9.
91. Valentino L, Pierre J. JAK/STAT signal transduction: regulators and implication in hematological malignancies. Biochem Pharmacol 2006; 71:713-21.
92. Hsu L-C, Enzler T, Seita J, et al. Increased interleukin 1β-driven neutrophil counts preserve anti-bacterial defense in absence of IKKβ. Nat Immunol 2010; In press.
93. Lee H, Herrmann A, Deng J H, et al. Persistently activated Stat3 maintains constitutive NF-kappaB activity in tumors. Cancer Cell 2009; 15:283-93.
94. Chen L, Fischle W, Verdin E, Greene W C. Duration of nuclear NF-kappaB action regulated by reversible acetylation. Science 2001; 293:1653-7.
95. Bruix J, Sherman M. Management of hepatocellular carcinoma. Hepatology 2005; 42:1208-36.
96. Park E J, Lee J H, Yu G Y, et al. Dietary and genetic obesity promote liver inflammation and tumorigenesis by enhancing IL-6 and TNF expression. Cell 2010; 140:197-208.

### REFERENCES CITED IN THE ABOVE “EXPERIMENTAL” EXAMPLES 1-10

1. El-Serag, H. B. & Rudolph, K. L. Hepatocellular carcinoma: epidemiology and molecular carcinogenesis. Gastroenterology 132, 2557-2576 (2007).
2. Parkin, D. M. Global cancer statistics in the year 2000. Lancet Oncol 2, 533-543 (2001).
3. Cancer Facts & Figures 2005. ACS Online: 1-60 (American Cancer Society, Inc, Atlanta, Ga., 2005).
4. Farazi, P. A. & DePinho, R. A. Hepatocellular carcinoma pathogenesis: from genes to environment. Nat Rev Cancer 6, 674-687 (2006).
5. Parkin, D. M., Whelan, S. L., Ferlay, J., Teppo, L. & Thomas, D. B. (eds.). Cancer incidence in five continents., 1-781 (International Agency for Research on Cancer, Lyon, France, 2002).
6. Calle, E. E., Rodriguez, C., Walker-Thurmond, K. & Thun, M. J. Overweight, obesity, and mortality from cancer in a prospectively studied cohort of U.S. adults. N Engl J Med 348, 1625-1638 (2003).
7. Zender, L., et al. Cancer gene discovery in hepatocellular carcinoma. J Hepatol 52, 921-929 (2010).
8. Rebouissou, S., et al., Frequent in-frame somatic deletions activate gp130 in inflammatory hepatocellular tumours. Nature 457, 200-204 (2009).
9. Sell, S. & Leffert, H. L. Liver cancer stem cells. J Clin Oncol 26, 2800-2805 (2008).
10. Yang, Z. F., et al. Significance of CD90+ cancer stem cells in human liver cancer. Cancer Cell 13, 153-166 (2008).
11. Clevers, H. Wnt/beta-catenin signaling in development and disease. Cell 127, 469-480 (2006).
12. Li, Y. Q. Master stem cell transcription factors and signaling regulation. Cell Reprogram 12, 3-13 (2010).
13. Cieply, B., Zeng, G., Proverbs-Singh, T., Geller, D. A. & Monga, S. P. Unique phenotype of hepatocellular cancers with exon-3 mutations in beta-catenin gene. Hepatology 49, 821-831 (2009).
14. Thompson, M. D. & Monga, S. P. WNT/beta-catenin signaling in liver health and disease. Hepatology 45, 1298-1305 (2007).
15. Yamashita, T., et al. EpCAM and alpha-fetoprotein expression defines novel prognostic subtypes of hepatocellular carcinoma. Cancer Res 68, 1451-1461 (2008).
16. Yamashita, T., Budhu, A., Forgues, M. & Wang, X. W. Activation of hepatic stem cell marker EpCAM by Wnt-beta-catenin signaling in hepatocellular carcinoma. Cancer Res 67, 10831-10839 (2007).
17. Yamashita, T., et al. EpCAM-positive hepatocellular carcinoma cells are tumor-initiating cells with stem/progenitor cell features. Gastroenterology 136, 1012-1024 (2009).
18. Ma, S., et al., Identification and characterization of tumorigenic liver cancer stem/progenitor cells. Gastroenterology 132, 2542-2556 (2007).
19. Suetsugu, A., et al., Characterization of CD133+ hepatocellular carcinoma cells as cancer stem/progenitor cells. Biochem Biophys Res Commun 351, 820-824 (2006).
20. Zulehner, G., et al., Nuclear beta-catenin induces an early liver progenitor phenotype in hepatocellular carcinoma and promotes tumor recurrence. Am J Pathol 176, 472-481 (2010).
21. Zaret, K. S. & Grompe, M. Generation and regeneration of cells of the liver and pancreas. Science 322, 1490-1494 (2008).
22. Chen, X., et al. Integration of external signaling pathways with the core transcriptional network in embryonic stem cells. Cell 133, 1106-1117 (2008).
23. Yao, Z. & Mishra, L. Cancer stem cells and hepatocellular carcinoma. Cancer Biol Ther 8, 1691-1698 (2009).
24. He, G., et al. Hepatocyte IKKbeta/NF-kappaB inhibits tumor promotion and progression by preventing oxidative stress-driven STAT3 activation. Cancer Cell 17, 286-297 (2010).
25. Calvisi, D. F., et al., Ubiquitous activation of Ras and Jak/Stat pathways in human HCC. Gastroenterology 130, 1117-1128 (2006).
26. Lin, L., et al. The STAT3 inhibitor NSC 74859 is effective in hepatocellular cancers with disrupted TGF-beta signaling. Oncogene 28, 961-972 (2009).
27. Hatziapostolou, M., et al. An HNF4a-miRNA Inflammatory Feedback Circuit regulates
Hepatocellular Oncogenesis. Cell In Press (2011).
28. Bromberg, J. & Wang, T. C. Inflammation and cancer: IL-6 and STAT3 complete the link. Cancer Cell 15, 79-80 (2009).
29. Naugler, W. E. & Karin, M. The wolf in sheep's clothing: the role of interleukin-6 in immunity, inflammation and cancer. Trends Mol Med 14, 109-119 (2008).
30. Hoshida, Y., et al. Gene expression in fixed tissues and outcome in hepatocellular carcinoma. N Engl J Med 359, 1995-2004 (2008).
31. Nakagawa, H., et al., Serum IL-6 levels and the risk for hepatocarcinogenesis in chronic hepatitis C patients: an analysis based on gender differences. Int J Cancer 125, 2264-2269 (2009).
32. Wong, V. W., et al. High serum interleukin-6 level predicts future hepatocellular carcinoma development in patients with chronic hepatitis B. Int J Cancer (2009).
33. Verna, L., Whysner, J. & Williams, G. M. N-nitrosodiethylamine mechanistic data and risk assessment: bioactivation, DNA-adduct formation, mutagenicity, and tumor initiation. Pharmacol Ther 71, 57-81 (1996).
34. Maeda, S., Kamata, H., Luo, J. L., Leffert, H. & Karin, M. IKKbeta couples hepatocyte death to cytokine-driven compensatory proliferation that promotes chemical hepatocarcinogenesis. Cell 121, 977-990 (2005).
35. Karin, M. Nuclear factor-kappaB in cancer development and progression. Nature 441, 431-436 (2006).
36. Sakurai, T., Maeda, S., Chang, L. & Karin, M. Loss of hepatic NF-kappa B activity enhances chemical hepatocarcinogenesis through sustained c-Jun N-terminal kinase 1 activation. Proc Natl Acad Sci USA 103, 10544-10551 (2006).
37. Wang, T. & Weinman, S. A. Causes and consequences of mitochondrial reactive oxygen species generation in hepatitis C. J Gastroenterol Hepatol 21 Suppl 3, S34-37 (2006).
38. Naugler, W. E., et al. Gender disparity in liver cancer due to sex differences in MyD88-dependent IL-6 production. Science 317, 121-124 (2007).
39. Park, E. J., et al., Dietary and genetic obesity promote liver inflammation and tumorigenesis by enhancing IL-6 and TNF expression. Cell 140, 197-208 (2010).
40. Toffanin, S., Friedman, S. L. & Llovet, J. M. Obesity, inflammatory signaling, and hepatocellular carcinoma-an enlarging link. Cancer Cell 17, 115-117 (2010).
41. Weglarz, T. C., Degen, J. L. & Sandgren, E. P. Hepatocyte transplantation into diseased mouse liver. Kinetics of parenchymal repopulation and identification of the proliferative capacity of tetraploid and octaploid hepatocytes. Am J Pathol 157, 1963-1974 (2000).
42. Laconi, E., et al. Long-term, near-total liver replacement by transplantation of isolated hepatocytes in rats treated with retrorsine. Am J Pathol 153, 319-329 (1998).
43. Inokuchi, S., et al., Disruption of TAK1 in hepatocytes causes hepatic injury, inflammation, fibrosis, and carcinogenesis. Proc Natl Acad Sci USA 107, 844-849 (2010).
44. Bannasch, P. Sequential cellular changes during chemical carcinogenesis. J Cancer Res Clin Oncol 108, 11-22 (1984).
45. Rabes, H. M. Development and growth of early preneoplastic lesions induced in the liver by chemical carcinogens. J Cancer Res Clin Oncol 106, 85-92 (1983).
46. Wanless, I. R., et al., Hepatic and portal vein thrombosis in cirrhosis: possible role in development of parenchymal extinction and portal hypertension. Hepatology 21, 1238-1247 (1995).
47. Pitot, N. C. Altered hepatic foci: their role in murine hepatocarcinogenesis. Annu Rev Pharmacol Toxicol, 30, 465-500 (1990).
48. Hytiroglou, P., Park, Y. N., Krinsky, G. & Theise, N. D. Hepatic precancerous lesions and small hepatocellular carcinoma. Gastroenterol Clin North Am 36, 867-887, vii (2007).
49. Seki, S., et al. Outcomes of dysplastic nodules in human cirrhotic liver: a clinicopathological study. Clin Cancer Res 6, 3469-3473 (2000).
50. Takayama, T., et al. Malignant transformation of adenomatous hyperplasia to hepatocellular carcinoma. Lancet 336, 1150-1153 (1990).
51. Yang, W., et al. Wnt/beta-catenin signaling contributes to activation of normal and tumorigenic liver progenitor cells. Cancer Res 68, 4287-4295 (2008).
52. Sakurai, T., et al., Hepatocyte necrosis induced by oxidative stress and IL-1 alpha release mediate carcinogen-induced compensatory proliferation and liver tumorigenesis. Cancer Cell 14, 156-165 (2008).
53. Zhu, Z., et al. Cancer stem/progenitor cells are highly enriched in CD133+CD44+ population in hepatocellular carcinoma. Int J Cancer 126, 2067-2078 (2010).
54. Kon, J., Ooe, H., Oshima, H., Kikkawa, Y. & Mitaka, T. Expression of CD44 in rat hepatic progenitor cells. J Hepatol 45, 90-98 (2006).
55. Turner, W. S., at al. Nuclear magnetic resonance metabolomic footprinting of human hepatic stem cells and hepatoblasts cultured in hyaluronan-matrix hydrogels. Stem Cells 26, 1547-1555 (2008).
56. Zhang, L., Theise, N., Chua, M. & Reid, L. M. The stem cell niche of human livers: symmetry between development and regeneration. Hepatology 48, 1598-1607 (2008).
57. Erker, L. & Grompe, M. Signaling networks in hepatic oval cell activation. Stem Cell Res 1, 90-102 (2007).
58. Huang, D. W., Sherman, B. T. & Lempicki, R. A. Systematic and integrative analysis of large gene lists using DAVID bioinformatics resources. Nat Protoc 4, 44-57 (2009).
59. Huang, D. W., Sherman, B. T. & Lempicki, R. A. Bioinformatics enrichment tools: paths toward the comprehensive functional analysis of large gene lists. Nucleic Acids Res 37, 1-13 (2009).
60. Shin, S., et al. FoxI1-Cre-marked adult hepatic progenitors have clonogenic and bilineage differentiation potential. Genes Dev 25, 1185-1192 (2011).
61. Luedde, T. & Schwabe, R. F. NF-kappaB in the liver-linking injury, fibrosis and hepatocellular carcinoma. Nat Rev Gastroenterol Hepatol 8, 108-118 (2011).
62. Dorrell, C., et al. Prospective isolation of a bipotential clonogenic liver progenitor cell in adult mice. Genes Dev 25, 1193-1203 (2011).
63. Espina, V., et al. Laser-capture microdissection. Nat Protoc 1, 586-603 (2006).
64. Ponta, H., Sherman, L. & Herrlich, P. A. CD44: from adhesion molecules to signalling regulators. Nat Rev Mol Cell Blot 4, 33-45 (2003).
65. Tan, W., et al. Tumour-infiltrating regulatory T cells stimulate mammary cancer metastasis through RANKL-RANK signalling. Nature 470, 548-553 (2011).
66. Ammirante, M., Luo, J. L., Grivennikov, S., Nedospasov, S. & Karin, M. B-cell-derived lymphotoxin promotes castration-resistant prostate cancer. Nature 464, 302-305 (2010).
67. Jha, R. K., Ma, Q., Chen, S., Sha, H. & Ding, S. Relationship of fibronectin and CD44v6 expression with invasive growth and metastasis of liver cancer. Cancer Invest 27, 324-328 (2009).
68. Chiu, C. C., et al. Global gene expression profiling reveals a key role of CD44 in hepatic oval-cell reaction after 2-AAF/CCl4 injury in rodents. Histochem Cell Biol 132, 479-489 (2009).
69. Monga, S. P., Pediaditakis, P., Mule, K., Stolz, D. B. & Michalopoulos, G. K. Changes in WNT/beta-catenin pathway during regulated growth in rat liver regeneration. Hepatology 33, 1098-1109 (2001).
70. Ishizaki, Y., et al. Immunohistochemical analysis and mutational analyses of beta-catenin, Axin family and APC genes in hepatocellular carcinomas. Int J Oncol 24, 1077-1083 (2004).
71. Zhang, X. F., et al., Conditional beta-catenin loss in mice promotes chemical hepatocarcinogenesis: role of oxidative stress and platelet-derived growth factor receptor alpha/phosphoinositide 3-kinase signaling. Hepatology 52, 954-965 (2010).
72. Seymour, P. A., et al. SOX9 is required for maintenance of the pancreatic progenitor cell pool. Proc Natl Acad Sci USA 104, 1865-1870 (2007).
73. Vidal, V. P., et al. Sox9 is essential for outer root sheath differentiation and the formation of the hair stem cell compartment. Curr Biol 15, 1340-1351 (2005).
74. Furuyama, K., et al. Continuous cell supply from a Sox9-expressing progenitor zone in adult liver, exocrine pancreas and intestine. Nat Genet. 43, 34-41 (2010).
75. Kist, R., Schrewe, H., Balling, R. & Scherer, G. Conditional inactivation of Sox9: a mouse model for campomelic dysplasia. Genesis 32, 121-123 (2002).
76. Kopp, J. L., et al. Sox9+ ductal cells are multipotent progenitors throughout development but do not produce new endocrine cells in the normal or injured adult pancreas. Development 138, 653-665 (2011).
77. Sicklick, J. K., et al. Hedgehog signaling maintains resident hepatic progenitors throughout life. Am J Physiol Gastrointest Liver Physiol 290, G859-870 (2006).
78. Abrahamsson, A. E., et al., Glycogen synthase kinase 3beta missplicing contributes to leukemia stem cell generation. Proc Natl Acad Sci USA 106, 3925-3929 (2009).
79. Alley, S. C., Okeley, N. M. & Senter, P. D. Antibody-drug conjugates: targeted drug delivery for cancer. Curr Opin Chem Biol 14, 529-537 (2010).
80. Burns, H A., 3rd, et al., Phase II Study of the Antibody Drug Conjugate Trastuzumab-DM1 for the Treatment of Human Epidermal Growth Factor Receptor 2 (HER2)-Positive Breast Cancer After Prior HER2-Directed Therapy. J Clin Oncol (2010).
81. Junttila, T. T., Li, G., Parsons, K., Phillips, G. L. & Sliwkowski, M. X. Trastuzumab-DM1 (T-DM1) retains all the mechanisms of action of trastuzumab and efficiently inhibits growth of lapatinib insensitive breast cancer. Breast Cancer Res Treat (2010).
82. Cassady, J. M., Chan, K. K., Floss, H. G. & Leistner, E. Recent developments in the maytansinoid antitumor agents. Chem Pharm Bull (Tokyo) 52, 1-26 (2004).
83. Lopus, M., et al., Maytansine and cellular metabolites of antibody-maytansinoid conjugates strongly suppress microtubule dynamics by binding to microtubules. Mol Cancer Ther 9, 2689-2699 (2010).
84. Hamel, W., Magnelli, L., Chiarugi, V. P. & Israel, M. A. Herpes simplex virus thymidine kinase/ganciclovir-mediated apoptotic death of bystander cells. Cancer Res 56, 2697-2702 (1996).
85. Kanai, F., et al. Gene therapy for alpha-fetoprotein-producing human hepatoma cells by adenovirus-mediated transfer of the herpes simplex virus thymidine kinase gene. Hepatology 23, 1359-1368 (1996).
86. Hirano, T., et al. HVJ-liposome-mediated transfection of HSVtk gene driven by AFP promoter inhibits hepatic tumor growth of hepatocellular carcinoma in SCID mice. Gene Ther 8, 80-83 (2001).
87. Li, L. Y., et al. Targeted hepatocellular carcinoma proapoptotic BikDD gene therapy. Oncogene (2010).
88. Mohr, L., et al. Cationic liposome-mediated gene delivery to the liver and to hepatocellular carcinomas in mice. Hum Gene Ther 12, 799-809 (2001).
89. Siwak, D. R., Tari, A. M. & Lopez-Berestein, G. The potential of drug-carrying immunoliposomes as anticancer agents. Commentary re: J. W. Park et al., Anti-HER2 immunoliposomes: enhanced efficacy due to targeted delivery. Clin. Cancer Res., 8: 1172-1181, 2002. Clin Cancer Res 8, 955-956 (2002).

### REFERENCES CITED IN THE ABOVE “EXPERIMENTAL” EXAMPLE 11

Budanov, A. V., and Karin, M. (2008). p53 target genes sestrin1 and sestrin2 connect genotoxic stress and mTOR signaling. Cell 134, 451-460.
Formaro, M., Burch, P. M., Yang, W., Zhang, L., Hamilton, C. E., Kim, J. H., Neel, B. G., and Bennett, A. M. (2006). SHP-2 activates signaling of the nuclear factor of activated T cells to promote skeletal muscle growth. J Cell Biol 175, 87-97.
Lee, K., and Esselman, W. J. (2002). Inhibition of PTPs by H(2)O(2) regulates the activation of distinct MAPK pathways. Free Radic Biol Med 33, 1121-1132.
Leffert, H. L., Koch, K. S., Moran, T., and Williams, M. (1979). Liver cells. Methods Enzymol 58, 536-544.
Park, J. M., Greten, F. R., Li, Z. W., and Karin, M. (2002). Macrophage apoptosis by anthrax lethal factor through p38 MAP kinase inhibition. Science 297, 2048-2051.
Sieghart, W., Losert, D., Strommer, S., Cejka, D., Schmid, K., Rasoul-Rockenschaub, S., Bodingbauer, M., Crevenna, R., Monia, B. P., Peck-Radosavljevic, M., and Wacheck, V. (2006). Mcl-1 overexpression in hepatocellular carcinoma: a potential target for antisense therapy. J Hepatol 44, 151-157.

### REFERENCES CITED IN THE ABOVE “EXPERIMENTAL” EXAMPLES 12-16 AND “DESCRIPTION OF THE INVENTION” N

Al-Hajj, M., and Clarke, M. F. (2004). Self-renewal and solid tumor stem cells. Oncogene 23, 7274-7282.
Albini, A., and Sporn, M. B. (2007). The tumour microenvironment as a target for chemoprevention. Nat Rev Cancer 7, 139-147.
Auernhammer, C. J., Bousquet, C., and Melmed, S. (1999). Autoregulation of pituitary corticotroph SOCS-3 expression: characterization of the murine SOCS-3 promoter. Proc Natl Acad Sci USA 96, 6964-6969.
Berasain, C., Castillo, J., Perugorria, M. J., Latasa, M. U., Prieto, J., and Avila, M. A. (2009). Inflammation and liver cancer. Ann N Y Acad Sci 1155, 206-221.
Boehm, J. S., Zhao, J. J., Yao, J., Kim, S. Y., Firestein, R., Dunn, I. F., Sjostrom, S. K., Garraway, L. A., Weremowicz, S., Richardson, A. L., et al., (2007). Integrative genomic approaches identify IKBKE as a breast cancer oncogene. Cell 129, 1065-1079.
Bollrath, J., Phesse, T. J., von Burstin, V. A., Putoczki, T., Bennecke, M., Bateman, T., Nebelsiek, T., Lundgren-May, T., Canli, O., Schwitalla, S., et al. (2009). gp130-mediated Stat3 activation in enterocytes regulates cell survival and cell-cycle progression during colitis-associated tumorigenesis. Cancer Cell 15, 91-102.
Bosch, F. X., Ribes, J., Diaz, M., and Cleries, R. (2004). Primary liver cancer: worldwide incidence and trends. Gastroenterology 127, S5-516.
Boucher, K. M., and Yakovlev, A. Y. (1997). Estimating the probability of initiated cell death before tumor induction. Proc Natl Acad Sci USA 94, 12776-12779.
Brender, C., Nielsen, M., Kaltoft, K., Mikkelsen, G., Zhang, Q., Wasik, M., Billestrup, N., and Odum, N. (2001). STAT3-mediated constitutive expression of SOCS-3 in cutaneous T-cell lymphoma. Blood 97, 1056-1062.
Bromberg, J., and Wang, T. C. (2009). Inflammation and cancer: IL-6 and STAT3 complete the link. Cancer Cell 15, 79-80.
Calvisi, D. F., Ladu, S., Gorden, A., Farina, M., Conner, E. A., Lee, J. S., Factor, V. M., and Thorgeirsson, S. S. (2006). Ubiquitous activation of Ras and Jak/Stat pathways in human HCC. Gastroenterology 130, 1117-1128.
Calvisi, D. F., and Thorgeirsson, S. S. (2005). Molecular mechanisms of hepatocarcinogenesis in transgenic mouse models of liver cancer. Toxicol Pathol 33, 181-184.
Chan, K. S., Sano, S., Kiguchi, K., Anders, J., Komazawa, N., Takeda, J., and DiGiovanni, J. (2004). Disruption of Stat3 reveals a critical role in both the initiation and the promotion stages of epithelial carcinogenesis. J Clin Invest 114, 720-728.
Chen, C. H., Huang, G. T., Yang, P. M., Chen, P. J., Lai, M. Y., Chen, D. S., Wang, J. D., and Sheu, J. C. (2006). Hepatitis B- and C-related hepatocellular carcinomas yield different clinical features and prognosis. Eur J Cancer 42, 2524-2529.
Cioffi, M., Esposito, K., Vietri, M. T., Gazzerro, P., D'Auria, A., Ardovino, I., Puca, G. A., and Molinari, A. M. (2002). Cytokine pattern in postmenopause. Maturitas 41, 187-192.
Coussens, L. M., and Werb, Z. (2002). Inflammation and cancer. Nature 420, 860-867.
Dajee, M., Lazarov, M., Zhang, J. Y., Cai, T., Green, C. L., Russell, A. J., Marinkovich, M. P., Tao, S., Lin, Q., Kubo, Y., and Khavari, P. A. (2003). NF-kappaB blockade and oncogenic Ras trigger invasive human epidermal neoplasia. Nature 421, 639-643.
El-Serag, H. B., and Rudolph, K. L. (2007). Hepatocellular carcinoma: epidemiology and molecular carcinogenesis. Gastroenterology 132, 2557-2576.
Eriksen, K. W., Kaltoft, K., Mikkelsen, G., Nielsen, M., Zhang, Q., Geisler, C., Nissen, M. H., Ropke, C., Wasik, M. A., and Odum, N. (2001). Constitutive STAT3-activation in Sezary syndrome: tyrphostin AG490 inhibits STAT3-activation, interleukin-2 receptor expression and growth of leukemic Sezary cells. Leukemia 15, 787-793.
Fausto, N. (1999). Mouse liver tumorigenesis: models, mechanisms, and relevance to human disease. Semin Liver Dis 19, 243-252.
Greten, F. R., Eckmann, L., Greten, T. F., Park, J. M., Li, Z. W., Egan, L. J., Kagnoff, M. F., and Karin, M. (2004). IKKbeta links inflammation and tumorigenesis in a mouse model of colitis-associated cancer. Cell 118, 285-296.
Grivennikov, S., Karin, E., Terzic, J., Mucida, D., Yu, G. Y., Vallabhapurapu, S., Scheller, J., Rose-John, S., Cheroutre, H., Eckmann, L., and Karin, M. (2009). IL-6 and Stat3 are required for survival of intestinal epithelial cells and development of colitis-associated cancer. Cancer Cell 15, 103-113.
Haybaeck, J., Zeller, N., Wolf, M. J., Weber, A., Wagner, U., Kurrer, M. O., Bremer, J., Iezzi, G., Graf, R., Clavien, P. A., et al. (2009). A lymphotoxin-driven pathway to hepatocellular carcinoma. Cancer Cell 16, 295-308.
Hedvat, M., Huszar, D., Herrmann, A., Gozgit, J. M., Schroeder, A., Sheehy, A., Buettner, R., Proia, D., Kowolik, C. M., Xin, H., et al. (2009). The JAK2 inhibitor AZD1480 potently blocks Stat3 signaling and oncogenesis in solid tumors. Cancer Cell 16, 487-497.
Hennings, H., Glick, A. B., Greenhalgh, D. A., Morgan, D. L., Strickland, J. E., Tennenbaum, T., and Yuspa, S. H. (1993). Critical aspects of initiation, promotion, and progression in multistage epidermal carcinogenesis. Proc Soc Exp Biol Med 202, 1-8.
Hytiroglou, P., Park, Y. N., Krinsky, G., and Theise, N. D. (2007). Hepatic precancerous lesions and small hepatocellular carcinoma. Gastroenterol Clin North Am 36, 867-887, vii.
Kamata, H., Honda, S., Maeda, S., Chang, L., Hirata, H., and Karin, M. (2005). Reactive oxygen species promote TNFalpha-induced death and sustained JNK activation by inhibiting MAP kinase phosphatases. Cell 120, 649-661.
Kamimura, D., Ishihara, K., and Hirano, T. (2003). IL-6 signal transduction and its physiological roles: the signal orchestration model. Rev Physiol Biochem Pharmacol 149, 1-38.
Karin, M. (2006). Nuclear factor-kappaB in cancer development and progression. Nature 441, 431-436.
Kaufmann, W. K., Ririe, D. G., and Kaufman, D. G. (1988). Phenobarbital-dependent proliferation of putative initiated rat hepatocytes. Carcinogenesis 9, 779-782.
Kuhn, R., Schwenk, F., Aguet, M., and Rajewsky, K. (1995). Inducible gene targeting in mice. Science 269, 1427-1429.
Lee, C. K., Raz, R., Gimeno, R., Gertner, R., Wistinghausen, B., Takeshita, K., DePinho, R. A., and Levy, D. E. (2002). STAT3 is a negative regulator of granulopoiesis but is not required for G-CSF-dependent differentiation. Immunity 17, 63-72.
Lin, L., Amin, R., Gallicano, G. I., Glasgow, E., Jogunoori, W., Jessup, J. M., Zasloff, M., Marshall, J. L., Shetty, K., Johnson, L., et al. (2009). The STAT3 inhibitor NSC 74859 is effective in hepatocellular cancers with disrupted TGF-beta signaling. Oncogene 28, 961-972.
Luedde, T., Beraza, N., Kotsikoris, V., van Loo, G., Nenci, A., De Vos, R., Roskams, T., Trautwein, C., and Pasparakis, M. (2007). Deletion of NEMO/IKKgamma in liver parenchymal cells causes steatohepatitis and hepatocellular carcinoma. Cancer Cell 11, 119-132.
Maeda, S., Kamata, H., Luo, J. L., Leffert, H., and Karin, M. (2005). IKKbeta couples hepatocyte death to cytokine-driven compensatory proliferation that promotes chemical hepatocarcinogenesis. Cell 121, 977-990.
Meng, T. C., Fukada, T., and Tonks, N. K. (2002). Reversible oxidation and inactivation of protein tyrosine phosphatases in vivo. Mol Cell 9, 387-399.
Nakagawa, H., Maeda, S., Yoshida, H., Tateishi, R., Masuzaki, R., Ohki, T., Hayakawa, Y., Kinoshita, H., Yamakado, M., Kato, N., et al. (2009). Serum IL-6 levels and the risk for hepatocarcinogenesis in chronic hepatitis C patients; An analysis based on gender differences. Int J Cancer 125, 2264-2269.
Naugler, W. E., Sakurai, T., Kim, S., Maeda, S., Kim, K., Elsharkawy, A. M., and Karin, M. (2007). Gender disparity in liver cancer due to sex differences in MyD88-dependent IL-6 production. Science 317, 121-124.
Nowell, P. C. (1976). The clonal evolution of tumor cell populations. Science 194, 23-28.
Ogata, H., Kobayashi, T., Chinen, T., Takaki, H., Sanada, T., Minoda, Y., Koga, K., Takaesu, G., Maehara, Y., Iida, M., and Yoshimura, A. (2006). Deletion of the SOCS3 gene in liver parenchymal cells promotes hepatitis-induced hepatocarcinogenesis. Gastroenterology 131, 179-193.
Parekh, S., and Anania, F. A. (2007). Abnormal lipid and glucose metabolism in obesity: implications for nonalcoholic fatty liver disease. Gastroenterology 132, 2191-2207.
Parkin, D. M., Bray, F. I., and Devesa, S. S. (2001). Cancer burden in the year 2000. The global picture. Eur J Cancer 37 Suppl 8, S4-66.
Pham, C. G., Bubici, C., Zazzeroni, F., Papa, S., Jones, J., Alvarez, K., Jayawardena, S., De Smaele, E., Cong, R., Beaumont, C., et al. (2004). Ferritin heavy chain upregulation by NF-kappaB inhibits TNFalpha-induced apoptosis by suppressing reactive oxygen species. Cell 119, 529-542.
Pikarsky, E., Porat, R. M., Stein, I., Abramovitch, R., Amit, S., Kasem, S., Gutkovich-Pyest, E., Urieli-Shoval, S., Galun, E., and Ben-Neriah, Y. (2004). NF-kappaB functions as a tumour promoter in inflammation-associated cancer. Nature 431, 461-466.
Pitot, H. C. (1990). Altered hepatic foci: their role in murine hepatocarcinogenesis. Annu Rev Pharmacol Toxicol 30, 465-500.
Rebouissou, S., Amessou, M., Couchy, G., Poussin, K., Imbeaud, S., Pilati, C., Izard, T., Balabaud, C., Bioulac-Sage, P., and Zucman-Rossi, J. (2009). Frequent in-frame somatic deletions activate gp130 in inflammatory hepatocellular tumours. Nature 457, 200-204.
Roulston, A., Marcellus, R. C., and Branton, P. E. (1999). Viruses and apoptosis. Annu Rev Microbiol 53, 577-628.
Sakurai, T., He, G., Matsuzawa, A., Yu, G. Y., Maeda, S., Hardiman, G., and Karin, M. (2008). Hepatocyte necrosis induced by oxidative stress and IL-1 alpha release mediate carcinogen-induced compensatory proliferation and liver tumorigenesis. Cancer Cell 14, 156-165.
Sakurai, T., Maeda, S., Chang, L., and Karin, M. (2006). Loss of hepatic NF-kappa B activity enhances chemical hepatocarcinogenesis through sustained c-Jun N-terminal kinase 1 activation. Proc Natl Acad Sci USA 103, 10544-10551.
Salmeen, A., Andersen, J. N., Myers, M. P., Meng, T. C., Hinks, J. A., Tonks, N. K., and Barford, D. (2003). Redox regulation of protein tyrosine phosphatase 1B involves a sulphenyl-amide intermediate. Nature 423, 769-773.
Seki, S., Sakaguchi, H., Kitada, T., Tamori, A., Takeda, T., Kawada, N., Habu, D., Nakatani, K., Nishiguchi, S., and Shiomi, S. (2000). Outcomes of dysplastic nodules in human cirrhotic liver: a clinicopathological study. Clin Cancer Res 6, 3469-3473.
Sell, S., and Leffert, H. L. (2008). Liver cancer stem cells. J Clin Oncol 26, 2800-2805.
Siddiquee, K., Zhang, S., Guida, W. C., Blaskovich, M. A., Greedy, B., Lawrence, H. R., Yip, M. L., Jove, R., McLaughlin, M. M., Lawrence, N. J., et al. (2007). Selective chemical probe inhibitor of Stat3, identified through structure-based virtual screening, induces antitumor activity. Proc Natl Acad Sci USA 104, 7391-7396.
Takayama, T., Makuuchi, M., Hirohashi, S., Sakamoto, M., Okazaki, N., Takayasu, K., Kosuge, T., Motoo, Y., Yamazaki, S., and Hasegawa, H. (1990). Malignant transformation of adenomatous hyperplasia to hepatocellular carcinoma. Lancet 336, 1150-1153.
Tilg, H., Wilmer, A., Vogel, W., Herold, M., Nolchen, B., Judmaier, G., and Huber, C. (1992). Serum levels of cytokines in chronic liver diseases. Gastroenterology 103, 264-274.
Tong, M. J., el-Farra, N. S., Reikes, A. R., and Co, R. L. (1995). Clinical outcomes after transfusion-associated hepatitis C. N Engl J Med 332, 1463-1466.
Trikha, M., Corringham, R., Klein, B., and Rossi, J. F. (2003). Targeted anti-interleukin-6 monoclonal antibody therapy for cancer: a review of the rationale and clinical evidence. Clin Cancer Res 9, 4653-4665.
Valentino, L., and Pierre, J. (2006). JAK/STAT signal transduction: regulators and implication in hematological malignancies. Biochem Pharmacol 71, 713-721.
Wang, T., and Weinman, S. A. (2006). Causes and consequences of mitochondrial reactive oxygen species generation in hepatitis C. J Gastroenterol Hepatol 21 Suppl 3, S34-37.
Wanless, I. R. (1995). Terminology of nodular hepatocellular lesions. International Working Party. Hepatology 22, 983-993.
Weglarz, T. C., Degen, J. L., and Sandgren, E. P. (2000). Hepatocyte transplantation into diseased mouse liver. Kinetics of parenchymal repopulation and identification of the proliferative capacity of tetraploid and octaploid hepatocytes. Am J Pathol 157, 1963-1974.
Wen, D., Nong, Y., Morgan, J. G., Gangurde, P., Bielecki, A., Dasilva, J., Keaveney, M., Cheng, H., Fraser, C., Schopf, L., et al. (2006). A selective small molecule IkappaB Kinase beta inhibitor blocks nuclear factor kappaB-mediated inflammatory responses in human fibroblast-like synoviocytes, chondrocytes, and mast cells. J Pharmacol Exp Ther 317, 989-1001.
Wong, V. W., Yu, J., Cheng, A. S., Wong, G. L., Chan, H. Y., Chu, E. S., Ng, E. K., Chan, F. K., Sung, J. J., and Chan, H. L. (2009). High serum interleukin-6 level predicts future hepatocellular carcinoma development in patients with chronic hepatitis B. Int J Cancer 124, 2766-2770.
Zenewicz, L. A., Yancopoulos, G. D., Valenzuela, D. M., Murphy, A. J., Karow, M., and Flavell, R. A. (2007). Interleukin-22 but not interleukin-17 provides protection to hepatocytes during acute liver inflammation. Immunity 27, 647-659.
Zhang, J. Y., Green, C. L., Tao, S., and Khavari, P. A. (2004). NF-kappaB RelA opposes epidermal proliferation driven by TNFR1 and JNK. Genes Dev 18, 17-22.
Zhang, X., Zhang, G., Zhang, H., Karin, M., Bai, H., and Cai, D. (2008). Hypothalamic IKKbeta/NF-kappaB and ER stress link overnutrition to energy imbalance and obesity. Cell 135, 61-73.

Each and every publication and patent mentioned in the above specification is herein incorporated by reference in its entirety for all purposes. Various modifications and variations of the described methods and system of the invention will be apparent to those skilled in the art without departing from the scope and spirit of the invention. Although the invention has been described in connection with specific embodiments, the invention as claimed should not be unduly limited to such specific embodiments. Indeed, various modifications of the described modes for carrying out the invention which are obvious to those skilled in the art and in fields related thereto are intended to be within the scope of the following claims.

1-2. (canceled)

3. A method for producing an isolated population of mammalian hepatocellular carcinoma initiating cells (HICs), comprising
a) treating liver tissue from a mammalian subject with collagenase to produce a composition comprising a population of aggregated hepatocellular cells and a population of non-aggregated hepatocellular cells, and
b) isolating said population of aggregated hepatocellular cells from said composition, thereby producing an isolated population of mammalian hepatocellular carcinoma initiating cells (HICs).

4. The method of claim 3, wherein said mammalian subject is a mouse.

5. The method of claim 4, wherein said mouse is selected from the group consisting of a DEN-treated mouse, a mouse that lacks expression of TAK1, and a mouse that lacks expression of TAK1 and p38.

6. The method of claim 3, wherein said mammalian subject is human.

7. An isolated population of mammalian hepatocellular carcinoma initiating cells (HICs) produced by the method of claim 3.

8-12. (canceled)

13. A method for detecting and reducing the presence of hepatocellular carcinoma initiating cells (HICs) in a mammalian subject, comprising
a) detecting in a sample obtained from said subject one or more HIC marker gene that is identified by a method comprising determining the level of expression of a gene in
i) an isolated population of mammalian hepatocellular carcinoma initiating cells (HICs), and
ii) control non-cancerous cells,
wherein an altered level of gene expression in said HICs compared to said control cells identifies said gene as a HIC marker gene, and diagnoses said mammalian subject as comprising said HICs, and
b) administering to the diagnosed subject one or more agents that reduce one or more of the number of said HICs, and malignancy of said HICs.

14. The method of claim 13, wherein said detecting comprises determining an altered level of expression of said HIC marker gene in said sample compared to the level of expression of said HIC marker gene in a control sample.

15. The method of claim 14, wherein said control sample is selected from hepatic oval cell sample and hepatic normal cell sample.

16. The method of claim 13, wherein said HIC marker gene encodes an HIC cell surface marker antigen, and wherein said detecting comprises determining an altered level of expression of said HIC cell surface marker antigen in said sample compared to the level of expression of said HIC cell surface marker antigen in a control sample.

17. The method of claim 13, wherein said control sample is selected from hepatic oval cell sample and hepatic normal cell sample.

18. The method of claim 13, wherein said sample comprises liver tissue.

19. A method for identifying a test agent as reducing hepatocellular carcinoma initiating cells (HICs), comprising
a) contacting
i) the isolated population of mammalian hepatocellular carcinoma initiating cells (HICs) of claim 3, with
ii) said test agent, and
b) detecting at least one of
i) reduced number of said HICs, and
ii) reduced malignancy of said HICs,
wherein said detecting identifies said test agent as reducing hepatocellular carcinoma initiating cells (HICs).

20. The method of claim 19, wherein said test agent is selected from the group consisting of anti-cancer cytotoxin, antibody that specifically binds to a HIC cell surface marker antigen, RNA interference sequence that specifically binds to mRNA that encodes a HIC marker protein, and antisense sequence that encodes a HIC marker protein.

21. The method of claim 20, wherein said anti-cancer cytotoxin comprises a nucleotide sequence encoding herpes simplex virus thymidine kinase (HSVtk).

22. The method of claim 20, wherein said antibody that specifically binds to a HIC cell surface marker antigen is selected from the group consisting of antibody that specifically binds to CD44, and antibody that specifically binds to CD44v6.

23. The method of claim 20, wherein said test agent is covalently linked to an antibody that specifically binds to a HIC cell surface marker antigen.

24. The method of claim 20, wherein said test agent further comprises a liposome.

25. The method of claim 24, wherein said liposome further comprises an antibody that specifically binds to a HIC cell surface marker antigen.

26. The method of claim 25, wherein said HIC cell surface marker antigen is encoded by the HIC marker gene that is identified by a method for identifying a HIC marker gene, comprising determining the level of expression of a gene in
a) an isolated population of mammalian hepatocellular carcinoma initiating cells (HICs), and
b) control non-cancerous cells,
wherein an altered level of gene expression in said HICs compared to said control cells identifies said gene as a HIC marker gene.

27. A method for reducing hepatocellular carcinoma initiating cells (HICs) in a mammalian subject comprising administering to a subject in need thereof a therapeutic amount of an agent that reduces hepatocellular carcinoma initiating cells (HICs).

28. The method of claim 27, further comprising detecting at least one of
a) reduced number of said HICs, and
b) reduced malignancy of said HICs.

29. The method of claim 27, further comprising detecting reduced hepatocellular carcinoma (HCC) in said subject.

30. The method of claim 27, wherein said agent is identified by the method of claim 19.

31-37. (canceled)

38. The method of claim 13, wherein said one or more agents is selected from the group consisting of anti-cancer cytotoxin, antibody that specifically binds to a HIC cell surface marker antigen, RNA interference sequence that specifically binds to mRNA that encodes a HIC marker protein, and antisense sequence that encodes a HIC marker protein.
